(19) United States
(12) Patent Application Publication
(10) Pub. No.: US 2014/0217017 A1
(43) Pub. Date: Aug. 7, 2014

(54) Transforming energy and transportation into primary engines for reversing global warming and eliminating ocean acidification

(71) Applicants: Robert C. Fry, Omaha, NE (US); Sambhudas Chaudhuri, Manhattan, KS (US); Madeline J. Arnold, St. Joseph, MO (US); Barry M. Wroobel, Moorpark, CA (US); Grant H. Gower, Newburry Park, CA (US)

(72) Inventors: Robert C. Fry, Omaha, NE (US); Sambhudas Chaudhuri, Manhattan, KS (US); Madeline J. Arnold, St. Joseph, MO (US); Barry M. Wroobel, Moorpark, CA (US); Grant H. Gower, Newburry Park, CA (US)

(73) Assignee: CLIMATE RESTORATION TECHNOLOGIES, INC., Omaha, NE (US)

(21) Appl. No.: 13/999,195

(22) Filed: Jan. 27, 2014

Related U.S. Application Data
(60) Provisional application No. 61/962,955, filed on Nov. 20, 2013.
(60) Provisional application No. 61/960,954, filed on Oct. 1, 2013.
(60) Provisional application No. 61/760,224, filed on Feb. 4, 2013.

(51) Int. Cl. C02F 3/32; C12M 1/00

(52) U.S. Cl. CPC C02F 3/322; C12M 21/02
USPC 210/611; 435/292.1

(57) ABSTRACT

The invention encompasses multi-stage naturally amplified global-scale carbon dioxide capture systems combining basic capture from (CC—carbon capture) clean-coal-fired and CC gas-fired power plants, natural-gas reformation systems, cement plants, outdoor air, home and building flues, incinerators, crematoriums, blast-furnaces, kilns, refineries, factories, oil gasification systems and coal gasification systems which yield concentrated carbon dioxide, with a collective, globally distributed capture capacity of up to 3 GtC/yr, feeding the captured carbon dioxide into land-based invention stage-1 bioreactors for rapid, selective, high capacity conversion to a high-density, fast-sinking marine algae by means of accelerated photosynthesis and/or coccolithogenesis (calcification) consuming carbon dioxide as the algae bloom, and transporting the stage-1 bioreactor-produced algae to seaports for seeding the oceans at regular intervals in stage-2 operations-at-sea to produce naturally amplified 14 GtC/yr algal blooms at sea, the stage-2 operations circumventing classic prior-art (and natural) ocean fertilization limits of low bloom rate, grazers eating algae seed before it blooms, interfering buoyant strains which don't clear the photic zone to allow light penetration for multiple blooms per year, and proximal post-bloom anoxia. A total invention CO₂ capture and safe storage capacity of 17 GtC/yr (land and sea) is projected during fair-weather, and a 40% foul weather down-time allowance ensures that an average 10 GtC/yr of impact capture would result. If emissions are concurrently capped by at 12 GtC/yr by 2023, with invention-assisted reduction to 6 GtC/yr by 2050, 3 GtC/yr by 2062, and 1 GtC/yr by 2078, atmospheric CO₂ will be reduced to 280 ppm by 2075.

A spin-off technology includes hydrogen (H₂) production by natural-gas reformation—enough H₂ to fuel a significant fraction of transportation by 2050. Spin-off side benefits of the invention system include restoring ideal ocean pH and re-proliferating decimated marine populations, restoring them to levels last seen in the 18^{th} to mid-19^{th} centuries. Additional spin-off applications of invention bioreactor algal production include silage, animal feed, feed supplements, fertilizer, biofuels, food for fish and mollusk farming, cleansing lakes and rivers of bacteria and agricultural run-off, and elimination of coastal water HAB's (harmful algae blooms), such as the notorious “red tide” in Florida.

This application claims benefit of provisional applications, Nos. 61/962,955 filed on Nov. 20, 2013; 61/960,954 filed on Oct. 1, 2013; and 61/760,224 filed on Feb. 4, 2013.

### FIELD OF THE INVENTION

This multi-stage invention system relates to the science of global climate change and ocean acidification, the related field of geo-engineering, and more specifically to global climate restoration, ocean revitalization, and fueling transportation with hydrogen (H₂). The invention further relates to capture and storage of carbon dioxide (CO₂) from power-plants, natural-gas-reformation systems, oil gasification systems, coal gasification systems, cement plants, refineries, factories, blast furnaces, kilns, outdoor air, home and building flues, incinerators, crematoriums, and other significant anthropogenic sources of CO₂ emission. The invention further relates to high efficiency conversion of captured CO₂ to algae in land-based bioreactors and to global-scale, naturally amplified CO₂ capture by ocean algal blooming from bioreactor seed. Reduction in atmospheric CO₂ will reverse global warming, restore climate, and automatically restore ideal ocean pH. Increased ocean algal blooming will feed the marine food chain and help restore decimated marine populations.

In addition to the invention bioreactors contributing to climate restoration and ocean revitalization, other applications will include high capacity algal production for silage, animal feed, feed supplements, fertilizer, biofuels, food for fish and seafood farming involving species of fish or mollusk which directly feed on algae, and bottom-rung food for fish farming involving predator fish (as seafood) such as compano and cobia which feed on lower marine life (e.g, brine shrimp). In the latter case, invention algal production will feed brine shrimp or other lower chain marine species in separate tanks, raising them for secondary feeding to predator fish.

Invention fresh-water algal production can further aid in revitalization of inland lakes and rivers by removal of nitrogen and phosphorus compounds added by agricultural runoff. Clearing major rivers of agricultural runoff will stop coastal water harmful algae blooms (HAB's) such as the notorious “red tide” in Florida which is fed by agricultural runoff at major river delta outflows.

### BACKGROUND OF THE INVENTION

Climate destabilization, ocean acidification, and the related acceleration of an impending marine die-off are anticipated to become the biggest challenges of the 21^{st} century. All three problems are related to rising atmospheric concentrations of the greenhouse gas (GHG) carbon dioxide (CO₂) produced by fossil-fuel burning, cement production, and agriculture. With 90-95% statistical certainty, multiple national and international agencies have reported that global warming in the 21^{st} century is real, undeniable, anthropogenic (man-made), and getting steadily worse (with acceleration) with each passing decade since the 1970's. 2000-2010 was the warmest decade on record.

Atmospheric carbon dioxide (CO₂) accounts for ˜56% of today's warming. CO₂ is still rising and accelerating toward near-term 450 parts-per-million (ppm) tipping points [Hansen, et. al., 2008, 2009; and Cao and Caldeira, 2008] for setting irreversible catastrophic global warming in motion, plus related acceleration of an impending marine die-off. Atmospheric CO₂ reached 400 ppm in May 2013[Keeling, et. al., 2013] which is the highest level in 13 million years [Solomon, et. al., 2007]. We calculate that, barring intervention, CO₂ will reach the 450 ppm twin tipping points by 2028.

CO₂ emissions from fossil fuel combustion and cement production rose to 9.7 billion tons per year (carbon measure, GtC/yr) in 2012 [Rapier, 2012, and McGee, 2013]. Emissions are accelerating at approximately 3.5% annually [Solomon, et. al., 2007; Allison, et. al., 2009; Rapier, 2012; and McGee, 2013]. In an unchecked scenario, we estimate emissions would reach 17 GtC/yr by 2034.

Significant intervention is required. The solution should target a 21^{st} century return to 280 ppm CO₂ and ideal ocean pH. It should avoiding impending short-term 450 ppm tipping points and ideally restore 280 ppm atmospheric CO₂ by 2075.

Our modeling calculations show that, in order to succeed, humanity must cap CO₂ emissions at 12 billion tons carbon per year (12 GtC/yr) by 2023, and then cut emissions to 1 GtC/yr by 2078 as illustrated by FIG. 1, curve 1. We must also develop 17 GtC/yr of CO₂ capture capacity (curve 2), which allows a 40% margin for foul weather, interruptions, delays, etc., and apply the remaining 10 GtC/yr of actual impact capture (curve 3) from 2027-2072, plus an optional 10-year time-contingency extension allowance (4) for further unexpected delays and interruptions.

Our calculations suggest that a FIG. 1 combination of emissions caps, cuts, and CO₂ capturing would avoid tipping points (5)—see PPM CO₂ accumulation impact curve 6-by about 25 ppm (7), with a maximum (8) of 425 ppm CO₂ occurring in 2023. Subsequent annual reductions would eventually restore 280 ppm (9) atmospheric CO₂ by 2075. They would also restore ideal ocean pH by lowering dissolved carbonic acid, which depends on atmospheric CO₂. Meeting the new CO₂ emission, capture, and atmospheric accumulation-reduction targets of FIG. 1 (curves 1, 2, 3, and 6) will be a monumental task, and an increasingly evident reality if we are to avoid impending tipping points, reverse global warming, and revitalize oceans.

It should be noted that FIG. 1 capture targets and impacts significantly exceed the scale and capacity of single-stage, unamplified, prior-art CO₂ remediation technologies, several of which were reviewed in The Economist, Mar. 17, 2012, pp. 74, 75. Meeting FIG. 1 targets will require a new generation of significantly amplified multi-stage CO₂ capture technologies. That is where future global climate stabilization and energy development resources should be focused.

As of this writing, there remains a need for amplified global CO₂ capture capacity exceeding our projected 2023 emissions cap of 12 GtC/yr, with a “fair weather” contingency capture and safe storage capacity preferably attaining 17 GtC/yr. Several prior-art air-capture systems which frequent the news media (The Economist, op. cit.) would have estimated capture costs of $330-$800/ton of CO₂, without considering storage. With a FIG. 1 total accumulated capture requirement exceeding 1.7 tera-tons actual CO₂ by 2075, this would amount to 560-1,360 tera-dollars (560 trillions-1,360 trillions of dollars) for capture alone—clearly more money than is available. The Economist (op. cit.) estimates prior-art CCS capture systems to potentially be able reduce those costs by 10× (e.g., to the range of 56-136 trillion dollars, which might be somewhat more affordable if cost-shared by 60 countries and amortized over 50 years), but they have neither the required CO₂ capture capacity nor the required safe storage capacity.

This summarizes the overall impracticality of prior-art single-stage geo-engineering systems and their non-viability for meeting FIG. 1 targets. In reality, the prior-art single-stage systems won't be scalable for preventing a rise in atmospheric CO₂ to the 450 ppm twin tipping points for catastrophic warming or ocean acidification. They won't even likely be able to significantly delay the anticipated crossing date (2028) for exceeding those tipping points. CO₂ warming is much bigger (and more urgent) than is generally acknowledged (or understood), and no adequate, affordable solution has yet been offered. Prior-art geo-engineering systems are single-stage, don't exhibit capture amplification, and can't deliver anywhere near the required 17 GtC/yr or 1.7 tera-ton overall CO₂ capture and safe storage capacity. Even if prior-art systems had the capacity, their costs would exceed humanity's ability and willingness to pay.

Nature can provide the only realistic (and affordable) possibility for vast CO₂ capture amplification and safe storage. However, nature's immense capacities for dealing with excess atmospheric CO₂ have yet to be harnessed. Nature's capture and storage mechanisms are currently working, but not nearly at full capacity. In order to avoid the 450 ppm twin tipping points (5) for catastrophic CO₂ warming, to restore the pre-industrial atmosphere of 280 ppm (9), and to restore ideal ocean pH, a means of triggering nature's immense capture and storage mechanisms to operate at full capacity must be quickly found. Nature's full CO₂ capture and safe storage capacities, evident in past ice-ages, would hypothetically be adequate for solving our 21^{st} century warming problem, but they're essentially dormant now and, barring intervention, we'll cross the 450 ppm tipping points (5) by 2028-long before the next ice-age.

Humanity's task is to awaken (and safely accelerate) nature's full CO₂ capture and safe storage capacities, ramping up from 2020-2027 (˜30,000 years ahead of the next scheduled ice age) and ending in 2072, per curve 3 of FIG. 1. That sounds impossible, but the invention systems described herein place that goal within reach.

Since the 1980's, at least 14 others (prior-art) have shared our vision of awakening nature's sleeping “green giant”, to answer humanity's dire need in the present warming crisis [Boyd, 2007; Mankin, 1995; Abraham, et. al., 1999; EisenEx, 2000; Tsuda, et al., 2001, 2004; Barber, et. al., 2002, 2007; Johnson, 2002, 2004; Walter, et. al., 2004; Castellani and Gardiner, 2005; Mehrtens, 2009; Bhattachatya, 2009; and Pielke, Jr, 2012], but (so far) this prior-art has been unsuccessful. The green giant still sleeps. Our task is to contrive an early (interglacial) anthropogenic trigger to awaken the nature's green giant beginning in 2020 and ramping up through 2027, without waiting for the next ice age, in order for nature to remove ˜145 ppm CO₂ (from the anticipated 2023-capped level of −425 ppm (see FIG. 1, curve 6)), eventually restoring the atmosphere to the pre-industrial level of 280 ppm CO₂ (9) without actually triggering an ice-age. Fourteen prior-art attempts [Boyd, 2007; Mankin, 1995; Abraham, et. al., 1999; EisenEx, 2000; Tsuda, et. al., 2001, 2004; Barber, et. al., 2002, 2007; Johnson, 2002, 2004; Walter, et. al., 2004; Castellani and Gardiner, 2005; Mehrtens, 2009; Bhattacharya, 2009; and Pielke, Jr, 2012] to accomplish that were well-meaning, but they all failed for a variety of reasons which this invention can circumvent.

In the long term, rock-weathering has sufficient capture capacity, but it's far too slow to solve our immediate, urgent CO₂ warming problem and it cannot realistically be accelerated to offset annual anthropogenic CO₂ emissions of 9.7-12 GtC/yr in modern times. We'd cross the 450 ppm CO₂ tipping point (5) for runaway warming by 2028, long before natural rock-weathering could make a significant impact. The prior-art, crushed-rock CO₂ mineralization proposal of Lackner, et. al. (Brookings Papers, 2005, 2, 215-81, and Annual Review of Energy and the Environment (2002), 27, 193-232) is a related concept which would increase the surface-area-per-ton with crushed rock exposure, but it is deemed likely to fall far short of the required capacity for storing 10 GtC/yr CO₂ (impact) to be captured each year from 2027-2072 (FIG. 1, curve 3). A FIG. 1, curve 3 total storage requirement for more than 450 billion tons of excess atmospheric carbon (1.65 tera-tons actual CO₂ excess) is needed in reducing atmospheric CO₂ to 280 ppm (9). This capture and storage requirement remains unfulfilled, and no viable prior-art proposal has yet been identified with prospects for achieving it.

With rock-weathering being too slow, oceans are the only realistic remaining option for leveraging vast amplification in CO₂ capture and safe storage. Ocean capture of CO₂ occurs in two ways. One way is CO₂ solubility in the ocean as carbonic acid (H₂CO₃). In oceans, most of the carbonic acid produced by CO₂ dissolution dissociates to form bicarbonate ion (HCO₃), but that process liberates hydrogen ion (H^{f}) which ultimately lowers the pH of the oceans (e.g. pH 8.33→pH 8.1) and produces damaging ocean acidification (e.g., pH 8.17—a CO₂ related problem) at today's excessive atmospheric CO₂ level. The ocean's capacity for safe capture of atmospheric CO₂ by its solubility (as a mixture of H₂CO₃ and HCO₃⁻ (mostly HCO₃⁻)) has already been exhausted in modern times. The oceans have already exceeded their maximum tolerable acidity (low pH (˜8.17)), killing 80% of global coral and threatening to soften (partially dissolve) the carbonate exoskeletons of a variety of marine life (tipping point=450 ppm atmospheric CO₂), so another means of leveraged CO₂ capture by the oceans must be found.

One prior-art proposal (www.c-questrate.com) is to “lime-the-sea”, thereby raising its pH and increasing the capacity for ocean solubility of CO₂. However, this would initially release massive amounts of extra CO₂ because this lime would be first produced by heating vast quantities of limestone mined from the Australian Nullarbor plain (releasing its long-naturally-sequestered CO₂), before the resulting lime could be distributed at sea. Although c-questrate.com authors claim an offsetting second phase (recapturing more CO₂ at sea than was originally released), there would be a significant hazard (mortal danger to marine life) of excessive localized alkalinity during lime dispersal and mixing at sea, which c-questrate.com didn't adequately address, and we also view the initial release of massive amounts of extra CO₂ as being too risky.

The second means of ocean capture of CO₂ is large-scale photosynthesis (algal blooming). This is nature's true green giant. Prodigious ocean algal blooming occurred repeatedly during multiple ice-ages in the last 800,000 years, and atmospheric CO₂ was drawn down by large scale photosynthesis at sea, with atmospheric CO₂ accumulations dropping as low as 190 ppm. Under ice-age conditions, the oceans were cold and no thermo-cline existed. This allowed continuous upwelling of nutrients originating from lava oozing from active ocean-floor rifts (in deeper water) to replenish top-water micro-nutrients which got used up in support of prodigious ice-age algal blooming in the uppermost photic zone.

However, in our modern interglacial (warm) climate, not much algae actually blooms over the course of a year in the vast majority of global ocean area. This is indicated by the existence today of vast ocean deserts where satellite images (FIG. 2) show nearly no algae currently blooming over the course of each year in the open seas south of Seattle, Spain, and Japan. This occurs because warm seas are stratified. They exhibit a thermo-cline which inhibits upwelling of replacement micro-nutrients as they become depleted in the upper photic zone by the first algal bloom of spring. Once the photic zone is depleted of micro-nutrients following an initial bloom, they aren't rapidly replaced because nutrient upwelling from colder, deeper water is blocked by the thermo-cline. This is the reason why very little algal blooming occurs over much of the open oceans over the course of a year. FIG. 2 shows that nature's green giant is currently sleeping.

To create large, repeating open-sea algal blooms (i.e., awaken the green giant) in today's warm climate, nutrient depletion must be overcome. One prior-art proposal suggests actively pumping nutrient-rich colder water from below the thermo-cline. Our calculations indicate this wouldn't be practical for feeding (required) 14 GtC/yr ocean algal blooms. Blocked by a prevailing ocean thermo-cline in warm stratified seas, large-scale replenishment of micro-nutrients in the photic zone will have to be achieved by some means other than upwelling (or pumping) from deeper water. Recognition of this led to 14 prior-art attempts at ocean fertilization (adding micro-nutrients) since the 1980's. Although laboratory tests were initially promising, other factors prevented success at sea, and none of the prior-art attempts yielded sustainable ocean blooming or significant sustained CO₂ capture [Boyd, 2007; Mankin, 1995; Abraham, et. al., 1999; EisenEx, 2000; Tsuda, et. al., 2001, 2004; Barber, et. al., 2002, 2007; Johnson, 2002, 2004; Walter, et. al., 2004; Castellani and Gardiner, 2005; Mehrtens, 2009; Bhattachatya, 2009; and Pielke, Jr, 2012]. A need yet remains to circumvent remaining factors which prevent ocean fertilization from succeeding.

Ocean algal blooming has the potential to become a powerful force for atmospheric CO₂ removal. It has a repeating paleo-climatic history of pulling atmospheric CO₂ down as low as 190 ppm by accelerated photosynthesis during multiple ice-ages (800,000 BC-8,000 BC). However the accelerated algal draw-down mechanism of the ice-ages required cold, de-stratified seas (with no ocean thermo-cline) which allowed continuous upwelling of replenishment nutrient from deeper water as algal blooming consumed nutrients in the photic zone (the top 10-100 meters where light penetration drives photosynthesis). Even then, once a natural (cold-sea) algal draw-down (of CO₂) cycle began (triggered by initial cooling at a Milankovitch solar orbital cycle minimum), it typically took 40,000 years to draw atmospheric CO₂ from 250 ppm down to 190 ppm as each ice-age developed [Solomon, et. al., 2007]. Nobody wants another ice age, but these paleo-climatic indications at least demonstrate that ocean algal blooming is capable of pulling enough CO₂ (e.g., 145 ppm) out of the atmosphere to reverse global warming if sufficient replenishment nutrient were available as photic zone blooming progresses.

It has therefore been abundantly demonstrated in natural history, that ocean algal blooming is capable of eventually accomplishing our goal, and this is where we must look for the necessary CO₂ capture capacity. The oceans are really the only thing big, powerful (i.e. nature's green giant), and responsive enough to achieve the required CO₂ capture and safe storage capacity. Everything else (e.g., all prior-art man-made systems) will be too small and ineffectual in the face of 12 GtC/yr global emissions (projected by 2023). The oceans have done it many times before [Solomon, et. al., 2007], albeit more slowly than our current need requires, and it is humanity's task to determine how they may be stimulated to do it again, with considerable acceleration this time, before the atmosphere reaches 450 ppm CO₂. Our difficult problem is that we are currently in a warm period where ocean capture of CO₂ by accelerated algal blooming lies essentially dormant (see FIG. 2), owing to warm stratified seas with nutrient upwelling blocked by thermocline, and not enough time remains to naturally accelerate ocean blooming before the 450 ppm tipping point is reached. A need therefore remains for significant anthropogenic intervention—essentially an invention trigger and significant acceleration means involving ocean capture of CO₂.

To create large, repeating open sea algal blooms in today's warm climate, a vast FIG. 2 micro-nutrient depletion would have to be overcome. The micro-nutrient would have to—be replenished by a means other than upwelling from deep water. This accounts for 14 prior-art human intervention attempts at anthropogenic (man-made) ocean fertilization since the 1980's [Boyd, 2007; Mankin, 1995; Abraham, et. al., 1999; EisenEx, 2000; Tsuda, etat, 2001, 2004; Barber, et. al., 2002, 2007; Johnson, 2002, 2004; Walter, et. al., 2004; Castellani and Gardiner, 2005; Mehrtens, 2009; Bhattacharya, 2009; and Pielke, Jr, 2012]. Ocean fertilization appeared promising in a number of the small scale prior-art laboratory tests, but other factors prevented their success at sea, and none of the prior-art attempts yielded large ocean blooms or globally scalable CO₂ capture.

The first of the “other factors” (besides photic-zone nutrient depletion in warm, stratified seas) preventing anthropogenic stimulation of nature's full CO₂ draw-down capacity via prodigious algal blooming rates capable of rapidly delivering ice-age magnitude draw-down by 2075 (i.e., a 145 ppm reduction in CO₂) in today's warm climate is low blooming rate with natural algae seed levels occurring at an average of only 0.1 mg/m³ (chlorophyll measure) for the year, as illustrated by FIG. 2 satellite imagery. Even under ideal conditions, the upward-bending non-linear algal growth curve can't yield rapid blooming rates from such a low starting point. Even if sufficient nutrient were available, starting from only 0.1 mg/m³ (chlorophyll-a measure) of natural algal seed wouldn't produce blooming sufficient to reach a 14 GtC/yr ocean capture target for CO₂ by the end of each year. Prior-art ocean fertilization attempts all dosed nutrient-only into the seas, and the 0.1 mg/m³ natural seed levels weren't sufficient to support high bloom rates, regardless of nutrient dosing. There remains a need for a means of providing higher initial seed levels, seeding much higher on the non-linear growth curve to boost blooming rates and CO₂ capture capacity toward 14 GtC/yr.

The second of other factors (besides low natural seed levels and photic-zone nutrient depletion in warm stratified seas) preventing anthropogenic stimulation of nature's full CO₂ draw-down capacity via prodigious ocean algal blooming rates capable of delivering ice-age magnitude draw-down in today's warm climate is buoyancy of natural algae species (e.g, blue-green algae), creating a persistent floating light-block following initial blooming. Once an initial bloom develops, buoyancy can prevent it from sinking to clear the photic zone in time for subsequent blooms to develop and raise the global CO₂ capture rate to 14 GtC/yr before the end of each year. Instead, subsequent photic zone algal blooming would get stalled by persistent optical opacity after a single initial bloom of natural buoyant strains, and the multiplicity of subsequent blooms required to raise total annual blooming to 14 GtC/yr cannot develop. There remains a need for suppressing blooming of buoyant strains of algae in the ocean and means of selectively inducing high-density, heavier-than-water, fast-sinking strains of algae to bloom preferentially, so rapid photic zone clearing (after each bloom) can enable development of 12 blooms/yr to boost accumulated bloom rates and CO₂ capture to 14 GtC/yr.

The third of other factors (besides buoyancy, low natural seed levels, and photic-zone nutrient depletion in warm stratified seas) preventing anthropogenic stimulation of nature's full CO₂ draw-down capacity via prodigious ocean algal blooming rates capable of delivering ice-age magnitude draw-down in today's warm climate is the remaining need to provide higher initial seed levels, much higher on the non-linear growth curve to boost ocean blooming and CO₂ capture toward 14 GtC/yr, which gives rise to a yet-to-be-fulfilled need for selectively producing large quantities of high-density, heavier-than-water, fast-sinking marine algae seed on land, and then dispersing it at sea. This creates a remaining further need for increasing the output capacity of bio-reactors (on land) to produce high-density ocean algae seed. A need further remains for algal bio-reactors that will continuously produce prodigious quantities of the ocean algae seed (on land) at a bio-reactor harvest output port in a free-flowing (non-agglomerated, non-colonized) concentrated liquid suspension.

The fourth of other factors (besides limited seed production capacity of land-based bioreactors, low natural ocean seed levels, buoyancy, and photic-zone nutrient depletion in warm stratified seas) preventing anthropogenic stimulation of nature's full CO₂ draw-down capacity via prodigious ocean algal blooming rates capable of rapidly delivering ice-age magnitude draw-down in today's warm climate is the current overbalanced (and starving) ocean populations of Antarctic krill and zooplankton grazers such as copepods which hide below the photic zone during the day (out-of-range (hidden) from visual predators), but then come charging up from the deep each night to devour any algae they can find in the (night-dark) “photic” zone. For example, the 2009 prior-art attempt by the PolarStern research vessel (Albert Wegner Institute, Germany) to fertilize a large algae bloom with iron in Antarctic seas was thwarted by copepods devouring the entire haptophyte starter bloom overnight before it had a chance to bloom further and capture significant CO₂. Copepods and other zooplankton are voracious feeders on algae. With an overbalanced population, their appetites for algae are currently estimated at 2 GtC/yr. They can essentially devour all of the available algae starter seed essentially overnight, before it has a chance to bloom, leaving no prospect for development of amplified blooms leading to 14 GtC/yr CO₂ capture. Their predators (baleen whales and adult fish (e.g. menhaden, pilchard, herring, shad, anchovies, etc.)) have been hunted or over-fished to a point where only 10-30% of former predator populations remain. With 70-90% of the predators gone owing to excessive whaling and commercial over-fishing, grazer populations have grown out of control and this makes it difficult to avoid seed algae getting eaten by grazers before it has a chance to bloom anywhere near a 14 GtC/yr target. There remains a need for means to prevent overpopulated, starving Antarctic krill, copepods, and other zooplankton grazers from eating all of the available seed algae in a single night (following dispersal), before it has a chance to bloom and capture up to 14 GtC/yr of CO₂ by the end of each year.

The fifth of other factors (besides zooplankton grazers, limited land-based bioreactor seed production capacity, low natural seed levels, buoyancy, and photic-zone nutrient depletion in warm stratified seas) likely to prevent anthropogenic stimulation of nature's full CO₂ draw-down capacity via prodigious ocean algal blooming rates capable of rapidly delivering ice-age magnitude draw-down in today's warm climate is proximal post-bloom anoxia, which can occur following the death of large ocean algal blooms. Decay, following death of a large algal bloom, can trigger secondary microbial (bacterial) blooming which consumes dissolved oxygen, creating an oxygen depletion zone that can kill marine life in the vicinity. Oxygen depletion can extend all the way down to the shallow ocean floor in coastal waters. This is not so much a preventer of accelerated algal blooming (per se), but it is environmentally unsound and it would likely raise popular and regulatory agency objections which would likely activate (or lead to) legal and/or legislative intervention to block allowance of further ocean seeding or fertilization which would be required for large-scale ocean blooming to achieve the 14 GtC/yr ocean blooming and CO₂ capture target. There remains a need for means to prevent proximal post-bloom anoxia following the death of large ocean algal blooms, so that dissolved oxygen levels remain high, and legal and/or legislative blocking become unnecessary, and accelerated ocean blooming may be allowed to proceed toward a 14 GtC/yr CO₂ capture target.

It should be noted that all 5 of the above other factors (besides photic-zone nutrient depletion in warm stratified seas) preventing anthropogenic stimulation of nature's full CO₂ draw-down capacity via prodigious ocean algal blooming rates capable of rapidly delivering ice-age magnitude draw-down in today's warm climate must be circumvented in order for accelerated ocean blooming to proceed toward a 14 GtC/yr CO₂ capture target. There remains a need for the circumvention of photic zone nutrient depletion and the all 5 other factors.

Global CO₂ emissions are currently 9.7 GtC/yr and our FIG. 1 (curve 1) trend analysis projects they will rise to 12 GtC/yr by 2023. In order for 14-17 GtC/yr CO₂ contingency capture (curve 2) to successfully reduce atmospheric CO₂ accumulation to 280 ppm (9) by 2075 (curve 6), global carbon emissions (curve 1) must also be capped at 12 GtC/yr by 2023, and then gradually reduced to 6 GtC/yr by 2050, 3 GtC/yr by 2062, and 1 GtC/yr by 2078. This is illustrated by FIG. 1, in which the required emissions cap and reduction schedule is graphed as curve 1 (for fossil fuel consumption and cement production emissions), the required net annual capture impact is 10 GtC/yr (curve 3), and the recommended fair-weather 17 GtC/yr total (land and sea) contingency carbon capture schedule is graphed as curve 2, and the resulting atmospheric CO₂ accumulation impact is graphed as curve 6. The atmospheric CO₂ accumulation curve (6) is computed from the annual difference between the recommended CO₂ impact capture curve (3) and the required CO₂ fossil and cement emissions curve (1), with the annual differential expressed in ppm and subtracted (with sign) from the atmospheric accumulation existing in each previous year, plus added annual correction for natural sinks and land-use change emissions [LeQuere, et. al., 20133]. FIG. 1 is a graphical illustration of the desired goal of reversing CO₂ warming by 2075. No prior-art systems or system combinations have demonstrated FIG. 1 capacities, CO₂ removal performance, or future potential for achieving the FIG. 1 capacities and performance. In order to reduce atmospheric CO₂ to 280 ppm (9) on the illustrated FIG. 1 schedule (by 2075) and avoid the 450 ppm tipping points (5) for runaway warming, there remains a need for CO₂ capture and safe storage capacity equaling impact capture curve 3 of FIG. 1 (and its illustrated schedule). There also remains a need for means of capping and reducing global CO₂ emissions equaling curve 1 of FIG. 1 (and its illustrated schedule).

Regarding means of capping and reducing global CO₂ emissions according to curve 1 of FIG. 1, the largest single challenge (largest source of anthropogenic CO₂ emissions) is coal-fired power plants. Prior-art new-generation, pilot-stage, coal-fired electric power plants currently capture ˜50% of their CO₂ emissions as super-critical fluid CO₂ (SCF-CO₂). The pilot-plan for SCF-CO₂ is to pump it into underground porous rock structures for storage, or pump it to the bottom of the deep sea, or use it as a shale-fracking agent. This prior-art pilot-stage CO₂ removal and storage technology is commonly referred to as carbon capture and sequestration (CCS), and the overall combination of power-plant and CC (carbon-capture) pilot-stage systems is commonly called “clean-coal”. Although CC power-plants (clean-coal) would exhibit a smaller carbon footprint than older non-CC coal-fired power plants, a substantial positive carbon footprint still remains for prior-art pilot-stage clean-coal and CC power-plants. In addition, satisfactory porous rock structures for underground SCF-CO₂ storage are scarce and difficult to find. Storage integrity is not guaranteed. Storage integrity could be breached in a seismic event or upheaval, and stored SCF-CO₂ could rapidly decompress and suddenly release to atmosphere as concentrated CO₂ (locally lethal) and creating an abrupt return of the warming impact from greenhouse gases (GHG) thought to have been previously removed. A similar containment breach may be envisioned for shale-fracking use of SCF-CO₂. Significant environmental concerns and objections would also arise from pumping SCF-CO₂ directly to the ocean floor.

There remains a need for substantial carbon footprint reduction (or elimination) for clean-coal and CC coal-fired power plants. There also remains a need for carbon footprint reduction (or elimination) for gas-fired power plants and for combination gas-and-coal-fired power plants. In order to avoid the 450 ppm twin tipping points for runaway warming and catastrophic ocean acidification, and also to reduce atmospheric CO₂ accumulation to 280 ppm by 2075 (FIG. 1, curve 6), there also remains a need for amplified CO₂ capture from CC power plants in which multi-stage globally amplified capture removes up to 8 times more CO₂ than the power plants produce. There also remains a need for safer, more readily available carbon storage which is not subject to seismic release or causing environmental damage.

Regarding the means of capping and reducing global CO₂ emissions equaling curve 1 of FIG. 1, fossil fuel burning in transportation is the second largest source of CO₂. There remains a need for alternative transportation fuels which do not emit CO₂ as vehicles operate. Hydrogen (H₂) is one such fuel, but it is typically produced by prior-art natural-gas reformation, in which natural gas (mostly methane (CH₄)) is initially injected into high temperature steam. Steam (H₂O) cracks off the carbon (from CH₄) as carbon monoxide (CO), leaving 3 hydrogen (H₂) molecules to be separated and compressed for transportation fuel. In a second reformation process step, the CO byproduct remaining after hydrogen separation is further reacted in a second step with low temperature steam in a water-gas-shift reaction that produces CO₂ and another hydrogen molecule. That hydrogen is also separated and compressed for transportation fuel.

Similar processes could also be used to make hydrogen transportation fuel from oil or coal. In this case, the process is referred to as gasification. In either oil gasification or coal gasification, the precursor fuels (oil or coal) would be converted to syngas, a mixture of hydrogen (H₂) and carbon monoxide (CO) via partial oxidation under partially oxygen-starved conditions. The hydrogen may be separated and compressed for transportation fuel. The CO byproduct would once again be reacted in a second step with low temperature steam in a water-gas-shift reaction that produces CO₂ and another batch of hydrogen. The 2^{nd} batch of hydrogen may again be separated and compressed for transportation fuel.

Although hydrogen-powered cars essentially do not (themselves) have a carbon footprint, all three prior-art hydrogen fuel production processes (natural-gas reformation, oil gasification, and coal gasification) have a substantial carbon footprint, owing to their final CO₂ process byproduct. In the case of natural gas reformation, this gives prior-art hydrogen fueling of transportation an overall carbon footprint which is only ˜20-30% improved over vehicles which burn gasoline, which negates about 70-80% of the climate-and-ocean-restoring benefit of hydrogen-powered cars.

In fact, considering typical 1.5% leakage losses in the overall drilling, fracking, distribution, storage, and usage of natural-gas (CH₄) in the prior-art methane reformation process for making hydrogen, and further considering that leaked (raw, unburned) CH₄ exhibits 25-72× greater GHG warming potency (per molecule) than CO₂, . . . prior-art hydrogen fueling of transportation wouldn't actually exhibit a lower climate warming footprint than gasoline, once overall CH₄ leakage is taken into account.

There remains a need for substantial carbon footprint (CO₂) reduction or elimination for the natural-gas (CH₄) reformation process for making hydrogen (H₂). A similar need remains for carbon footprint reduction while making hydrogen by oil gasification and/or coal gasification.

In order to avoid the 450 ppm twin tipping points for runaway warming ocean acidification, to meet the targets of FIG. 1 and reduce atmospheric CO₂ accumulation to 280 ppm by 2075 (FIG. 1, curve 6), there remains a need for amplified CO₂ capture from natural-gas reformation production of hydrogen, and from oil gasification and coal gasification production of hydrogen, in which multi-stage globally amplified capture would remove as much as 15 times more CO₂ than the natural-gas reformation, oil gasification, and coal gasification processes (for hydrogen production) produce as their byproduct, thereby significantly offsetting accumulated CH₄ leakage and vaulting hydrogen to a front-runner position in alternative transportation fuel development.

Regarding additional means of capping and reducing global CO₂ emissions (curve 1, FIG. 1), cement production is a significant source. There remains a need for substantial carbon footprint reduction for cement production. To help avoid the 450 ppm twin tipping points for runaway warming and ocean acidification, and to help reduce atmospheric CO₂ accumulation to 280 ppm by 2075, there remains a need for amplified CO₂ capture from cement plants in which multi-stage globally amplified capture removes 8-15 times more CO₂ than the cement production process emits.

Regarding additional means of capping and reducing global CO₂ emissions (Curve 1, FIG. 1), home and building flues, blast furnaces, kilns, refineries, factories, incinerators, and crematoriums are significant sources. There remains a need for substantial carbon footprint reduction for these CO₂ sources. In order to avoid the 450 ppm twin tipping points for runaway warming and ocean acidification, and to reduce atmospheric CO₂ accumulation to 280 ppm by 2075, there remains a need for amplified CO₂ capture from home and building flues, blast furnaces, kilns, refineries, factories, incinerators, and crematoriums in which multi-stage amplified capture removes 8-15 times more CO₂ than these sources emit.

There also remains a need for amplified CO₂ capture from outdoor air (over land), in which multi-stage globally amplified capture removes 15 times more CO₂ (globally) than single-stage air-capture initially removes.

There remains a need for the CO₂ captured from the power-plants, the natural-gas reformation process for hydrogen production, the oil gasification process for hydrogen production, the coal gasification process for hydrogen production, cement plants, blast furnaces, kilns, refineries, factories, home and building flues, incinerators, crematoriums, and outdoor air (over land), to be coupled separately or in combination with CO₂ captured from any or all of these systems into high capacity algal bioreactors. The particular remaining need is for globally distributed arrays of the coupled bioreactors to exhibit sufficient CO₂ conversion capacity to continuously produce non-buoyant (high density, heavier-than-water, fast-sinking) marine algae at a collective rate of 1-3 GtC/yr. A final remaining need exists for the 1-3 GtC/yr algal bioreactor high-density marine seed algae harvest output to be widely dispersed (with micro-nutrients) over approximately 70% of the oceans to seed them high on the ocean growth (blooming rate) curve, stimulating, accelerating, and selectively amplifying vast ocean algal blooms which capture up to 14 GtC/yr atmospheric CO₂ at sea, with combined land-and-sea fair-weather CO₂ capture rates of 17 GtC/yr (10 GtC/yr impact), and in which buoyant algal species do not significantly interfere or effectively compete, and in which krill and zooplankton grazers (including copepods) are prevented from eating enough of the seed to prevent it from blooming to 14 GtC/yr at sea, and in which proximal post-bloom anoxia is suppressed, such that an overall compound multi-stage amplification factor of 15× is achieved for CO₂ capture and the 17 GtC/yr CO₂ capture curve (2) of FIG. 1 (10 GtC/yr impact curve 3) are all safely achieved, thereby enabling the capped and reduced CO₂ accumulation curve (6) of FIG. 1 to be achieved (in the event that the prerequisite capped and reduced emission curve (1) of FIG. 1 is also achieved) with atmospheric CO₂ being reduced to 280 ppm by 2075 (9) as indicated by FIG. 1, adding up to a capture and safe storage (by the ocean) of a total of 0.45 tera-ton (carbon measure) or 1.65 tera-tons actual CO₂ by 2075.

In related areas, there remains a need for ocean revitalization in terms of ideal pH restoration (elimination of ocean acidification) and recovery of decimated marine populations. There remains a further need for high capacity algal production to supply silage, animal feed, feed supplements, fertilizer, biofuels, food for fish and seafood farming involving species of fish or mollusk which directly feed on algae, and bottom-rung food for fish farming involving predator fish (as seafood) such as compano and cobia which feed on lower marine life (e.g, brine shrimp). In the latter case, there remains a need for high capacity algal production to feed the brine shrimp in separate tanks, raising the shrimp for secondary feeding to predator fish.

Finally, there remains a need for revitalization of inland lakes and rivers by removal of nitrogen and phosphorus compounds added by agricultural runoff. The remaining need for clearing major rivers of agricultural runoff is related to a need to eliminate coastal water harmful algae blooms (HAB's), such as the notorious “red tide” in Florida, which are fed by agricultural runoff at major river delta outflows.

Nuclear energy is an ideal long-term solution [Hansen, 2009, Stone, 2013], but its global expansion is currently experiencing delay and significant public opinion backlash, notably in the USA, Germany, and Japan. Correction of widespread public misperception and dispelling unwarranted fears will take time, and sufficient global nuclear expansion, though vitally important in the long term [Hansen, 2009, Stone, 2013], would come too late to forestall impending 450 ppm CO₂ tipping points—which, barring significant intervention, may arrive as early as 2028. Interim innovation is needed from another corner.

### SUMMARY OF THE INVENTION

This invention system offers the above mentioned required innovations and global intervention means, including the required CO₂ capture amplification and capacity. Our basic invention concept and interim vision (for the period 2020-2072) is to make each ton of CO₂ captured on land from carbon-based energy, transportation, and industry drive additional capture of up to 14 more tons of atmospheric CO₂ at sea. A total of 10 GtC/yr (impact) invention-system-induced, ocean-amplified CO₂ capture capacity are projected, along with 17 GtC/yr fair-weather contingency capture capacity and an accumulated total capture and natural safe storage capacity of 0.45 tera-tons carbon (1.65 tera-tons as CO₂) over the 45 year period from 2027-2072 (with initial ramp-up from 2020-2027). That is the required capture period at 10 GtC/yr (impact) illustrated by FIG. 1 target requirements, and globally scaled and distributed units of the invention-system (globally deployed) are collectively anticipated to have the required capacity—delivered by invention-system-induced 15× ocean amplification. A 40% contingency for foul weather down-time, delays and interruptions is provided for via a 17 GtC/yr invention system fair-weather capture capacity. Natural storage will occur primarily as sea-floor carbonates, which is completely natural, safe, stable, and essentially permanent. There will also be a substantial living carbon storage pool as currently decimated marine populations are restored in the invention-induced process of ocean revitalization. As atmospheric CO₂ is reduced, global warming will recede and ocean acidity will simultaneously diminish.

### 1. Power-Plant CO₂ Conversion to Algae

As an initial example embodiment of the invention system, FIG. 3 illustrates a coal-fired or gas-fired CC (carbon capture) power plant (10) which captures at least 50% of its CO₂ as supercritical fluid carbon dioxide (SCF-CO₂) (11). That much is prior art—essentially pre-existing pilot-stage technology.

(Note: numbers in parentheses in the following pages refer to numerically labeled items in the associated drawings.)

However, instead of the conventional CC pilot-plan for burying SCF-CO₂ in subterranean porous rock structures, pumping it to the sea floor, or using it as a shale-fracking aid, our FIG. 3 invention system embodiment begins by scrapping the prior-art burial pipe (12) and diverting SCF-CO₂ output to a high-pressure surface reservoir (13). From there, SCF-CO₂ is piped (14) or transported to invention decompression chambers (16) where it is decompressed in two stages (15-17) into invention photo-bioreactors (18) which are algae conversion silos that efficiently convert CO₂ to high-density marine algae. Only one invention conversion silo is illustrated, but manifold (19) can subdivide and disperse SCF-CO₂ to a large array of identical invention silos.

Internal algae silo design is discussed later. The design and required number of invention silos would be sufficient to convert injected CO₂ to high-density (heavier-than-water) marine algae as fast as SCF-CO₂ is produced by the CC power plant.

This algae silo (18) is a high speed, high efficiency invention photo-bioreactor with marine algae being continuously produced and removed to the harvest output (20) as fast as it blooms. Marine algae from the harvest output are to be transported to sea-ports and widely dispersed, along with metered nutrient doses, across the oceans as seed to stimulate and accelerate invention-system-induced secondary ocean blooming on a much larger scale.

Each ton of SCF-CO₂ produced by the CC power plant is to be invention-converted to marine algae (20) for seeding massively amplified invention-system-induced secondary ocean blooming with species-selective bloom dominance and capture of 14 more tons of atmospheric CO₂ at sea. The invention system illustrated here is a prelude to invention-induced 1400% ocean-amplified CO₂ capture. At 50% initial CC capture efficiency, a 700% negative carbon footprint would be imparted to CC power production by ocean amplification triggered by the invention system. CC power plants would thereby be transformed into primary engines for atmospheric CO₂ removal and their operations and fuel suppliers would become key enablers for the reversal of global warming and the elimination of ocean acidification.

### 2. Future Transportation CO₂ Conversion to Algae

FIG. 4 illustrates another invention system embodiment, which is conversion of CO₂ byproduct (40) from hydrogen-producing (37) natural-gas (30) steam crackers (33, 34) to marine algae (20). If upstream-enabled by the algal seed conversion of FIG. 4 and by the invention-system-induced ocean-amplified secondary capture of massive amounts of atmospheric CO₂ at sea, hydrogen (H₂) (37) could become a primary ground transportation (38) fuel of the future. A hydrogen-powered automobile (38) is depicted, but the invention system is equally applicable to hydrogen-powered vans, buses, trucks, trains, ships, or planes.

Hydrogen (H₂) does not release CO₂ on consumption. However, CO₂ is liberated during hydrogen fuel production (37) by a two-step natural-gas reformation process (30, 33-37):

Natural Gas Reformation
CH₄+H₂O₍ₛₜₑₐₘ₎→3H₂+CO (T1=700-1100° C.; (30, 33))
CO+H₂O₍ₛₜₑₐₘ₎→H₂+CO₂ (T2=130° C. (34))

Unfortunately, CO₂ byproduct from the second process reaction (34) conventionally negates about 70-80% of the potential climate-and-ocean restoring benefit of hydrogen (37) as a transportation fuel. CO₂ byproduct emission (34) is a current limitation of prior-art hydrogen (H₂) production (37) by natural gas reformation (30, 33-37).

The FIG. 4 invention system embodiment separates (39, 40) steam-cracker byproduct CO₂ as SCF-CO₂. The rest of FIG. 4 is the same as FIG. 3—in which SCF-CO₂ is collected (13) and decompressed (14-17) into algae photo-bioreactor silos (18) for rapid conversion to high-density marine algae (20), which is to be seeded into the ocean for triggering invention-induced amplified secondary blooming and massive secondary CO₂ capture at sea. For each ton of CO₂ captured from the steam-cracker of FIG. 4, the invention system would enable another 14 tons of atmospheric CO₂ to be captured at sea. This would impart a 1400% negative carbon footprint to future hydrogen production and to hydrogen transportation in general. Future transportation could thereby be transformed into another primary engine for atmospheric CO₂ removal, global warming reversal, and the elimination of ocean acidification. An invention-system-induced large negative carbon footprint could vault hydrogen (37) to a front-runner position among alternative fuels development while leveraging increased precursor natural-gas (30) production as a key enabler for global climate restoration and ocean revitalization.

3. Hydrogen from Coal Gasification or Oil

Future hydrogen for transportation could also be invention-system-produced via coal-gasification or from oil. In these cases, partial oxidation of the coal or oil to form syngas, a mixture of carbon monoxide and hydrogen, under partially-oxygen-starved conditions would be the first step. Partial oxidation of the coal or oil would replace the first (T₁) steam cracking process reaction (30-33) of natural gas reformation, as in the following simplified reactions (which have been intentionally abbreviated to omit sulfur and minor-element constituents of the precursor fuels).

Partial Oxidation→Syngas
Coal: C₂₄H₁₂+12O₂→6H₂+24CO
Oil: C₁₂H₂₄+6O₂→12H₂+12CO

The water-gas-shift reaction (steam reaction, at T₂=130° C.; (34)) would still apply to convert syngas CO to CO₂ with production of additional hydrogen.

Water-Gas Shift Reaction
CO +H₂O₍ₛₜₑₐₘ₎→H₂+CO₂ (130° C.; (34))

Following each of the above process reactions, CO and/or CO₂ would be separated (35, 39) and hydrogen (H₂) compressed (36) for transportation fuel (37), the same as in FIG. 4. Remaining CO₂ would be invention-system captured as SCF-CO₂, with the rest of FIG. 4 being the same as before, for final CO₂ conversion (18) to marine algae (20) to be used in invention system seeding of secondary ocean blooming in order to effect massively amplified capture of additional atmospheric CO₂ at sea. As before, this would impart a 1400% negative carbon footprint to future hydrogen production (37) and to hydrogen transportation (38) in general, and further contribute to vaulting hydrogen to a front-runner position in alternative fuels development while leveraging increased precursor natural-gas, oil, and coal production as key enablers for global climate restoration and ocean revitalization.

### 4. Stage-1 Land Capture and Seed Conversion Summary

FIG. 5 is an overview of invention system land-capture technologies for industry and other CO₂ sources, including transportation, and energy, and which collectively comprise stage-1 of our invention system atmospheric CO₂ reduction concept and the overall reversal of global warming and ocean acidification. Beginning at the upper right (50), we have already discussed CC clean-coal and gas-fired power plants, as well as natural-gas (CH₄) steam-crackers (54) and oil or coal-gasification (syngas) reactors (54) which could produce hydrogen (H₂) (37) for future ultra-clean transportation (38). Other land-based CO₂ sources (57) such as cement production, industrial sources, refineries, factories, home and building heating, blast furnaces, kilns, crematoriums, incinerators, etc. could be similarly subjected to CO₂ capture. All of these systems (50, 52, 54, 57) could converge their byproduct SCF-CO₂ (51, 53, 58-64) into arrays of algae silos (65) which would collectively enable conversion of the CO₂ to marine algae seed (20) at the global rate of up to 3 GtC per year. Overall, these algal-terminus processes could upstream-enable a significant increase in natural-gas markets for CC power production and in gas, oil, and coal markets for supplying precursor feedstock to hydrogen-fuel production for future zero-emissions transportation (38).

### 5. Ocean-Amplified CO₂ Capture

FIGS. 3-5 illustrated stage-1 of our climate restoration plan involving arrays of land-based algae silos converting captured CO₂ from concentrated sources to fast-sinking marine algae for seeding the oceans to stimulate and accelerate much larger secondary ocean algal blooming in stage-2 (71—FIG. 6) with 1400% amplified atmospheric CO₂ capture (77) at sea (76). To begin stage-2 (71), FIG. 6 indicates that the stage-1 (70) land-harvested algae (20, 72) would be loaded into stasis-supporting cargo containers and transported to sea-ports (73). The containers would be transferred to cargo ships for distribution to floating seed repositories (74) located across the open seas. The oil and gas industry already has the knowledge and resources to design and implement habitable floating seed repositories. Seed-boats (75) would fan-out from the repositories (74) to widely disperse seed, along with metered nutrient doses, across 70% of Earth's oceans (76) on a monthly repeating basis.

Because algae+nutrient will be seeded instead of nutrient alone, and because the amount of available seed from stage-1 land-harvest (65, 20, 70, 72) would be large, ocean seeding (71, 75, 76) could begin significantly higher on its nonlinear growth curve than was possible in previous prior-art attempts at ocean fertilization [Boyd, 2007; Mankin, 1995; Abraham, et. al., 1999; EisenEx, 2000; Tsuda, et al., 2001, 2004; Barber, et. al., 2002, 2007; Johnson, 2002, 2004; Walter, et. al., 2004; Castellani and Gardiner, 2005; Mehrtens, 2009; Bhattacharya, 2009; and Pielke, Jr, 2012]. This would stimulate substantially accelerated secondary ocean blooming (76-79) with species-selective dominance of the blooms by high-density, fast-sinking marine algae such as siliceous diatoms and/or Emiliania huxleyi, a calcareous exoskeletal coccolithophore which is heavier-than-water. Bloom dominance may be further enhanced by nutrient selection.

Secondary ocean blooms approaching light penetration (algal opacity) limits, and visible from outer space, are anticipated within 8-14 days of initial seeding. At that point, metered nutrient doses would be calculated to run out. Heavier-than-water algae would then die and rapidly sink to clear the photic zone in preparation for next month's re-seeding. Species-selective bloom dominance and rapid sinking would prevent formation of a persistent floating algal light-penetration block. Species-selective bloom dominance and heavy exoskeletal armor (coccolith plates) may further enable (dead) E. huxleyi to quickly sink below the ocean thermocline to the deep sea floor where low temperatures (near 0° C.) and armored plating can effectively slow and/or suppress secondary bacterial blooming. Dead algae could be preserved on the cold deep-sea floor until they become buried under a steady accumulation of ocean sediments—often referred to as marine “snow”. Low temperature algal preservation and burial could prevent post-bloom anoxia from developing in the open seas. This hypothesis is crucial for validating anthropogenically-induced large-scale ocean blooming of E. huxleyi or siliceous diatoms. It requires proof-of-concept testing for verification, and that will certainly be worthwhile to explore.

Undecayed, rapidly sinking heavy algae may also get eaten as they descend and benthic creatures may feed on these algae as they reach the deep-sea floor. In the absence of eutrophication and post-bloom anoxia, general marine life may be expected to thrive as a result of bottom-rung feeding of the deep-water ocean food chain with large amounts of nutritious, freshly bloomed, well preserved, naturally refrigerated algae on a monthly repeating basis. Our hypothesis is that this may possibly lead to a generalized ocean revitalization, extending well beyond the benefits of climate restoration and eliminating ocean acidification.

In any case, 15-fold amplified photosynthesis and coccolithogenesis are anticipated with massive, species-selective, secondary algal blooming at the rate of 14 GtC/yr at sea—divided into 12 blooms per year. Multiple blooms would be enabled by a combination of accelerated blooming and accelerated post-bloom sinking with rapid clearance of the photic zone prior to each monthly reseeding, owing to high seed levels and species-selective ocean bloom dominance by heavier-than-water species of algae (siliceous diatoms and/or E. huxleyi). A correspondingly large amount of atmospheric CO₂ would be captured at sea (76-79) during ocean-amplified photosynthesis and coccolithogenesis. A total land-and-sea CO₂ capture (70, 71) capacity of 17 GtC/year is anticipated globally. If global CO₂ emissions are also controlled as described earlier, 17 GtC/year ocean-amplified capture capacity would be sufficient to avoid the near-term 450 ppm tipping points and reverse global warming→eliminating ocean acidification and restoring 280 ppm CO₂ by 2075. The crucial element is two-stage 1400% amplified ocean capture of CO₂.

Anticipated results of ocean amplified capture of CO₂ are illustrated in the FIG. 7 graphs. The Y-axis represents either the amount of algae to be seeded into the ocean annually or the anticipated total amount of CO₂ that will be captured. The dashed curve represents our intended ocean seeding level at 1 GtC/yr (82) with an added early seeding “bump” (80) which would briefly be 3 GtC/yr. The seed “bump” is needed to offset anticipated voracious zooplankton grazer feeding which could otherwise lead to our seed being devoured by currently overbalanced populations of copepods, krill, etc. before it has a chance to bloom and capture CO₂. Global grazer appetites for algae are currently estimated at 2 GtC/yr. Setting the front end seed “bump” (80) at 3 GtC/yr should exceed grazer appetites, thereby enabling excess algae to bloom prodigiously and capture 14 GtC/yr of CO₂ in the process. Front-end availability of 3 GtC/yr of seed from stage-1 land-based invention system algae silos is therefore a critical enabler of ocean-amplified CO₂ capture. Without that extra seeding level, currently overpopulated grazers could devour smaller amounts of seed before they have a chance to bloom.

With general marine life thriving as a result of suppressing post-bloom anoxia and feeding the bottom rung of the ocean food chain with large amounts of algae on a monthly repeating basis, the return of marine predators may be anticipated, accompanied by a re-balancing of grazer and predator populations. As the predators eat the grazers back into normal population balance, the front end 3 GtC/yr seed-bump (82) may no longer be necessary and it should be possible to subsequently reduce seeding to 1 GtC/yr as indicated by section 82 of the dashed seed curve (FIG. 7). The extra 2 GtC/yr of land-harvested algae could then be reallocated to profitable land-use (organic fertilizer, silage, animal feed, fish farm feed, agricultural runoff control, etc.)

In response to dashed curve ocean seeding (80, 82), nature is expected to respond by doing the “heavy lifting” illustrated by the solid curve (81) which indicates massively amplified ocean capture of atmospheric CO₂. This is what will be required to meet the CO₂ targets of FIG. 1. The amplified capture curve (81) of FIG. 7 essentially matches the targeted 17 GtC/yr contingency capture curve (2) in FIG. 1, except that curve 2 showed an added (optional) 10-year time-contingency extension allowance for unexpected delays. Such a time extension would be accomplished by extending the 1 GtC/yr dashed seed curve (82) of FIG. 7 by another 10 years, if that becomes necessary.

The anticipated constant land-and-sea capture capacity of 17 GtC/yr (81) is nominally recommended for maintenance from 2027-2072 as shown in FIG. 7. 3 GtC/yr of this capacity would be stage-1 land capture and 14 GtC/yr would be stage-2 amplified ocean capture. This curve includes a built-in 40% margin for bad weather delays and other interruptions—equivalent to the 40% capture offset seen between curves 2 and 3 and the vertical double-headed arrow of FIG. 1. It is important to note that global scaleup to meet the targets of FIG. 1 requires a FIG. 7 level of CO₂ capture amplification.

### 4. Preliminary Summary Conclusions

Based on the modeling calculations graphed in FIG. 1, a formula for success would include developing a 17 GtC/yr global CO₂ contingency capture capacity (curve 2) with 10 GtC/yr impact (curve 3), and applying that from 2027-2072, while concurrently capping CO₂ emissions initially at 12 GtC/yr by 2023 and cutting/stabilizing emissions to 1 GtC/yr by 2078 (curve 1). These actions would collectively result in atmospheric CO₂ being capped at <425 ppm by 2023 and then gradually reduced to 280 ppm by 2075 (curve 6). The tipping points will have been (narrowly) averted (7) and the stage set for global warming reversal (following a thermal lag delay). As secondary benefits, ocean acidification would be automatically eliminated and teeming populations of marine life, last seen in the 18^{th} and 19^{th} centuries, could be restored. All of this would occur with traditional energy providers and this invention system technology as key enablers for global warming reversal and ocean revitalization.

The solution to global warming and ocean acidification begins with offering a path that allows green advocates and energy producers to begin working together. New partners in success would include green advocates as constructive solutions educators, climate scientists, geo-engineers, algae specialists, marine biologists, oceanographers, oil, gas, and coal industries as primary scaleup implementers, and this invention system technology. Key ingredients are green advocates' passion, oil, gas and coal industry influence and resources, this invention system technology, hydrogen-powered transportation, and the long-term global proliferation of nuclear energy.

In a non-limiting example, the invention system encompasses multi-stage naturally amplified global-scale carbon dioxide capture systems combining initial land-based man-made capture systems (either prior art or invention systems) which yield concentrated carbon dioxide at their output, feeding that output into land-based man-made (invention) bioreactors for rapid, selective conversion to at least one high density, fast-sinking, heavier-than-water species of marine algae by means of accelerated photosynthesis and/or coccolithogenesis (calcification) consuming carbon dioxide while the algae bloom as in FIGS. 3-5 and FIGS. 8-13, and in which, referring to FIGS. 6 and 7, the land-based (invention stage-1) bioreactor-produced algae is transported in invention stasis-supporting cargo containers (73) to seaports to enable seeding the oceans (75, 76, 80, 82) at regular intervals in invention stage-2 operations-at-sea (71) to produce much larger (naturally 15× amplified) algal blooms at sea (76-79, 81), and in which the stage-2 invention operations (71) dispense (stage-1-produced (70)) seed-algae (20, 72)+micronutrient into the ocean 75, 76, 80, 82) instead of just micronutrient-alone, and in which the stage-2 (71) selectively amplified ocean blooms (81) are essentially uniformly composed of the same algae species as the seed produced on land (65, 20, 72) in stage-1 (70 and FIG. 5), and in which proximal post-bloom anoxia following bloom cycles of the stage-2 amplified algal blooms at sea (71, 81) is optionally prevented by aerator boats which pass through post-bloom regions while bubbling compressed air or oxygen through long, weighted hoses into the sea to depths of within 5 meters of the coastal sea floor, in a non-limiting example, in order to reaerate the coastal waters and prevent proximal post-bloom anoxia from secondary microbial blooming at the end of each stage-2 (71, 80-82) ocean algae bloom cycle. It is anticipated that invention system reaeration would only be required in coastal waters in zones where a continental shelf presents a relatively warm, shallow sea floor. It is not anticipated that deep water aeration in invention-system-seeded zones of the open seas where no continental shelf exists, the waters are much deeper, temperatures at the deep-sea floor are typically 4 degrees centigrade or less, and sedimentation rates (of marine “snow”) of approximately 1 mm/year would bury the sunken, dead, cold-preserved, invention-system-seeded-blooms of heavier-than-water algae before post-bloom anoxia has a chance to develop.

In preferred embodiments, the invention system would exhibit output capacity of a globally-proliferated multiplicity of the FIGS. 5, 6 invention stage-1 land-based bioreactors (65) sufficient to enable 1-3 GtC/yr of FIGS. 6, 7 stage-2 ocean seeding (71, 75, 80, 82) with the selected species of high density, fast-sinking stage-1 algae (70, 20, 72) to occur at elevated seed levels (75, 80, 82) substantially exceeding low levels of interfering buoyant algae species which naturally occur (FIG. 2) in the ocean, the invention-elevated seed levels (FIGS. 5-7) selectively accelerating the stage-2 (71) ocean blooming rate of the selected species of high density, fast-sinking stage-1 (70) bioreactor seed algae (only), essentially shortening the overall ocean bloom cycle and enabling the selected strain of high density, fast-sinking algae to essentially dominate the amplified stage-2 (71) ocean algal blooms, substantially overshadowing low-level interfering buoyant algae strains to a degree that they cannot compete effectively or contribute significantly to the invention stage-2 amplified ocean algae blooming and CO₂ capture (81). It is anticipated that invention-system nutrient selection, e.g., phosphate-free nutrients for E. huxleyi ocean seeding in a nonlimiting example, could further enhance invention-system species selective bloom dominance of ocean-amplified, invention-system-seeded, heavier-than-water blooms.

In preferred embodiments, multi-stage naturally amplified global-scale carbon dioxide capture invention systems would foster amplified stage-2 ocean algae blooms of sufficient weight density to enable rapid post-mortem sinking and photic zone clearing at the end of each accelerated bloom cycle, the cycle being limited in duration by invention-system-accelerated bloom rate and the amount of available micronutrient, and the rapid (post mortem) algae sinking and clearing of the photic zone enabling invention system reseeding of the photic zone within a foreshortened time period, the species-selective dominance of amplified stage-2 high density oceanic algae blooms and their rapid (post-mortem) sinking and clearing of the photic zone conspiring to avoid formation of persistent floating light blocks from interfering buoyant strains of algae which might otherwise occur to prevent effective invention-system reseeding the following month.

In preferred embodiments, multi-stage naturally amplified global-scale carbon dioxide invention capture systems with shortened bloom cycles would enable more frequent reseeding of stage-2 ocean algae blooms, creating a plurality of ocean algal blooms (e.g., 12 blooms/yr in a non-limiting example), the plurality further geometrically amplifying annual carbon dioxide capture by an additional (multiplicative) plurality factor equaling the number of ocean blooms achieved by the plurality each year in a non-limiting example, and exceeding carbon dioxide capture by interfering natural buoyant algae strains to a degree equaling the plurality factor. In a non-limiting example, an invention plurality factor of 12× would apply, raising carbon dioxide capture by invention-system-enhanced ocean blooming to 14 GtC/yr.

In one embodiment of Type #1 (SCF-CO₂ path) multi-stage naturally amplified global-scale carbon dioxide invention capture system, a super-critical fluid CO₂ starting point is envisioned as in FIG. 3. In a non-limiting preferred embodiment of the Type #1 invention system, the SCF-CO₂ starting point is a new-generation, prior-art gas-fired or clean coal-fired electric power generating plant (10) featuring capture of at least a fraction of its carbon dioxide emissions as a concentrated form of carbon dioxide (11) which may be stored (13) and/or invention-processed (14-17) for continuously infusing at least one invention system stage-1 bioreactor (18), or a multiplicity of invention bio-reactors (19, 18), with elevated levels of carbon dioxide that induce prodigious bioreactor blooming and output harvest (20), with subsequent invention stage-2 ocean amplification (FIGS. 6, 7) imparting an overall substantially negative carbon footprint to CC (carbon capture) gas-fired or CC coal-fired power plants (10), such that a multiplicity of tons of carbon dioxide are captured by invention system stage-2 (71, 80-81) at sea for each ton of carbon dioxide produced by the stage-1 power plant (10), the multiplicity being determined by the overall invention-system-compounded amplification factor (82, 81) of the multi-stage capture system (70, 71) using whole-earth carbon accounting. The FIGS. 3, 6 combination multi-stage invention-system embodiment will contribute significantly to the (FIG. 7) 17 GtC/yr CO₂ capture curve (81), which meets the (FIG. 1) 17 GtC/yr contingency capture target (2) and the 10 GtC/yr impact capture target (3) and the prior-art clean-coal or gas-fired CC power plant (10 (FIG. 3)) will thereby be enabled to contribute significantly to achieving the FIG. 1 emissions cap (1), which (in turn) will contribute to the reduction curve (1) via the at least 50% reduced stack emissions (FIG. 3, item 22) of CC gas-fired or CC coal-fired power plants (10-CC power plants with capture (11) of at least 50% of their CO₂ emissions) replacing conventional coal-fired and gas-fired power plants.

In a second preferred embodiment of Type #1 multi-stage naturally amplified global-scale carbon dioxide invention capture systems, the FIG. 3 generic SCF-CO₂ source (10, 11) is a cement plant (not separately illustrated) featuring capture of a fraction of its CO₂ emissions as a concentrated form of carbon dioxide (11) which may be stored (3) and/or processed (4-7) for continuously infusing the at least one invention stage-1 bioreactor (18) with elevated levels of carbon dioxide that induce prodigious bioreactor blooming and output harvest (20), with subsequent invention system stage-2 (71) ocean amplification (FIG. 6) imparting an overall substantially negative carbon footprint to cement plants, such that a multiplicity of tons of carbon dioxide are captured by stage-2 at sea (71, 81) for each ton of carbon dioxide produced by the stage-1 cement plant (10), the multiplicity being determined by the overall invention compounded amplification factor (81, 82) of the multi-stage capture system (70, 71) using whole-earth carbon accounting.

In a third preferred embodiment of the Type #1 multi-stage naturally amplified global scale carbon dioxide invention capture system, FIG. 4 illustrates that the SCF-CO₂ starting point (30-35, 39, 40) is a natural-gas reformation system for producing hydrogen (36, 37), in which natural-gas (essentially methane, CH₄) is injected (30) into steam (33, 34), and in which the carbon (in CH₄) is cracked off in two stages as carbon dioxide and the residual hydrogen (37) is molecular hydrogen (H₂) gas, and in which the 2^{nd} stage carbon dioxide may be invention-separated (39) from the hydrogen (36, 37) and concentrated (40) for continuously infusing (13-17) the at least one invention stage-1 bioreactor (18) with elevated levels of carbon dioxide that induce prodigious bioreactor blooming and output harvest (20), with subsequent invention stage-2 ocean amplification (FIGS. 6, 7) imparting an overall substantially negative carbon footprint to natural gas reformation and hydrogen production, such that a multiplicity of tons of carbon dioxide are captured by stage-2 (71) at sea for each ton of carbon dioxide produced by the stage-1 natural gas reformation system (FIG. 4) for producing hydrogen (37), the multiplicity being determined by the overall invention-compounded amplification factor (81, 82 (FIG. 7)) of the 2-stage capture system (FIG. 6) using whole-earth carbon accounting, and in which the residual hydrogen (36, 37) from stage-1 (FIG. 4) may be used as transportation fuel for hydrogen-powered vehicles (38) such as fuel-cell powered vehicles or vehicles with internal combustion engines operating on hydrogen. Invention-amplified CO₂ capture will contribute strongly to the FIG. 1 capture curves (2, 3). Because the invention system also enables global-scale proliferation of H₂ production for transportation fueling (FIG. 4 (37, 38)), there will be a separate substantial contribution to emissions reduction and its corresponding impact on curve 1 (FIG. 1).

In certain FIGS. 3-5 embodiments of the multi-stage naturally amplified global scale carbon dioxide invention capture system, carbon dioxide separation and concentration (11, 39, 40, 51, 53, 58, 59, 61) may be achieved by liquefaction. In other FIGS. 3, 4—Type #1 invention system embodiments, carbon dioxide separation and concentration (11, 39, 40, 51, 53, 58, 59, 61) may be achieved by super-critical fluid CO₂ capture technology (CC clean-coal, or CC gas-fired, in a non-limiting FIGS. 3, 6 CC power plant example, or a nonlimiting FIGS. 3, 6 CC cement plant, or CC methane reformation in a FIGS. 4, 6 non-limiting example). FIG. 5 CC blast furnace, kiln, refinery, factory, and other examples may all be envisioned (57) within the scope of the invention system.

In Type #2 embodiments of the multi-stage naturally amplified global scale carbon dioxide invention capture system, FIG. 9 illustrates that carbon dioxide separation and concentration may be achieved by invention reaction of CO₂-laden gas mixtures (120, 122) with sodium hydroxide (NaOH, caustic soda, lye (126, 128, 129)) in a thin film (129) reactor (121) which functions as a lye scrubber, so that the CO₂ is captured by the downward flowing lye film (129) as sodium bicarbonate solution (130) which is then drained (130) and the CO₂ re-released by subsequent invention closed-system (139) acidification (131-133) of the bicarbonate solution (130) and infusion (140, 142) of the re-released carbon dioxide (138) into invention stage-1 bioreactors (algae conversion silos (18, 90)) where it feeds algal blooming to produce the stage-1 seed (20) for stage 2 ocean-amplified blooming (FIGS. 6, 7). One preferred embodiment of the FIG. 9 Type #2 land-based algal conversion—lye capture path for CO₂ is illustrated in FIG. 10 which is a home or filling station embodiment of hydrogen production by methane reformation. This preferred embodiment captures CO₂ from the methane reformation process (149, 150, 122) in a thin film reactor (121) exposing the reformation gas mixture (122, 123) to a downward flowing lye film (129), capturing the spent reaction product bicarbonate solution (130), and storing it in a pickup vessel (151) for later transport (152) to a district receiving station (153) which feeds the same acidification (131-133) and closed-system CO₂ re-release chamber (139) and land-based algae conversion silo (18, 90) as before. This embodiment also couples its silo algae output (20) to stage-2 (FIG. 6) for 15× ocean amplification as before. By this means, the FIGS. 10, 6 multi-stage invention imparts home or filling station hydrogen fueling of transportation with a 1500% negative carbon footprint, using whole earth carbon accounting. As in the case of FIGS. 3-5, the FIGS. 9, 10 embodiments (with FIGS. 6, 7 ocean amplification) will contribute to the amplified CO₂ capture curves (2, 3, 81) of FIGS. 1, 7 but the FIG. 10 invention boost to globalization of hydrogen-powered transportation will also lower global emissions, which will contribute strongly to meeting the emissions reduction target curve (1), FIG. 1.

Other preferred embodiments of FIG. 9 land-based algal conversion type #2 (lye capture path) invention are illustrated in FIG. 11, which is a lye scrubber for home and building flues. It would also work for incinerators and crematoriums (not shown). It's based on exposing CO₂-laden flue gases (163, 164, 166, 167) in a rising vortex counter-flow (123) to a downward flowing lye film (129) produced by lye overflowing (128) a standpipe (127) within a thin film reactor (121). If needed, auxiliary cooling air (not shown) may optionally be mixed with the hot flue gases (163, 166) prior to entering the thin film reactor (164, 167). The lye film (129) flowing down the outside of the standpipe (127) absorbs CO₂ from the rising vortex counter-flow of flue gases (123), converting the CO₂ to sodium bicarbonate solution which then drains out of the reactor at 130. Stripped air (124) exits the thin film reactor at 168 and continues in the flue exhaust (170). If needed, flue gases may be pulled through the thin film reactor (121) with an exhaust fan (169) pulling on the stripped air (168) outlet. The bicarbonate collection vessel (151) of FIG. 11 may be considered a district pickup vessel like the pickup vessel (151) in FIG. 10 to be delivered to the district acidification system (153, 131-142) and algae conversion silos (18, 90) of FIG. 10, and the silo output (20) may be further amplified by stage-2 operations at sea (FIGS. 6, 7). This system will impart 15× ocean amplification to land-based CO₂ capture (FIG. 11) from home and building flues, incinerators, and crematoriums. The amplified CO₂ capture will contribute to capture curves 2, 3, 81 of FIGS. 1, 7. A flue-based emission reduction may also be credited which in turn will contribute strongly to meeting the targets of emission reduction curve 1 (FIG. 1).

One preferred embodiment of Type #2 land-based algal conversion (NaOH starter path) is illustrated in FIG. 12 which is an outdoor air embodiment of invention CO₂ capture. It features a large scale invention bin (180) which houses a lye fountain (184-188) through which large amounts of CO₂-laden outdoor air are drawn (182, 183). Air enters the lye fountain bin (180) through perimeter air intakes (182) around the base of the bin. The lye fountain is actually a downward flowing lye film (187) which absorbs CO₂ from the air to form sodium bicarbonate solution which exits spill-off drain (190), and enters the remainder of the Type #2 stage-1 invention system, followed by substantial stage-2 capture amplification at sea (FIGS. 6, 7).

Although one algae conversion silo appears in FIG. 12, a cluster (not shown) may be envisioned in which each lye fountain bin (180) is surrounded by four algae conversion silos (18, 90) in a non-limiting example. Remediation parks containing, e.g. 48 of these clusters may be envisioned in a non-limiting example of high capacity outdoor air capture. Global proliferation of such remediation parks, e.g., 20,000-200,000 parks in a non-limiting example and coupling these parks to stage-2 invention ocean amplification (FIGS. 6, 7) will contribute to the FIGS. 1, 7 CO₂ contingency capture goal (2, 81) of 17 GtC/yr.

In FIG. 12, the lye fountain bin (180) houses a large, slow-rotating (e.g. ˜9 rpm in a non-limiting example) air auger (181) which draws CO₂-laden air into the bin at perimeter intakes (182) located all around the base of the bin. The auger (181) pushes the air spirally up through the bin where it exhausts at the stripped-air exits (183). The air auger (181) drive shaft is hollow in a preferred embodiment. In one preferred non-limiting embodiment, the hollow shaft houses a smaller, higher speed auger (not shown) which draws lye solution from reservoir (184) up into the hollow shaft (185) and propels it internally to the top of the drive shaft where it spills out (186) onto the upper extent of the large slow-moving air-auger blades (181). The lye solution spreads over the auger blades into a lye film (187) of very high surface area which runs down the blades as a film, flowing counter to the rising air column being pushed spirally upward by the blades. Gravity draws the lye film (187) spirally downward over the blades as blade rotation pushes the air upward. This is an efficient, high surface area film reactor in which the rising flow of air (182→183) interacts with the downward (film) counter-flow (187) of lye solution. The downward flowing lye film (187) absorbs CO₂ from the air as it passes through the bin and the lye film may be quantitatively converted to sodium bicarbonate solution which spills off the bottom of the auger blades at 188, hits a sloping false bottom (189) in the bin, and exits via the indicated sodium bicarbonate (NaHCO₃) drain (190). From there, the sodium bicarbonate enters the remainder of the stage-1 invention system as in FIG. 13 followed by substantial stage-2 capture amplification at sea (FIGS. 6, 7).

In Type #3 (NaHCO₃ starter path) embodiments of the multi-stage naturally amplified global scale carbon dioxide capture system, FIG. 13 illustrates that any generic source (200) of carbonate or bicarbonate solution resulting from CO₂ capture may be processed by subsequent invention closed-system acidification (131-142) of the bicarbonate solution and infusion of the re-released carbon dioxide (138) into invention stage-1 bioreactors (algae conversion silos (18, 90) where it feeds algal blooming to produce the stage-1 seed (20) for stage-2 ocean-amplified blooming (FIGS. 6, 7).

FIG. 8 shows some of the internal workings of one possible embodiment of the stage-1 algae conversion silo (18, 65, 90) from FIGS. 3-6, 9, 10, 12, and 13. The FIG. 8 algae silo (90) also houses a fountain, but in this case, FIG. 8 shows that the lower blade extent of a tapered rotating auger (95) is immersed in a pool of suspended seed algae (94). This auger (95) rotates faster (e.g. 50 rpm in a non-limiting example). With its lower blade extent (95) immersed and clockwise rotation (in a top view perspective—not illustrated), the 50 rpm auger continuously lifts algae suspension out of the pool and slings it off the edges of the auger blade to form a helical sheet fountain of watery algae suspension through a majority of the silo headspace.

The helical fountain sheets are optically thin, so light penetration is enhanced and exceptionally high seed levels of algae may be employed without encroaching on light penetration (algal suspension opacity) limits. With optical thinning induced by the sheet fountain, up to 15% solids may be tolerated as a seed level, which is generally higher than prior art algal bioreactors. Because algal bloom rates are strongly dependent on seed level, with the bloom rate accelerating nonlinearly with increases in seed level, prodigious algal blooming will characterize this stage-1 bioreactor—potentially blooming at elevated rates.

In one preferred embodiment, and referring to FIG. 8, the stage-1 invention bioreactor (algae conversion silo (90)) will convert high levels of headspace CO₂ (91) to algae (20) by accelerated photosynthesis, the algae conversion silo (90) comprising a liquid pool (94) seeded with a starter seed of algae at the bottom of a silo, and a silo headspace continuously infused (90, 92) with elevated levels of CO₂ from the FIGS. 3-5 and 9-13 invention embodiments. Depending on the invention configuration (among FIGS. 3-5 and 9-13), concentrated CO₂ in FIG. 8 may be injected into a headspace above the algae pool (94) either via port 91 or port 92. It should be noted that the FIG. 8 algae conversion silo inner works detail applies equally to all of the invention algae conversion silos, including the silos (18, 65, 90) of FIGS. 3-6 and FIGS. 9-13. For the invention embodiment configurations of FIGS. 9-13, port 91 of FIG. 8 would be a headspace recirculation outlet port through which headspace gases are pulled out of the silo (e.g. by a fan (not shown)) and circulated through the gas-liquid separator (139) headspace of FIGS. 9, 10, 12, and 13 (where the circulating headspace gases (138, 140, 142, 143) pick up CO₂, released by acidifying sodium bicarbonate solution, and carry it into the silo headspace at port 142 (FIG. 8, port 92). In that case (FIGS. 9, 10, 12, and 13) CO₂ released in the gas-liquid separator (139) would be injected into the FIG. 8 silo head space recirculation input port (92).

Continuing with the FIG. 8 invention bioreactor embodiment summary, the algae conversion silo (90) further comprises artificial lighting (96) and a vertically-oriented rotating auger (95), the auger with its lower blade extent (95) immersed in the seeded liquid pool (94), in which the rotating auger (95) lifts a watery suspension of the seed algae from pool and slings at least 1 helical sheet fountain of the suspension of seed algae from edges of the auger blade producing increased surface area for headspace carbon dioxide exposure (to thin helical fountain sheets of seed algae suspension) and also producing an optical thinning effect (thin sheet(s) of seeded suspension slung in a helical pattern through the headspace) enhancing light penetration into the thin fountain sheets to activate photosynthesis in the suspension as it falls back into the pool or runs back down the silo walls into the pool, and in which the enhanced light penetration from optical thinning enables use of elevated seed levels without encroaching on optical opacity limits, and in which the enhanced light penetration enables seeding higher on an upward-bending nonlinear growth curve and corresponding acceleration of algal blooming by significantly accelerating photosynthesis and resulting in significantly accelerated algal blooming and carbon dioxide capture, and in which enhanced light penetration also allows blooming to develop significantly further before opacity limits are reached, and in which the liquid pool (94) comprises a suspension of algae, which in a nonlimiting example would be a salt-water suspension of coccolithophore and/or siliceous diatom or other high density marine seed algae and micronutrients, plus an optional pH buffer stabilizing the liquid pool nominally at pH 8.32 against acidification by carbonation from the high CO₂ gas levels and/or CO₂ partial pressures in the headspace above the pool.

In the stage-1 bioreactor silo (90), buffering the algae suspension (94) at approximately pH 8.32 in a non-limiting example may be achieved in one non-limiting example by adding a mixture of disodium phosphate and monosodium phosphate to the pool (94) in a mole-ratio of approximately thirteen-to-one, respectively, in which the phosphate buffering components also double as at least one component of photosynthesis micronutrients to support algal blooming. Other acid-base mixture pairs such as a borate system may also be envisioned that would buffer the pool to a desired pH range.

The approximately 13-to-1 buffered mixture of phosphates or a borate buffer or a combination buffer will result in a pH of approximately 8.32. If desired, that pH may be achieved by supplementing other mixture ratios of the phosphates with additional acids or bases to convert the phosphates to an equivalent 13-to-1 ratio of the more basic phosphate form (nearest to pKa₂) and its conjugate acidic form. Phosphate buffering at pH 8.32 may further be aided by addition of sodium bicarbonate, as needed.

In one embodiment of stage-1 invention bioreactor (algae conversion silo (90)), artificial lighting (96) shines through the helical sheet fountain at programmed intervals. This may be either CW or modulated lighting to produce accelerated blooming from modulation (foreshortened light/dark cycles), the accelerated blooming acceleration deriving from the temporal relationship of photosynthetic light reactions and dark reactions. The dark reactions are important. Both light and dark cycles are required, but natural bloom rates are limited to the 24 hour solar cycle. Artificial lighting can shorten that cycle and accelerate blooming simply by shortening the duration of each light cycle, and immediately beginning a dark cycle, which is also of reduced duration. In one non-limiting embodiment, the light intensity of the foreshortened light cycle may be increased to advantage in accelerating blooming. In addition, using artificial lighting (96) means that photosynthetic quantum efficiency need no longer be limited to the 11% value typical of the solar spectrum. In one preferred (but non-limiting) invention embodiment, light emitting diodes may be used at (96) with their emission wavelengths being optimally selected to maximize the quantum efficiency of high-density marine algae photosynthesis and/or calcification. For some algae this would mean using a majority of red photodiodes with a minority fraction of the diodes being blue (and perhaps none being green or yellow) in a non-limiting example. The balance of red and blue photodiodes may be adjusted to maximize photosynthetic quantum efficiency (specifically for coccolithophore blooming, if appropriate) to values anticipated to be in the range of 40-70%. Other wavelengths may also be selected to maximize the blooming rate of any given algae species and still be within the scope of invention.

Further acceleration of bloom rates in invention stage-1 bioreactors using a recirculating headspace oxygen removal system (119) to remove headspace oxygen (in a non-limiting example through an O₂ permeable tubular membrane (116) with concentric counter-flow of nitrogen (113) to sweep oxygen away from the far side of the membrane as it permeates the membrane walls (116)) as fast as O₂ is produced by photosynthesis. Lowering the headspace oxygen level in the invention stage-2 bioreactors may accelerate algal blooming rates. Dissolved oxygen removal from the invention algae pool may be effected by continuous bubbling of nitrogen (not illustrated) through the silo pool (94), driving the dissolved oxygen into the silo headspace where it is removed by the headspace oxygen removal system (110-116). Lowering the dissolved oxygen level in the algae silo pool (94) will further accelerate algal blooming rates.

In a non-limiting preferred embodiment example, once stage-1 bioreactor (90) blooming reaches 15% solids in the algae pool (94), a smaller transfer auger (not shown) is turned on to remove algae suspension from a lower extent (99) of the bioreactor algae pool, as fast as it blooms, thereafter. This maintains a constant 15% algae concentration in pool (94) (e.g. 15% suspended solids) as blooming proceeds continuously. It also maintains a self-reseeding condition in the reactor at a constant 15% solids seed level, which is very high on the nonlinear growth curve, stimulating prodigious algal growth, which is also continuously removed (as fast as it blooms) and continually reseeded. The stage-1 invention is therefore a continuous algal bioreactor with prodigious bloom rates. Mechanical shearing action of the large vertical rotating lift auger (95) and also of the smaller transfer auger will prevent colonization (agglomeration) of the rapidly blooming algae, so that the continuous harvest algae remains free-flowing and smoothly exits the bioreactor at 99, removed via the transfer auger. Removal is to an adjacent separation tank (101, 100). As the 15% algae suspension is continuously removed by the transfer auger, replenishment sea water (21) is provided to maintain the silo pool (94) at a constant liquid level and replenishment nutrients and pH buffer are also provided (21) to maintain constant bioreactor blooming conditions at a very high level.

The separation tank (100) is relatively large diameter to cause a significant reduction in flow velocity at the same flow rate as 101. This velocity reduction is important, because it suddenly offers the tiny algae (e.g. 2 μm in diameter in a nonlimiting E. huxleyi example) an opportunity to swim against the current, if they so desire. What is needed next is a reason for the algae to swim against the current so that they will concentrate in the upper end of the separation tank. That impetus is provided by tank (100) and its main downward flow path being dark and essentially devoid of both CO₂ and nutrient, whereas an attractant light beam (beacon 106, 107) is positioned within the mouth of a harvest exit tee (105) located near the upper extent of tank (100). With the main separation tank volume (100) and path (101-102) being essentially devoid of light, and with the flow velocity significantly reduced at large tank diameter, the algae may swim against downward current (101→102)—swimming upward instead toward the attractant beacon (107) and illuminator globe (106) supplied at the mouth of the harvest exit tee (105). The exit tee and harvest exit path (105→20) are smaller in diameter again and, even though the exit path (105→20) flow rate is low, this diameter reduction raises flow velocity (relative to path 101→102) enough that any algae which appear at the mouth of the exit tee (106, 105) will be sucked into harvest exit flow path (105). Marine algae may be continuously harvested as ocean seed at the harvest output of the silo (20). The bioreactor is continuous, self-concentrating, and will promote prodigious algal blooming at output (20). About 85% of the algal bloom will continuously exit via the harvest path (15) in a nonlimiting example, with about 15% recirculating via path (102-104). Any dead algae will sink and may be periodically removed at (109).

The FIG. 8 invention bioreactor concept may be compartmentalized into bloom, oxygen removal, and separation/concentration/harvest sections. The bloom section would be section 90 (comprising items 91-99 and 118). The oxygen removal section (119) would comprise items 110-116. The separation/concentration/harvest section would comprise items 101-109 and 20. One algae silo (18) embodiment is depicted. Many others may be envisioned substituting different bloom section (90) designs to function with the 101-109, 20 separation/concentration/harvest section, or a different harvest concept such as an adjacent recirculating settling tank to replace the FIG. 8 illustrated separation tank (100) may be employed and still be within the scope of the FIG. 1, FIGS. 3-13 invention system.

Using a non-limiting preferred FIG. 8 embodiment example with a 16-33 foot silo diameter, a 7-15 foot diameter tapered auger (95) rotating at 50 rpm, and assuming a quantum efficiency of 44%, we calculate (and estimate) a stage-1 production capacity of 1000 lb of coccolithophore algae/day/silo in the Type #1 FIG. 3 CC coal-fired power plant invention embodiment. That would amount to 1,200 lb of stage-1 daily CO₂ conversion (to algae) per silo. For a 500 mega-watt power plant, a 400 acre farm with an array of 82×82=6,724 of algae conversion silos (18, 90) would then be required to convert all of the power plant (SCF) CO₂ to coccolithophore algae and the 6,724 silos' operation (lighting (96), motors (98), temperature control, etc.) would consume approximately 34% of the total daily power plant energy production.

Since artificial lighting (96) is employed and a holding tank is provided for SCF-CO₂ (FIG. 3, item 13), electrical power to operate the algae conversion silos need not be used at peak grid demand hours. Silos (18, 90) may instead divert their highest operational demand to (grid) off-hours in order to reserve maximum peak power plant usage for grid customers. By consuming more energy in the power plant off hours and less energy during (grid) peak hours, the algae silos need not encroach on the power available to grid customers, and grid sales may remain essentially unaffected for the power company. In fact, power company sales will increase with the algae silo farms buying the available off-hours excess capacity. Power plants may thus operate near maximum capacity 24 hours/day instead of just during peak grid demand hours. Reasonably assuming that multi-national governments will eventually pay a subsidy for the algae silo farm programs to impart an 700% (multi-stage, including FIGS. 6, 7) negative carbon footprint (whole-earth carbon accounting) to CC clean-coal or GG gas-fired combustion to help reverse global warming, or assuming that grid customers will eventually foot the extra 34% increase in power cost, or assuming a reasonable and fair balance of government subsidy and grid customer price increases (e.g. 17% subsidy and 17% price increase in a non-limiting example) or a program taking the cost of algae conversion with CC coal power plants and redistributing it among all coal power plants and/or all gas-fired power plants, or a combination of the above is established to cover the algae silo farm power consumption, utility company profits may be maximized.

In yet more embodiments, carbon dioxide separation and concentration may be achieved by any prior-art means from any CO₂ source with the captured and concentrated CO₂ being directly coupled to FIG. 5 invention stage-1 bioreactors (65) subsequently leading to FIGS. 6, 7 invention stage-2 ocean amplification.

FIG. 6 illustrates that up to 3 GtC/yr of seed algae produced by land-based stage-1 (70) algae conversion silos (65) may be transported to seaports where it will couple to invention stage-2 (71), which is more of a process invention, in which “algae +nutrient” (versus the essentially universal (albeit unsuccessful) prior-art practice of seeding “nutrient-only”) are seeded (75) into the oceans (76—see also FIG. 7, items 80, 82) under heretofore unequalled (either in prior art or in nature) rapid blooming conditions which collectively favor an unprecedented maximum ocean blooming capacity of 14 GtC/yr in 12 monthly blooms/yr. This will eventually yield an unprecedented 15× invention ocean-amplified CO₂ capture, with each 1 ton of stage-1 land-based invention CO₂ capture (and invention bioreactor algal conversion) triggering an additional 14 tons of CO₂ capture via invention-system-induced amplified ocean blooming. When 14 GtC/yr of invention system stage-2 amplified ocean capture is added to up to 3 GtC/yr of invention system stage-1 land-based capture, the overall stage-2 invention system capture curve 81 (see also FIG. 1, item 2) may finally be achieved, driven (triggered) by the smaller invention system ocean seeding curve (80, 82). Humans need only provide the invention system seed curve (80, 82), under heretofore unmatched favorable conditions, and the oceans will then do the required (invention-system-amplified and accelerated) “heavy lifting” of capture curve 81 (see also FIG. 1, item 2, which target would be met by FIG. 7, item 81).

To understand the “heretofore unmatched favorable conditions” required for successful invention 15× stage-2 process amplification, classic prior art limitations must be overcome, including low natural (and prior art) starter seed concentration (FIG. 2), the buoyancy of prevailing natural strains dominating the majority of conventional ocean blooming (including natural and all prior art attempts) and which buoyancy prevents repeated monthly blooming cycles as dead algae form a persistent floating light penetration block in the photic zone, the voracious appetites of currently overpopulated (and starving) zooplankton grazers which can easily devour all of conventionally available (natural) starter seed—before it has a chance to bloom significantly, the limited available nutrient owing to stratification of warm seas which creates a thermocline that prevents upwelling of deep water nutrients from replenishing top water (photic zone) depletion (FIG. 2) following initial (natural) springtime algae blooming, and the devastating post-bloom anoxia which may result from secondary bacterial blooming following the death of large algae blooms fed by agricultural runoff into coastal waters. Invention-optimized stage-2 ocean blooming conditions will overcome each of the above mentioned classic prior art (and natural) barriers and limits which would otherwise prevent achievement of the massive CO₂ capture amplification required for the FIG. 7 ocean seeding curve (80, 82) to successfully produce the required capture curve (81).

To summarize invention-optimized stage-2 ocean blooming conditions, the low starting point of FIG. 2 will be remedied by invention seeding “algae+nutrient” into the oceans, versus the prior-art practice of seeding “nutrient only”. The invention stage-2 process will seed higher on the upward-bending nonlinear growth curve than prior-art or natural ocean conditions, and this higher seed level will accelerate ocean blooming—essentially blooming locally to the light penetration (algal bloom opacity) limits within only 2 weeks (each month). The buoyancy problem will be remedied by exclusively seeding high-density, fast-sinking coccolithophore algae (with heavy calcium carbonate exoskeletons) or heavy siliceous diatoms, selectively produced in FIG. 5, 6 (item 65) stage-1 bioreactors, and seeding them into the oceans at local levels which are substantially higher on the growth curve than the natural buoyant algae strains, so that the heavy coccolithophore or siliceous diatoms virtually dominate the stage-2 ocean blooms. Invention-controlled nutrient selection may also enhance species selective bloom dominance at sea. The high-density coccolithophore or siliceous diatoms will then bloom rapidly to the light penetration (algal opacity) limit, die, and then sink rapidly (post mortem) each month, thereby clearing the photic zone (eliminating natural and prior art light blocks) in preparation for the next month's bloom cycle. The problem of overpopulated voracious grazers devouring seed before it has a chance to bloom will be circumvented by invention front-end seeding of 3 GtC/yr (FIG. 7, item 80) which exceeds annual estimates of currently overpopulated grazer appetites which are 2 GtC/yr and is anticipated to satiate grazer appetites so that the grazers leave a net 1 GtC/yr of seed uneaten and therefore available to seed the amplified blooming of curve 81.

In all of its various FIGS. 3-5 and 9-13 invention system embodiments, should collectively exhibit sufficient stage-1 output algal-seed capacity (e.g. up to 3 GtC/yr in a non-limiting example) of a globally-proliferated multiplicity of the invention stage-1 bioreactors (18, 65, 90) to exceed the appetites (e.g., 2 GtC/yr) of zooplankton grazers when seeded into the ocean, such that the grazers will eat only an approximate ⅔ fraction of the ocean-dispersed stage-1 seed (FIG. 7 curve segment (80)) before it blooms (at sea) in stage-2 (81), leaving a significant net (uneaten) ⅓ fraction (e.g., 1 GtC/yr) of the ocean-dispersed stage-1 seed (82) to bloom with up to 15× invention-system-induced compound ocean amplification in stage-2, yielding a 14 GtC/yr oceanic bloom (ocean fraction of curve 81) in a non-limiting example of the invention system.

The stage-2 invention system 3 GtC/yr front end seed “bump” (80) may only be needed for several years as predators (of grazers) re-proliferate their numbers (currently decimated by commercial over-fishing) in response to the rising edge of curve (81) which will feed massive (15× amplified) quantities of algae at the bottom rung of the marine food chain. Prodigious bottom-rung feeding will stimulate the entire marine food chain to re-proliferate, restoring populations of predators who will quickly eat the grazer populations back down, thereby eliminating the need for continued, 3 GtC/yr seeding (80), such that seed levels may then be diminished to about 1 GtC/yr (82) after the initial seed bump (80). At this point, the diminished grazer populations will eat from the invention-system-induced amplified 14 GtC/yr bloom, rather than devouring the seed before it has a chance to bloom. Populations of grazer and predator are expected to surge back and forth multiple times as the natural population balance is restored. This back and forth surging is represented by the series of spikes anticipated on the leading edge of the amplified ocean capture curve (81).

Nutrient depletion in modern, warm, stratified seas will be invention-system remedied by adding metered doses of nutrient with each seeding. Metered doses will support localized maximal coccolithophore or siliceous diatom algal blooming until it reaches light penetration (algal bloom opacity) limits within ˜2 weeks, and then nutrient will run out. The bloom will quickly starve and die as it approaches the light penetration limit. Blooms will thus not be overfed, and natural buoyant strains of algae won't bloom significantly in comparison to the selective prodigious blooming of high density, fast-sinking invention system coccolithophore or invention siliceous diatom algae seed.

Undesirable post-bloom anoxia which ordinarily follows the death of natural (or agricultural runoff induced) algal blooms partially depletes dissolved oxygen (DO) to depths of 300-800 meters in the open sea and fully depletes DO to the sea floor in shallow coastal waters. This kills fish and other marine life in coastal waters and gives rise to a popular perception that large algae blooms at sea are “bad”.

To prevent post-bloom anoxia in the invention stage-2 amplified ocean blooming, species specific bloom dominance will ensure only heavier-than-water coccolithophore or siliceous diatoms contribute to the blooms. These heavier-than-water algae will sink rapidly (post mortem) to the bottom of the open sea, where they will be preserved by low deep sea floor temperatures in the range of 0-4 degrees C. until they are buried by 1 mm/year sedimentation of marine “snow”. Low temperature deep-sea floor preservation and relatively rapid burial, plus armored exoskeletal coverage of these heavier-than-water algae comprise the prime avoidance hypothesis advanced for this invention system for stage-2 seeding in the open seas (deep water) which would expect to be done monthly for 45 years (see FIG. 1) over about 70% of the ocean surfaces.

In coastal waters, the monthly death of each stage-2 bloom will be immediately followed by forced local re-aeration of seeded areas to a depth of within 5 meters of the sea floor in shallow coastal waters. This invention forced re-aeration will effectively prevent post-bloom anoxia in coastal waters and will greatly benefit coastal marine life. With massive bottom rung feeding in both coastal and open sea water, re-proliferation of marine populations which are currently decimated by commercial over-fishing should occur. Burgeoning populations of marine life (last seen in the 18^{th} and 19^{th} centuries) may be restored within the first 10 years of curve 81 (FIG. 7); especially if a permanent whaling ban and a temporary moratorium on commercial wild-capture fishing were also to be established and enforced (with seafood markets being replenished by an expansion in commercial fish farms (which currently account for 42% of seafood market supply)). In that case, fish farming only need expand by 2.4× in order to meet seafood market demand in the event of a wild-capture commercial fishing moratorium.

Invention benefits will extend well beyond the climate stabilization summary illustrated in FIG. 1. FIG. 1 illustrates that atmospheric CO₂ may be expected to return to the preindustrial level of 280 ppm by 2075. In addition, ocean acidification will disappear as ocean pH automatically rises to 8.33 with the reduction in atmospheric CO₂ leading to lower carbonation levels in the oceans. Restored populations of marine life will be an added benefit.

In addition to the invention stage-1 bioreactors contributing significantly to climate stabilization, other applications for stage-1 high capacity algal production will include silage, animal feed, feed supplements, fertilizer, food for fish and seafood farming involving species of fish or mollusk which directly feed on algae, and bottom-rung food for fish farming involving predator fish (as seafood) such as compano and cobia which feed on lower marine life (e.g, brine shrimp). In the latter case, invention high capacity algal production will feed the brine shrimp in separate tanks, raising shrimp for secondary feeding to predator fish.

Invention stage-1 algal bioreactors can also be applied to high capacity production of beneficial species of fresh water algae which, if properly selected and managed, can revitalize inland lakes and rivers by removal of nitrogen and phosphorus compounds added by agricultural runoff. Clearing major rivers of agricultural runoff will also stop coastal water harmful algae blooms (HAB's) such as the notorious “red tide” in Florida, resulting from agricultural runoff at major river delta outflows.

There may be bio-fuel applications that benefit from high capacity algal production in the invention stage-1 bioreactors. However, these won't contribute to climate stabilization since biofuel combustion returns CO₂ to the atmosphere (zero-sum-gain). Biofuels wouldn't exhibit amplification of CO₂ capture and they may finally divert resources and attention away from hydrogen fueling of transportation.

Specific invention inclusions are listed below.

1. The invention specifically includes a system for production of algae, the system comprising a CO₂ source and a bioreactor supplied with concentrated CO₂ from the CO₂ source, the bioreactor configured to encourage accelerated growth and reproduction of algae as well as to enable development of a more concentrated final algal bloom; in which optical opacity limits on seed level and bloom concentration are circumvented by an optical thinning effect which enables greater light penetration into more concentrated algae suspensions; wherein the greater light penetration enables higher level initial seeding or inoculation of the bioreactor bloom space; wherein the higher level of initial seed accelerates blooming as a result of starting higher on a nonlinear algal growth curve; and in which a normally inaccessible upper section of the nonlinear algal growth curve is conventionally inaccessible owing to optical opacity of concentrated algal suspensions; and in which the normally inaccessible upper section of the nonlinear growth curve is rendered accessible by the optical thinning effect which enables light penetration into optically thinned suspensions of concentrated algae.

2. The invention further includes the system of preceding section 1, wherein the optical thinning effect is produced by slinging an algae suspension as thin watery sheets off the perimeter edges of a rotating auger blade which lifts algae suspension out of a pool, elevates the suspension, and slings it outward by centrifugal force to form optically thin watery sheets, and wherein optical thinness of the slinging sheets enables improved optical penetration by rays from a light source shining through the slinging sheets.

3. The invention further includes the system of preceding section 1, wherein the optical thinning effect is produced by spraying, misting, or aerosolizing an algae suspension as droplets and particles to form optically thin sprays, mists, or aerosols, wherein optical thinness of the algal sprays, mists, or aerosols enables improved optical penetration by rays from a light source shining through the sprays, mists, or aerosols.

4. The invention further includes the system of preceding section 1, wherein the optical thinning effect is produced by directing a flow of an algae suspension through an annular space occurring between two axially concentric tubes, and wherein the annular space occurs between the outside diameter wall of the innermost tube of the two axially concentric tubes and the inside diameter wall of the outermost tube of the two axially concentric tubes, wherein the annular space is less than 50 mm thick, and wherein the optical thinness of the flow of algae suspension within the annular space enables improved optical penetration by rays from a light source shining through the flow of algae suspension contained within the optically thin annular space.

5. The invention further includes the system of preceding section 1, in which the algae suspension from the bioreactor proceeds to a flow-through separation tank after blooming, wherein the flow velocity of algae suspension through the separation tank is reduced, at constant flow rate, by means of enlarged tank diameter, and wherein the reduced flow velocity is low enough to permit algae that have flagella or other motility means to swim effectively against the flow current when presented with an upstream or side-stream attractant, and wherein the direction of algal swimming is toward the attractant, and wherein algal swimming toward the attractant produces a concentrating effect on the algal suspension, and wherein the concentration of algae proximal to the attractant is made higher by the concentrating effect than the concentration of algae at points located progressively downstream from the attractant and still within the main flow of the flow-through separation tank.

6. The invention further includes the system of preceding section 5, wherein the separation tank contains a main flow exit port and a secondary exit port which is designated as a harvest exit tee, and wherein the attractant is located at a position proximal to the mouth of the harvest exit tee, and wherein the mouth of the harvest exit tee is sufficiently narrow to raise the harvest exit flow velocity to exceed the capacity for algae to swim against the harvest exit current, wherein algae swimming toward the attractant from the main separation tank are sucked into the harvest exit tee upon reaching the attractant, and wherein the harvest exit tee outflow leads to an algal harvest output port, wherein the concentration of algae harvested at the harvest output port is higher than the concentration of algae entering the separation tank, and wherein the main flow of the flow through exit tank at points downstream of the attractant and having bypassed the harvest exit tee contains a reduced concentration of algae, relative to the concentration of algae entering the separation tank, and wherein the main flow of the flow through exit tank having bypassed the harvest exit tee exits the separation tank through the main flow exit port, and wherein flow exiting the main flow exit port is recirculated to the original bioreactor.

7. The invention further includes the system of preceding section 6, in which the attractant is one or more attractants selected from among a group of attractants consisting of a light source, a nutrient source, a nutrient source, a carbon dioxide source, an attractive water temperature, and an attractive water pH, and wherein the rest of the separation tank is dark and relatively devoid of the chosen attractant or combination of attractants.

8. The invention further includes the system of preceding section 1, wherein the CO₂ source is a methane (or natural gas) reformation reactor.

9. The invention further includes the system of preceding section 8, wherein the methane (or natural gas) reformation reactor is a steam cracker with stages of the steam reactor operating at two different temperatures that are optimized for hydrogen production from natural gas.

10. The invention further includes the system of preceding section 1, wherein the CO₂ source provides a concentrated flow of CO₂ gas.

11. The invention further includes the system of preceding section 10 which further comprises a CO₂ storage module.

12. The invention further includes the system of preceding section 11, wherein the CO₂ storage module includes a CO₂ liquefier.

13. The invention further includes the system of preceding section 1, wherein the bioreactor comprises an artificial light source.

14. The invention further includes the system of preceding section 4, wherein the light source is axially positioned proximal to the axial center-line of the innermost tube of the two axially concentric tubes, and wherein rays of light from the light source shine radially outward through the annular space and the flow of algae contained within the annular space.

15. The invention further includes the system of preceding section 1, wherein the bioreactor comprises a CO₂ inlet for the introduction of concentrated CO₂ gas.

16. The invention further includes the system of preceding section 1, wherein the heavier-than-water algae comprise an exoskeleton or protective coccolith plates.

17. The invention further includes the system of preceding section 16, wherein the heavier-than-water algae comprise at least one of a coccolithophore or a siliceous diatom algae.

18. The invention further includes the system of preceding section 1, wherein the CO₂ source and the bioreactor are in fluid communication.

19. The invention further includes a system for production of algae, the system comprising a hydrocarbon cracking reactor configured to generate a stream of concentrated CO₂ byproduct; and a bioreactor configured to produce heavier than water algae, the bioreactor supplied, at least in part, with CO₂ from the stream of concentrated CO₂ byproduct; and wherein the hydrocarbon cracking reactor produces H₂ as its main product.

20. The invention further includes the system of preceding section 19, wherein the hydrocarbon cracking reactor is a methane cracking reactor.

21. The invention further includes the system of preceding section 20, wherein the methane cracking reactor is a steam cracker with stages of the steam reactor operating at two different temperatures that are optimized for hydrogen production from natural gas.

22. The invention further includes the system of preceding section 19, wherein the hydrocarbon cracking reactor is a coal-gasification reactor in which partial oxidation (with O₂) converts coal to syngas—a mixture of CO and H₂; wherein the CO is further converted to CO₂ byproduct in a water-gas shift reaction with low temperature steam, and wherein the coal-gasification reactor produces H₂ as its main product.

23. The invention further includes the system of preceding section 19, wherein the hydrocarbon cracking reactor is an oil-gasification reactor in which partial oxidation (with O₂) converts oil to syngas—a mixture of CO and H₂; wherein the CO is further converted to CO₂ in a water-gas shift reaction with low temperature steam, and wherein the oil-gasification reactor produces H₂.

24. The invention further includes the system of preceding section 19, which further comprises a CO₂ storage module.

25. The invention further includes the system of preceding section 24, wherein the CO₂ storage module includes a CO₂ liquefier.

26. The invention further includes the system of preceding section 19, wherein the bioreactor comprises an artificial light source.

27. The invention further includes the system of preceding section 19, wherein the bioreactor comprises a CO₂ inlet for the introduction of concentrated CO₂ gas.

28. The invention further includes the system of preceding section 19, wherein the heavier-than-water algae comprise an exoskeleton or protective coccolith plates.

29. The invention further includes the system of preceding section 28, wherein the heavier-than-water algae comprise at least one of a coccolithophore or a siliceous diatom algae.

30. The invention further includes the system of preceding section 19, wherein the CO₂ source and the bioreactor are in fluid communication.

31. The invention further includes the system of preceding section 1, wherein the CO₂ source is a CC (carbon-capture) clean-coal-fired power plant, the power plant producing electricity as a public utility and concentrated CO₂ byproduct as a supercritical fluid (SCF-CO₂).

32. The invention further includes the system of preceding section 31, wherein the SCF-CO₂ is decompressed to concentrated CO₂ gas and introduced into the bioreactor.

33. The invention further includes the system of preceding section 1, wherein the CO₂ source is a CC (carbon-capture) gas-fired power plant, the CC power plant producing electricity as public utility and concentrated CO₂ byproduct as a supercritical fluid (SCF-CO₂).

34. The invention further includes the system of preceding section 33, wherein the SCF-CO₂ is decompressed to concentrated CO₂ gas and introduced into the bioreactor.

35. The invention further includes the system of preceding section 1, wherein the CO₂ source is a combination (CC or standard) gas-fired and CC (carbon-capture) clean-coal-fired power plant, the power plant producing electricity as a public utility and concentrated CO₂ byproduct as a supercritical fluid (SCF-CO₂).

36. The invention further includes the system of preceding section 35, wherein the SCF-CO₂ is decompressed to concentrated CO₂ gas and introduced into the bioreactor.

37. The invention further includes the system of preceding section 1, wherein the CO₂ source is a CC (carbon-capture) cement plant, the CC cement plant producing cement and concentrated CO₂ byproduct.

38. The invention further includes the system of preceding section 37, wherein the CO₂ is captured as a supercritical fluid (SCF-CO₂).

39. The invention further includes the system of preceding section 38, wherein the SCF-CO₂ is decompressed to concentrated CO₂ gas and introduced into the bioreactor.

40. The invention further includes a system for production of algae, the system comprising a CO₂ source; and a means of concentrating CO₂ from the CO₂ source; and a bioreactor supplied with concentrated CO₂ gas from the concentrating means; wherein the bioreactor is configured to encourage the rapid growth and reproduction of a heavier-than-water species of algae.

41. The invention further includes the system of preceding section 40, wherein the concentrating means produces supercritical fluid CO₂ (SCF-CO₂).

42. The invention further includes the system of preceding section 41, wherein the SCF-CO₂ is decompressed to create the concentrated CO₂ gas and introduce it into the bioreactor.

43. The invention further includes the system of preceding section 40, wherein the means of concentrating CO₂ from the source is absorbing CO₂ from the source by exposure of the CO₂ to a solution of alkali metal hydroxide (e.g. sodium hydroxide) or alkaline-earth hydroxide (e.g. calcium hydroxide) to form a CO₂ absorption product solution of alkali bicarbonate or alkaline-earth carbonate; wherein the alkali bicarbonate or alkaline-earth carbonate solution is subsequently (or downstream) acidified to re-release the captured CO₂ as concentrated CO₂ into an enclosure which is common to the bioreactor or in fluid communication with the bioreactor.

44. The invention further includes the system of preceding section 43, wherein the CO₂ source is selected from among a group of CO₂ sources consisting of a methane reformation cracker, an oil gasification syngas reactor, a coal gasification syngas reactor, a furnace flue, a water heater flue, an incinerator flue, a crematorium flue, a blast-furnace flue, a gas stove flue, a cement plant exhaust flue, a power plant exhaust flue, a refinery exhaust flue, a factory exhaust flue, and a system designed for CO₂ capture from outdoor air.

45. The invention further includes a process of ocean-amplified CO₂ capture, wherein algae plus nutrient are seeded into the ocean instead of nutrient-alone; the process comprising land-based capture of concentrated CO₂ from a land-based CO₂ source; land-based conversion of captured CO₂ to heavier-than-water marine algae in at least one bioreactor configured to encourage the rapid growth and reproduction of the heavier-than-water marine algae as ocean seed; transport of the heavier-than-water marine algae as ocean seed to seaports for ocean distribution and dispersal with added micro-nutrients in order to seed ocean-amplified blooming (further growth and rapid reproduction at sea—essentially secondary blooming on a vast ocean scale); wherein the ocean-amplified blooming occurs essentially selectively for the heavier-than-water species of marine algae by virtue of the heavier-than-water marine algae being distributed, dispersed, and seeded into the ocean water at higher levels than existing natural buoyant ocean algal strains, the higher levels selectively accelerating ocean blooming rates of the heavier-than-water marine algae by virtue of seeding the ocean higher than normal on an upward-bending nonlinear algal growth curve and producing a species-selective dominance of the ocean-amplified bloom, and wherein the higher that the ocean blooming starts on the growth curve, the faster it proceeds, if sufficient nutrient is present or provided.

46. The invention further includes the system of preceding section 45 in which the species-selective bloom dominance is further enhanced by nutrient selection.

47. The invention further includes the process of preceding section 46 in which nutrient selection for E. huxleyi coccolithophore marine algae includes nutrients which are deficient in phosphate, wherein phosphate deficiency, while also concurrently providing other nutrients in abundance, promotes prodigious E. huxleyi growth at sea, to the exclusion of blooming by other species of marine algae.

48. The invention further includes the process of preceding section 45, wherein transport to seaport of the heavier-than-water marine algae seed occurs by flat-bed truck, flat rail car, or barge; and wherein the flat-bed truck, flat rail car, or barge carry the marine algae seed in stasis-supporting cargo containers which are transferrable by crane or other lifting means from one flat-bed transportation means to another, and wherein the cargo containers are designed to maintain conditions in support of a healthy stasis condition for the heavier-than-water marine algae seed.

49. The invention further includes the process of preceding section 48, wherein the stasis-supporting cargo containers may be loaded onto ocean freighters (ships) docked at seaports, the ocean freighters then distributing the stasis-supporting cargo containers to floating seed repositories at sea; wherefrom the stasis-supporting cargo containers may be transferred to seed dispersal boats which fan out from the floating seed repositories to disperse and dispense the heavier-than-water marine algae seed (plus micronutrients) into the ocean for ocean-amplified blooming to proceed, along with ocean-amplified CO₂ capture as the heavier-than-water marine algae bloom prodigiously at sea.

50. The invention further includes the process of preceding section 49, wherein the micro-nutrient doses are metered to support heavier-than-water ocean-amplified algal blooming up to the light penetration (algal bloom opacity) limit and then run out.

51. The invention further includes the process of preceding section 50, wherein the ocean amplified bloom dies after the metered micro-nutrient doses run out; wherein the dead heavier-than-water amplified bloom sinks rapidly, clearing the ocean photic zone before the end of each month and enabling restored light penetration into the photic zone to support another amplified bloom following the next month's seeding.

52. The invention further includes the process of preceding section 51 in which 12 blooms/year may be seeded and achieved, with each ocean-amplified bloom reaching the light penetration (algal bloom opacity) limit before it dies and sinks.

53. The invention further includes the process of preceding section 52 in which accumulated amplified ocean blooming yields 14 GtC/yr of heavier-than-water algae (correspondingly capturing 14 GtC/yr of atmospheric CO₂) globally for each 1-3 GtC/yr of seeding with land-based heavier-than-water algae seed produced by the land-based bioreactors.

54. The invention further includes the process of preceding section 51, wherein local forced re-aeration of previously seeded areas to an appropriate depth prevents post-bloom anoxia from secondary bacterial blooming.

55. The invention further includes the process of preceding section 51, wherein the seeding of amplified ocean blooming is restricted to the vast open ocean that is further out from shore, well beyond the realm of coastal waters and beyond the shallow coastal-shelf sea floor, out in the open seas where much deeper water prevails, wherein species-selective bloom dominance and rapid sinking quickly carry the dead algae below the ocean thermocline of the open seas and all the way to the deep-sea floor, wherein deep ocean temperatures at the deep-sea floor are quite low—near to zero degrees centrigade, and wherein low deep-sea temperatures preserve the dead algae and slow and/or suppress the onset of secondary bacterial action, algal decay, eutrophication, and post-bloom anoxia which would otherwise deplete ocean-dissolved oxygen, and wherein the slowing or suppression of bacterial action at low temperature at the deep-sea floor delays the onset of eutrophication and post bloom anoxia to an extent enabling ocean sedimentation, often referred to as marine “snow”, to essentially bury the dead algae before post-bloom anoxia or eutrophication can develop.

56. The invention further includes the process of preceding section 55 wherein the onset of post bloom anoxia is further delayed by calcareous exoskeletal armor plates of E. huxleyi, a preferred heavier-than-water algae for ocean amplification; and wherein delay by calcareous exoskeletal armor plating dominates dead algal blooms, owing to the species-selective bloom dominance of E. huxleyi enabled by high seed levels from land-based bioreactor seed sources, and further enabled by phosphate-depleted nutrients supplied during ocean seeding with E. huxleyi seed grown in land-based bioreactors.

57. The invention further includes the process of preceding section 53 wherein approximately 1 GtC/yr of seed triggers amplified ocean blooming of up to 14 GtC/yr of heavier-than-water algae; wherein another approximately 2 GtC/yr of seed are needed (and are provided from land-based bioreactor-produced seed) to satiate marine grazer appetites so that they leave the approximately 1 GtC/yr of seed uneaten so that it remains to trigger the amplified ocean blooming of the up to 14 GtC/yr of heavier-than-water algae and corresponding photosynthetic and/or coccolithogenic (calcification) capture of up to 14 GtC/yr of atmospheric CO₂.

58. The invention further includes the system of preceding section 1, wherein the bioreactor comprises a shallow pool of seed algae; an enclosed headspace above the shallow pool; a vertical rotating auger; and overhead artificial lighting; wherein the concentrated CO₂ is injected into the bioreactor headspace; wherein the lower blade extent of the rotating auger is immersed in the pool; wherein the rotating auger lifts algae suspension up out of the pool; and wherein the rotating auger slings algae suspension off the perimeter edges of the auger blades creating a helical fountain comprising thin watery sheets of suspended algae slinging within the bioreactor headspace; and wherein the artificial lighting shines down through the thin watery sheets; wherein an optical thinning effect of the thin watery sheets allows greater light penetration through the sheets than would otherwise be possible in the pool, owing to optical opacity limits of suspended algae in the pool; and wherein the greater light penetration enables bioreactor operation at higher algae seed levels and bloom levels than would otherwise be possible without encroaching on opacity limits in the pool; and wherein the higher seed levels accelerate algal bloom rates; and wherein the concentrated CO₂ further accelerates algal bloom rates; and wherein the increased surface area of the thin watery sheets enhances algal exposure to CO₂; and wherein the increased algal exposure to CO₂ further accelerates algal bloom rates; and wherein optical thinning enables more concentrated algal blooms to develop—beyond normal opacity limits.

59. The invention further includes the system of preceding section 46, in which the rotating auger is downward tapered from top to bottom.

60. The invention further includes the system of preceding section 58, in which the bioreactor algae pool floor is funnel-shaped.

61. The invention further includes the system of preceding section 58, in which perimeter edges of the auger blade are up-angled, rather than flat.

62. The invention further includes the system of preceding section 61, in which the extent of up-angling diminishes with vertical height on the ascending auger blade.

63. The invention further includes the system of preceding section 58, in which the rotating auger is encased in a pipe, and in which section 58 slinging action is blocked by the pipe wall; and wherein auger action is limited to lifting algae suspension to the upper extent of the bioreactor, and wherein the lifted algae suspension spills out the top of the pipe-encased auger onto the apex of a dome-topped-but-otherwise-tiered-wedding-cake-shaped nebulizer; and wherein the algae suspension spreads out into a downward flowing film over the dome-topped-but-otherwise-tiered-wedding-cake-shaped nebulizer; wherein the dome-topped-but-otherwise-tiered-wedding-cake-shaped nebulizer converts the downward flowing film of suspended algae into an aerosol or mist, or spray, and wherein the misted algae particles are exposed to CO₂ of the bioreactor headspace and to light from the bioreactor artificial lighting; and wherein the mist is optically thin and presents high surface area exposure to CO₂; and wherein optical thinness and high surface area exposure accelerate algal blooming and yield a more concentrated final algal bloom.

64. The invention further includes the system of preceding section 63, in which the dome-topped-but-otherwise-tiered-wedding-cake-shaped nebulizer is hollow and internally pressurized in the range of 5-200 psi with CO₂ from the CO₂ source, introduced from the source inlet; and wherein the outward-facing essentially vertical tiered facets of the dome-topped-but-otherwise-tiered-wedding-cake-shaped nebulizer are perforated with a multiplicity of CO₂-escape orifices; wherein pressurized CO₂ escapes through the CO₂ escape orifices to the bioreactor headspace; wherein the escaping CO₂ interrupts the downward-flowing film of algae suspension covering the dome-topped-but-otherwise-tiered-wedding-cake-shaped nebulizer; and wherein the film-interruption is of sufficient velocity and turbulence to convert suspended algae to a spray, mist, or aerosol within the bioreactor headspace, and wherein the spray, mist, or aerosol is exposed to headspace CO₂ and light from the artificial illumination.

65. The invention further includes the system of preceding section 64, in which the tiered wedding-cake structure of the nebulizer allows an unmisted fraction of the algae suspension, which missed (bypassed) each CO₂ escape orifice, to continue in a downward flowing film on a first tier essentially vertical facet until it reaches the unperforated essentially horizontal upper facet of at least a second tier; where it can repool on the essentially horizontal at least a second tier upper facet; and wherein the repooled algae suspension subsequently overflows the essentially horizontal at least a second tier upper facet and spills down as a flowing film over the perforated side of the at least a second tier of the nebulizer.

66. The invention further includes the system of preceding section 60, wherein algae is removed from the bottom of the funnel shaped pool floor essentially as fast as it blooms, wherein removal is to an adjacent separation tank; and wherein the separation tank is a flow-through tank; and wherein the flow velocity of algae suspension through the separation tank is reduced, at constant flow rate, by means of enlarged tank diameter, wherein the reduced flow velocity is low enough to permit algae that have flagella or other motility means to swim effectively against the flow current when presented with an upstream or side-stream attractant, wherein the direction of algal swimming is toward the attractant, and wherein algal swimming toward the attractant produces a concentrating effect on the algal suspension, and wherein the concentration of algae proximal to the attractant is made higher by the concentrating effect than the concentration of algae at points located progressively downstream from the attractant and still within the main flow of the flow-through separation tank.

67. The invention further includes the system of preceding section 66, wherein the separation tank contains a main flow exit port and a secondary exit port which is designated as a harvest exit tee, wherein the attractant is located at a position proximal to the mouth of the harvest exit tee, and wherein the mouth of the harvest exit tee is sufficiently narrow to raise the harvest exit flow velocity to exceed the capacity for algae to swim against the harvest exit current, and wherein algae swimming toward the attractant from the main separation tank are sucked into the harvest exit tee upon reaching the attractant, and wherein the harvest exit tee outflow leads to an algal harvest output port, and wherein the concentration of algae harvested at the harvest output port is higher than the concentration of algae entering the separation tank, and wherein the main flow of the flow through exit tank at points downstream of the attractant and having bypassed the harvest exit tee contains a reduced concentration of algae, relative to the concentration of algae entering the separation tank, and wherein the main flow of the flow-through exit tank having bypassed the harvest exit tee exits the separation tank through the main flow exit port, and wherein flow exiting the main flow exit port is recirculated to the original bioreactor.

68. The invention further includes the system of preceding section 67, in which the attractant is one or more attractants selected from among a group of attractants consisting of a light source, a nutrient source, a nutrient source, a carbon dioxide source, an attractive water temperature, and an attractive water pH, and wherein the rest of the separation tank is dark and relatively devoid of the chosen attractant or combination of attractants.

69. The invention further includes the system of preceding section 67, wherein liquid replenishment is joined to the recirculation flow leading into the original bioreactor to maintain a constant liquid level in the bioreactor pool; and wherein replenishment micronutrients are added to the pool at the same rate as they are consumed by continuous blooming of the heavier-than-water algae; and wherein replenishment CO₂ from the CO₂ source is provided to the bioreactor as fast as CO₂ is consumed in photosynthesis and/or coccolithogenesis (calcification) during algal blooming.

70. The invention further includes the system of preceding section 63, wherein algae is removed from the bottom of the bioreactor essentially as fast as it blooms, wherein removal is to an adjacent separation tank; and wherein the separation tank is a flow-through tank; and wherein the flow velocity of algae suspension through the separation tank is reduced, at constant flow rate, by means of enlarged tank diameter, wherein the reduced flow velocity is low enough to permit algae that have flagella or other motility means to swim effectively against the flow current when presented with an upstream or side-stream attractant, wherein the direction of algal swimming is toward the attractant, and wherein algal swimming toward the attractant produces a concentrating effect on the algal suspension, and wherein the concentration of algae proximal to the attractant is made higher by the concentrating effect than the concentration of algae at points located progressively downstream from the attractant and still within the main flow of the flow-through separation tank.

71. The invention further includes the system of preceding section 70, wherein the separation tank contains a main flow exit port and a secondary exit port which is designated as a harvest exit tee, wherein the attractant is located at a position proximal to the mouth of the harvest exit tee, wherein the mouth of the harvest exit tee is sufficiently narrow to raise the harvest exit flow velocity to exceed the capacity for algae to swim against the harvest exit current, wherein algae swimming toward the attractant from the main separation tank are sucked into the harvest exit tee upon reaching the attractant, wherein the harvest exit tee outflow leads to an algal harvest output port, wherein the concentration of algae harvested at the harvest output port is higher than the concentration of algae entering the separation tank, and wherein the main flow of the flow through exit tank at points downstream of the attractant and having bypassed the harvest exit tee contains a reduced concentration of algae, relative to the concentration of algae entering the separation tank, and wherein the main flow of the flow through exit tank having bypassed the harvest exit tee exits the separation tank through the main flow exit port, and wherein flow exiting the main flow exit port is recirculated to the original bioreactor.

72. The invention further includes the system of preceding section 71, in which the attractant is one or more attractants selected from among a group of attractants consisting of a light source, a nutrient source, a nutrient source, a carbon dioxide source, an attractive water temperature, and an attractive water pH, and wherein the rest of the separation tank is dark and relatively devoid of the chosen attractant or combination of attractants.

73. The invention further includes the system of preceding section 71, wherein liquid replenishment is joined to the recirculation flow leading into the original bioreactor to maintain a constant liquid level in the bioreactor pool; and wherein replenishment micronutrients are added to the pool at the same rate as they are consumed by continuous blooming of the heavier-than-water algae; and wherein replenishment CO₂ from the CO₂ source is provided to the bioreactor as fast as CO₂ is consumed in photosynthesis and/or coccolithogenesis during algal blooming.

74. The invention further includes the system of preceding section 4, wherein algae is removed from the bottom of the bioreactor essentially as fast as it blooms, and wherein removal is to an adjacent separation tank; and wherein the separation tank is a flow-through tank; and wherein the flow velocity of algae suspension through the separation tank is reduced, at constant flow rate, by means of enlarged tank diameter, wherein the reduced flow velocity is low enough to permit algae that have flagella or other motility means to swim effectively against the flow current when presented with an upstream or side-stream attractant, and wherein the direction of algal swimming is toward the attractant, and wherein algal swimming toward the attractant produces a concentrating effect on the algal suspension, and wherein the concentration of algae proximal to the attractant is made higher by the concentrating effect than the concentration of algae at points located progressively downstream from the attractant and still within the main flow of the flow-through separation tank.

75. The invention further includes the system of preceding section 74, wherein the separation tank contains a main flow exit port and a secondary exit port which is designated as a harvest exit tee, wherein the attractant is located at a position proximal to the mouth of the harvest exit tee, and wherein the mouth of the harvest exit tee is sufficiently narrow to raise the harvest exit flow velocity to exceed the capacity for algae to swim against the harvest exit current, wherein algae swimming toward the attractant from the main separation tank are sucked into the harvest exit tee upon reaching the attractant, and wherein the harvest exit tee outflow leads to an algal harvest output port, wherein the concentration of algae harvested at the harvest output port is higher than the concentration of algae entering the separation tank, and wherein the main flow of the flow through exit tank at points downstream of the attractant and having bypassed the harvest exit tee contains a reduced concentration of algae, relative to the concentration of algae entering the separation tank, and wherein the main flow of the flow through exit tank having bypassed the harvest exit tee exits the separation tank through the main flow exit port, and wherein flow exiting the main flow exit port is recirculated to the original bioreactor.

76. The invention further includes the system of preceding section 75, in which the attractant is one or more attractants selected from among a group of attractants consisting of a light source, a nutrient source, a nutrient source, a carbon dioxide source, an attractive water temperature, and an attractive water pH, and wherein the rest of the separation tank is dark and relatively devoid of the chosen attractant or combination of attractants.

77. The invention further includes the system of preceding section 76, wherein liquid replenishment is joined to the recirculation flow leading into the original bioreactor to maintain a constant liquid level in the bioreactor pool; and wherein replenishment micronutrients are added to the pool at the same rate as they are consumed by continuous blooming of the heavier-than-water algae; and wherein replenishment CO₂ from the CO₂ source is provided to the bioreactor as fast as CO₂ is consumed in photosynthesis and/or coccolithogenesis (calcification) during algal blooming.

78. The invention further includes the system of preceding section 58, wherein a headspace oxygen removal system removes headspace oxygen as fast as it is produced by bioreactor photosynthesis during algal blooming; and wherein the oxygen removal system maintains pseudo-anaerobic blooming conditions in the bioreactor; and wherein the pseudo-anaerobic blooming conditions further accelerate bloom rates.

79. The invention further includes the system of preceding section 63, wherein a headspace oxygen removal system removes headspace oxygen as fast as it is produced by bioreactor photosynthesis during algal blooming; and wherein the oxygen removal system maintains pseudo-anaerobic blooming conditions in the bioreactor; and wherein the pseudo-anaerobic blooming conditions further accelerate bloom rates.

80. The invention further includes the system of preceding section 4, wherein a headspace oxygen removal system removes headspace oxygen as fast as it is produced by bioreactor photosynthesis during algal blooming; and wherein the oxygen removal system maintains pseudo-anaerobic blooming conditions in the bioreactor; and wherein the pseudo-anaerobic blooming conditions further accelerate bloom rates.

81. The invention further includes the system of preceding section 78, wherein the headspace oxygen removal system comprises an oxygen permeable membrane; wherein a non-oxygenated gas flows across a far side of the oxygen permeable membrane producing an oxygen deficit on the far side; wherein the oxygen deficit is the driving force for oxygen produced within the bioreactor headspace on a near side of the oxygen permeable membrane to exit the headspace by permeating the oxygen permeable membrane from the near side of the oxygen permeable membrane through the oxygen permeable membrane to the far side of the oxygen permeable membrane; and wherein the oxygen permeable membrane blocks the exit of CO₂ from the bioreactor headspace.

82. The invention further includes the system of preceding section 79, wherein the headspace oxygen removal system comprises an oxygen permeable membrane; wherein a non-oxygenated gas flows across a far side of the oxygen permeable membrane producing an oxygen deficit on the far side; wherein the oxygen deficit is the driving force for oxygen produced within the bioreactor headspace on a near side of the oxygen permeable membrane to exit the headspace by permeating the oxygen permeable membrane from the near side of the oxygen permeable membrane through the oxygen permeable membrane to the far side of the oxygen permeable membrane; and wherein the oxygen permeable membrane blocks the exit of CO₂ from the bioreactor headspace.

83. The invention further includes the system of preceding section 80, wherein the headspace oxygen removal system comprises an oxygen permeable membrane; wherein a non-oxygenated gas flows across a far side of the oxygen permeable membrane producing an oxygen deficit on the far side; wherein the oxygen deficit is the driving force for oxygen produced within the bioreactor headspace on a near side of the oxygen permeable membrane to exit the headspace by permeating the oxygen permeable membrane from the near side of the oxygen permeable membrane through the oxygen permeable membrane to the far side of the oxygen permeable membrane; and wherein the oxygen permeable membrane blocks the exit of CO₂ from the bioreactor headspace.

84. The invention further includes the system of preceding section 58, wherein the artificial lighting is intermittent, turning on and off on a schedule favoring maximal blooming rate for the heavier-than-water algae at the existing bioreactor temperature.

85. The invention further includes the system of preceding section 63, wherein the artificial lighting is intermittent, turning on and off on a schedule favoring maximal blooming rate for the heavier-thin-water algae at the existing bioreactor temperature.

86. The invention further includes the system of preceding section 4, wherein the artificial lighting is intermittent, turning on and off on a schedule favoring maximal blooming rate for the heavier-than-water algae at the existing bioreactor temperature.

87. The invention further includes the system of preceding section 84, wherein the bioreactor temperature is controlled to maintain a value favoring maximal blooming rate for the heavier-than-water algae.

88. The invention further includes the system of preceding section 85, wherein the bioreactor temperature is controlled to maintain a value favoring maximal blooming rate for the heavier-than-water algae.

89. The invention further includes the system of preceding section 86, wherein the bioreactor temperature is controlled to maintain a value favoring maximal blooming rate for the heavier-than-water algae.

90. The invention further includes the system of preceding section 58, wherein the wavelength of artificial lighting emissions is selected to favor maximal blooming rate for the heavier-than-water algae.

91. The invention further includes the system of preceding section 63, wherein the wavelength of artificial lighting emissions is selected to favor maximal blooming rate for the heavier-than-water algae.

92. The invention further includes the system of preceding section 4, wherein the wavelength of artificial lighting emissions is selected to favor maximal blooming rate for the heavier-than-water algae.

93. The invention further includes the system of preceding section 90, wherein the spectrum of artificial lighting is selected to include at least two wavelengths with emission intensities at those at least two wavelengths balanced to favor maximal blooming rate for the heavier-than-water algae.

94. The invention further includes the system of preceding section 91, wherein the spectrum of artificial lighting is selected to include at least two wavelengths with emission intensities at those at least two wavelengths balanced to favor maximal blooming rate for the heavier-than-water algae.

95. The invention further includes the system of preceding section 92, wherein the spectrum of artificial lighting is selected to include at least two wavelengths with emission intensities at those at least two wavelengths balanced to favor maximal blooming rate for the heavier-than-water algae.

96. The invention further includes the system of preceding section 58, wherein the pH of the heavier-than-water algae pool is buffered at approximately 8.32.

97. The invention further includes the system of preceding section 63, wherein the pH of the heavier-than-water algae pool is buffered at approximately 8.32.

98. The invention further includes the system of preceding section 4, wherein the pH of the heavier-than-water algae pool is buffered at approximately 8.32.

99. The invention further includes the system of preceding section 96, wherein buffering at pH 8.32 is achieved by dosing the algae pool with disodium phosphate and monosodium phosphate in a mole ratio of approximately thirteen-to-one.

100. The invention further includes the system of preceding section 97, wherein buffering at pH 8.32 is achieved by dosing the algae pool with disodium phosphate and monosodium phosphate in a mole ratio of approximately thirteen-to-one.

101. The invention further includes the system of preceding section 98, wherein buffering at pH 8.32 is achieved by dosing the algae pool with disodium phosphate and monosodium phosphate in a mole ratio of approximately thirteen-to-one.

102. The invention further includes the system of preceding section 99, wherein the mole ratio is other than thirteen-to-one and the pH is other than 8.32 during initial preparation; wherein other acids, bases, or amphoteric salts are added to readjust the actual solution concentrations of disodium phosphate and monosodium phosphate to a mole ratio of approximately thirteen-to-one via acid-base reaction; wherein the pH is thereby adjusted to approximately 8.32.

103. The invention further includes the system of preceding section 100, wherein the mole ratio is other than thirteen-to-one and the pH is other than 8.32 during initial preparation; wherein other acids, bases, or amphoteric salts are added to readjust the actual solution concentrations of disodium phosphate and monosodium phosphate to a mole ratio of approximately thirteen-to-one via acid-base reaction; wherein the pH is thereby adjusted to approximately 8.32.

104. The invention further includes the system of preceding section 101, wherein the mole ratio is other than thirteen-to-one and the pH is other than 8.32 during initial preparation; wherein other acids, bases, or amphoteric salts are added to readjust the actual solution concentrations of disodium phosphate and monosodium phosphate to a mole ratio of approximately thirteen-to-one via acid-base reaction; wherein the pH is thereby adjusted to approximately 8.32.

105. The invention further includes the system of preceding section 43, wherein the alkali metal hydroxide and/or the alkaline-earth hydroxide solution(s) are spread into an essentially downward continuous flowing film of exposed surface area, and wherein the source of CO₂ is a continuous gaseous counter-flow (essentially an upward flow) exposed to the solution film.

106. The invention further includes the system of preceding section 105, wherein the essentially downward continuous flowing solution film flows spirally downward, covering and flowing down the blade or blades of a slowly rotating vertical auger, wherein the auger is housed within a silo or bin which is marginally larger in diameter than the auger diameter, and wherein the CO₂ source is CO₂-laden outdoor air, and wherein the silo or bin has outdoor air intake ports around the base of its perimeter proximal to the lower extent of the auger blades, and wherein rotation of the auger draws outdoor air into the bin or silo at its base and lifts it spirally upward through the bin or silo, ejecting it near the top, and wherein the spirally upward moving air moves in an upward spiral counter-flow to the downward-spiraling flowing solution film, and wherein the downward-spiraling flowing solution film absorbs CO₂ from the upward-spiraling counter-flow of air, and wherein the downward-flowing film solution is converted to alkali bicarbonate or alkaline-earth carbonate solution by absorbing the CO₂, and wherein the bicarbonate or carbonate solution spills off the bottom of the auger blades onto a surface which drains to an exit drain from the silo or bin.

107. The invention further includes the system of preceding section 105, wherein the essentially downward continuous flowing film is formed by a rising flow of alkali hydroxide or alkaline-earth hydroxide solution being directed upward through a vertical standpipe housed within a cylindrical chamber, and wherein the rising flow of solution continuously overflows the top of the vertical standpipe and spills down the exterior wall of the standpipe forming a downward-flowing film of solution on the exterior surface of the standpipe, flowing off the bottom of the standpipe exterior onto a chamber floor surface which is continuous with the exterior of the standpipe, and wherein the floor surface drains into an exit drain from the chamber, and wherein the CO₂ source is a gaseous upward counter-flow of CO₂-laden gas which enters the chamber tangentially at a point higher than the exit drain, and wherein the upward counter-flow of CO₂-laden gas is a laminar counter-flow, a turbulent counter-flow, or a vortex counter-flow encircling the standpipe and rising concentrically around it in the annular space between the standpipe and the chamber wall, and wherein the upward counter-flow of CO₂-laden gas exits the chamber near its upper extent, and wherein the upward laminar counter-flow, turbulent counter-flow, or vortex counter-flow of CO₂-laden gas is exposed to the downward-flowing film of alkali hydroxide or alkaline-earth hydroxide solution, and wherein CO₂ in the upward laminar counter-flow, turbulent counter-flow, or vortex counter-flow of gas is absorbed by the downward-flowing solution film, and wherein absorbing CO₂ causes the downward-flowing solution film to be converted to alkali bicarbonate or alkaline-earth carbonate solution by the time it reaches the lower extent of the standpipe exterior, and wherein the alkali bicarbonate or alkaline-earth carbonate solution exits the exit drain.

108. The invention further includes the system of preceding section 1, in which heavier-than-water algae from the bioreactor proceed to an adjacent settling tank after blooming, and in which settling tank conditions are maintained that do not encourage algae to swim against a current, and in which the heavier-than-water algae instead sink toward a funnel shaped harvest exit port at the bottom of the settling tank, and in which optional recirculation of clarified liquid near the top of the settling tank is provided back to the main bioreactor, with top-water clarification occurring as the algae sink to the funnel shaped bottom, and in which a concentrating effect is achieved via sedimentation of the sinking algae prior to their exit at the harvest exit port.

109. The invention further includes the system of preceding section 60, in which heavier-than-water algae from the bioreactor proceed to an adjacent settling tank after blooming, and in which settling tank conditions are maintained that do not encourage algae to swim against a current, and in which the heavier-than-water algae instead sink toward a funnel shaped harvest exit port at the bottom of the settling tank, and in which optional recirculation of clarified liquid near the top of the settling tank is provided back to the main bioreactor, with top-water clarification occurring as the algae sink to the funnel shaped bottom, and in which a concentrating effect is achieved via sedimentation of the sinking algae prior to their exit at the harvest exit port.

110. The invention further includes the system of preceding section 63, in which heavier-than-water algae from the bioreactor proceed to an adjacent settling tank after blooming, and in which settling tank conditions are maintained that do not encourage algae to swim against a current, and in which the heavier-than-water algae instead sink toward a funnel shaped harvest exit port at the bottom of the settling tank, and in which optional recirculation of clarified liquid near the top of the settling tank is provided back to the main bioreactor, with top-water clarification occurring as the algae sink to the funnel shaped bottom, and in which a concentrating effect is achieved via sedimentation of the sinking algae prior to their exit at the harvest exit port.

111. The invention further includes the system of preceding section 4, in which heavier-than-water algae from the bioreactor proceed to an adjacent settling tank after blooming, and in which settling tank conditions are maintained that do not encourage algae to swim against a current, and in which the heavier-than-water algae instead sink toward a funnel shaped harvest exit port at the bottom of the settling tank, and in which optional recirculation of clarified liquid near the top of the settling tank is provided back to the main bioreactor, with top-water clarification occurring as the algae sink to the funnel shaped bottom, and in which a concentrating effect is achieved via sedimentation of the sinking algae prior to their exit at the harvest exit port.

112. The invention further includes the system of preceding section 2, wherein a motorized roller brush cleaning assembly, a squeegee cleaning assembly, or a combination motorized-roller-brush-and-squeegee cleaning assembly is parked above the rotating auger blade assembly during a bloom cycle, and wherein during periodic cleaning cycle, the bioreactor is drained of algae suspension and filled with cleaning solution which temporarily replaces the algae pool, and in which cleaning cycle, the auger rotation direction is reversed and the rotation speed is slowed to a low rotation speed, and in which the cleaning assembly is lowered to synchronously mesh with the auger blades, wherein the auger blade rotation draws the cleaning assembly down through the turns of the auger blade, and wherein the motorized roller brushes and/or squeegee elements of the cleaning assembly clean the auger blades over the entire length of the auger, and in which the auger stops when the cleaning assembly reaches the bottom of the auger and reverses direction, drawing the cleaning assembly back to the top along a vertical guide track, and in which the cleaning assembly disengages from the auger blades at the top and is reparked above the auger blades, and in which the bioreactor is rinsed of cleaning solution and refilled with seed algae suspension in preparation for the next bloom cycle.

### BRIEF DESCRIPTION OF THE DRAWINGS

FIG. 1 is a graphical representation of our bookkeeping/trend-analysis model's projected targets for global CO₂ emissions (1), contingency capture (2), impact capture (3), and atmospheric PPM CO₂ accumulation impact (6) required to avert 450 ppm tipping points (5) and restore a preindustrial atmosphere of 280 ppm CO₂ (9) in the 21^{st} century. Curve 3 is actual impact capture of CO₂ at 10 GtC/yr. Curve 2 has an added 40% capture contingency allowance for unexpected problems, delays, and severe weather interruptions. Curve 2 (17 GtC/yr) is our recommended “fair-weather” CO₂ contingency capture target capacity (GtC/yr). Curve 2 contingency is expected to be necessary to achieve the actual capture impact of curve 3, offsetting curve 1 global emissions and meeting the accumulation targets of curve 6 in spite of problems, delays, interruptions, etc. Notice that an extra capture-time contingency allowance of 10 years is also indicated by curve 2. (Note: The PPM CO₂ accumulation impact curve 6 factors in natural sinks and land-use change emissions.)

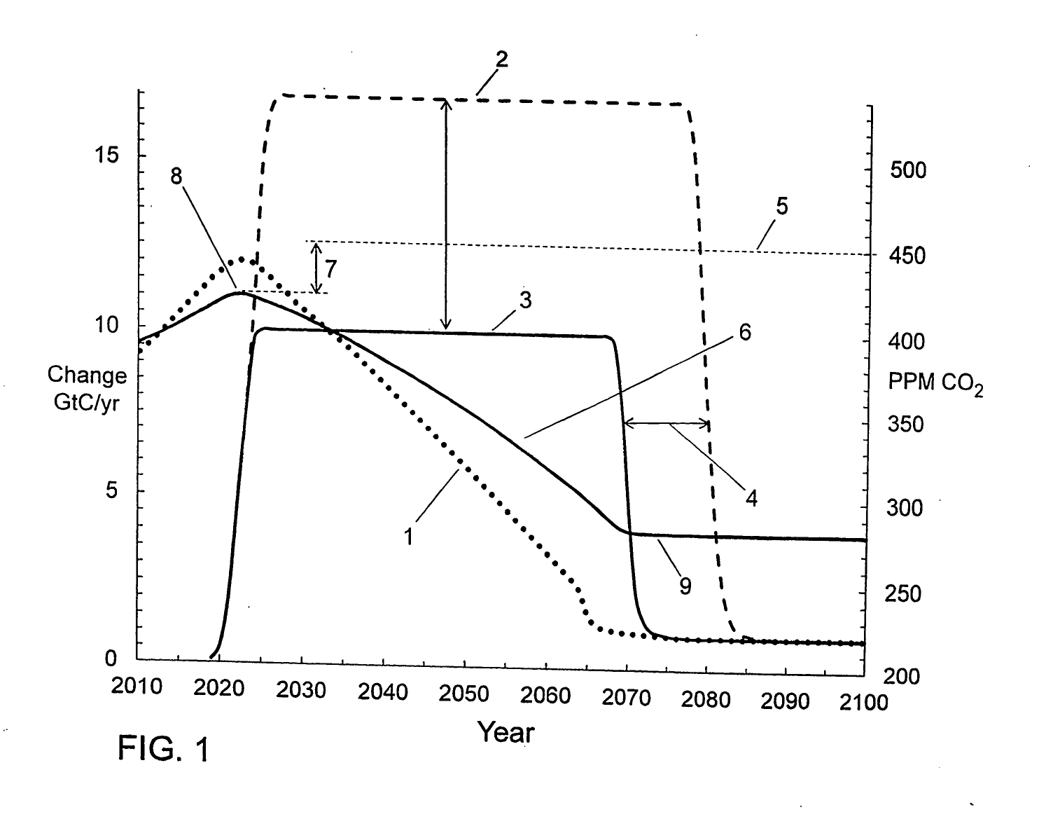

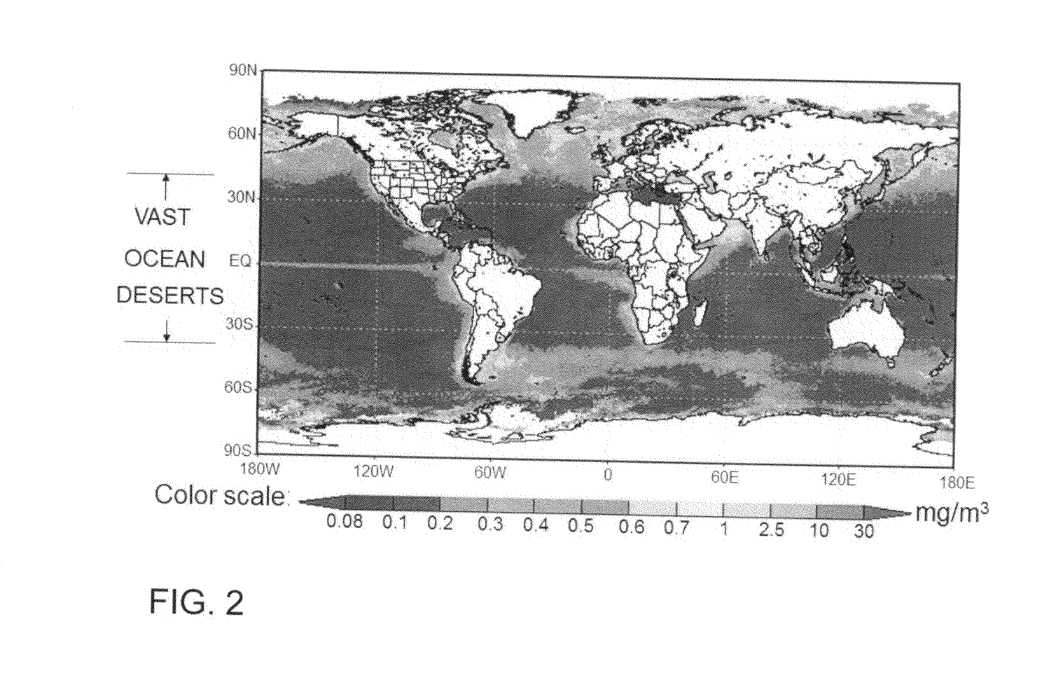

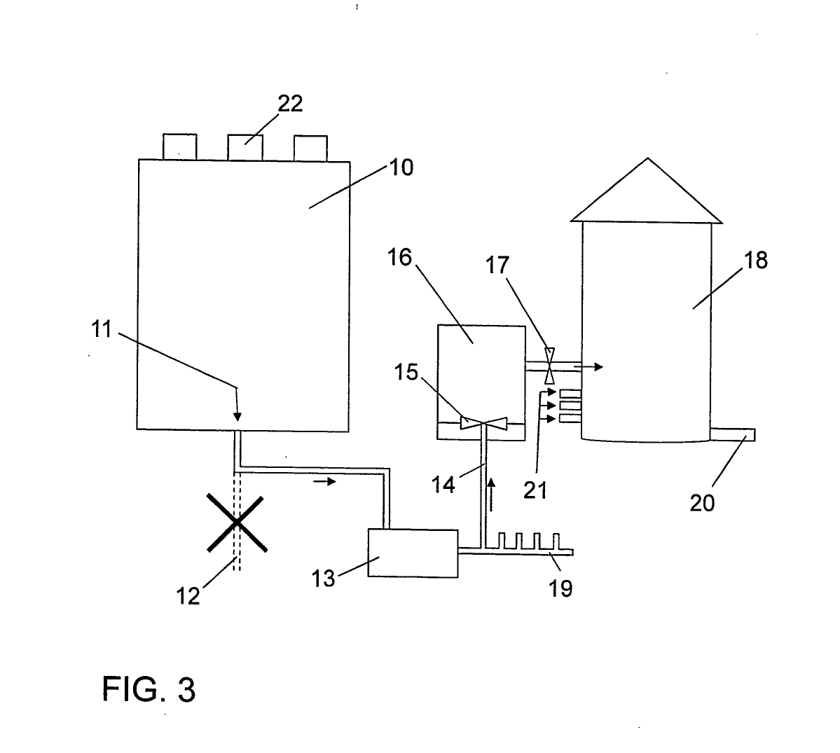

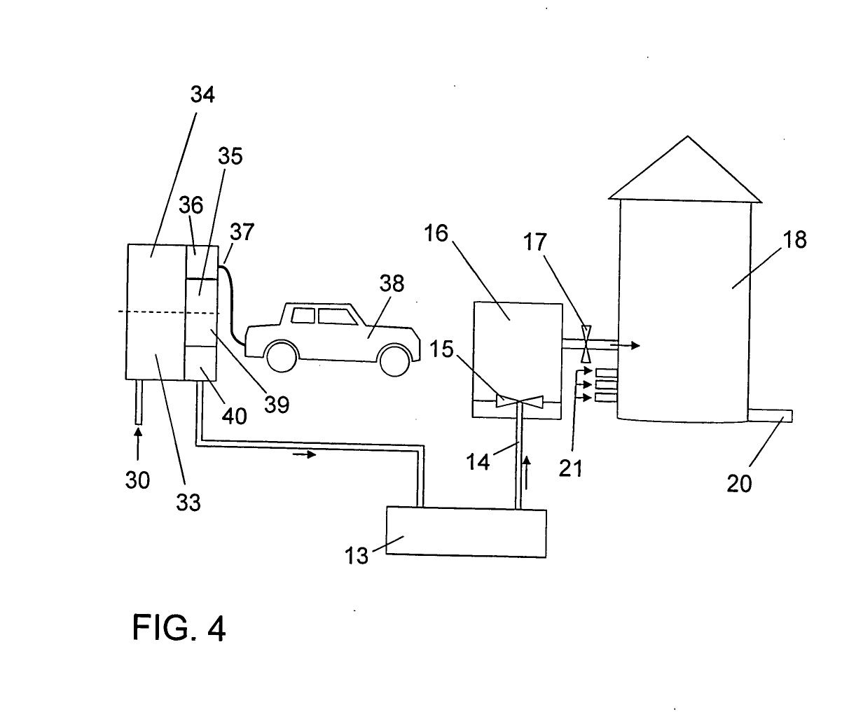

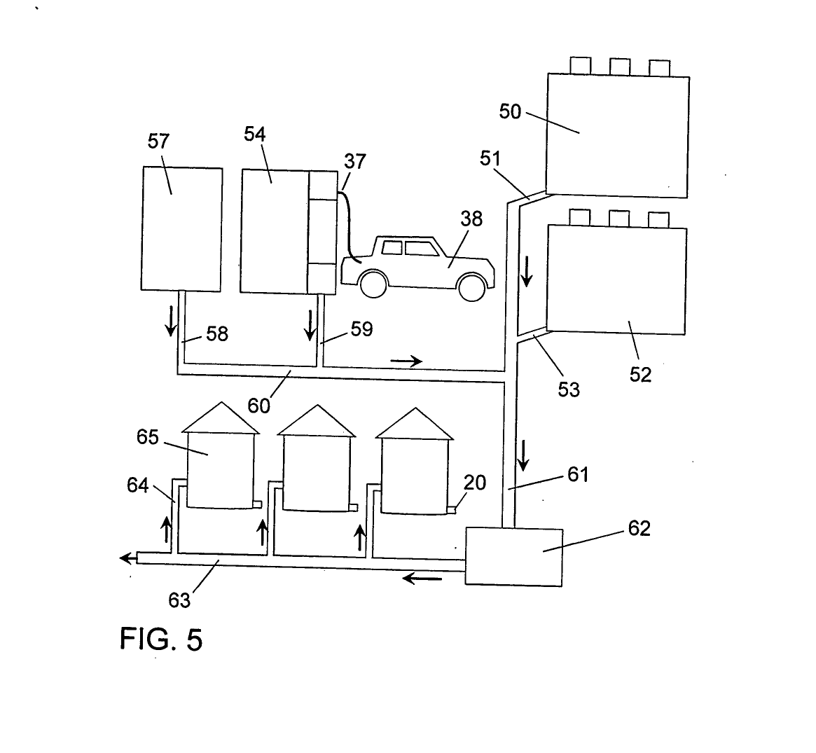

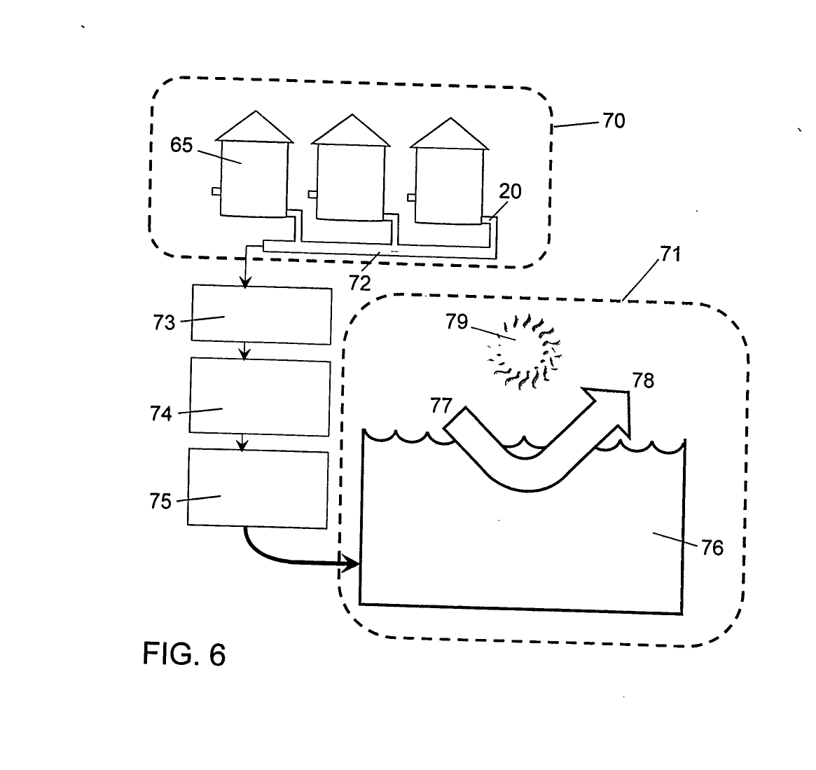

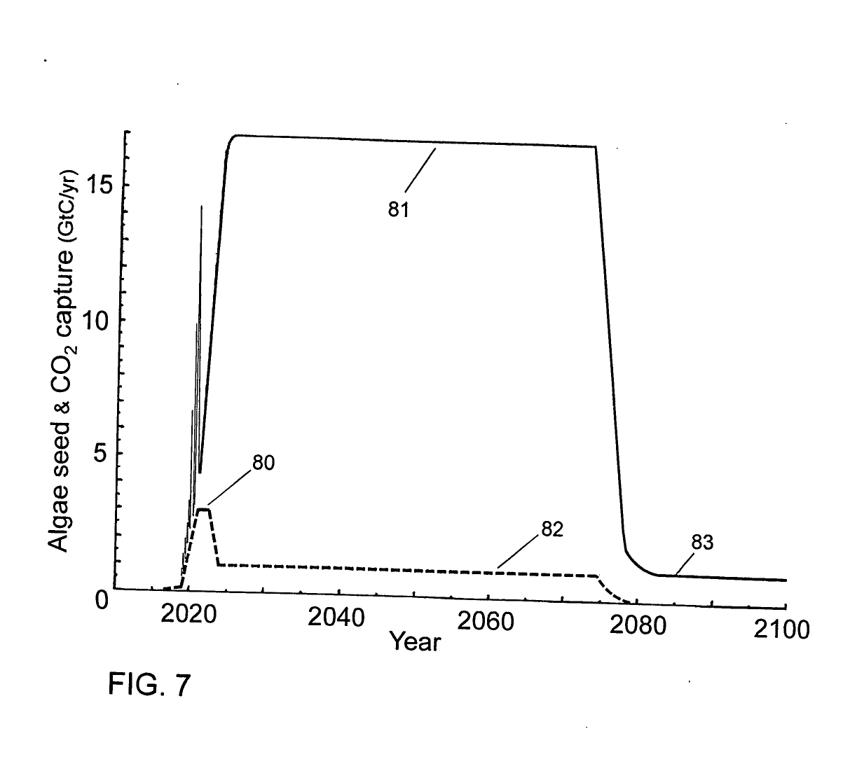

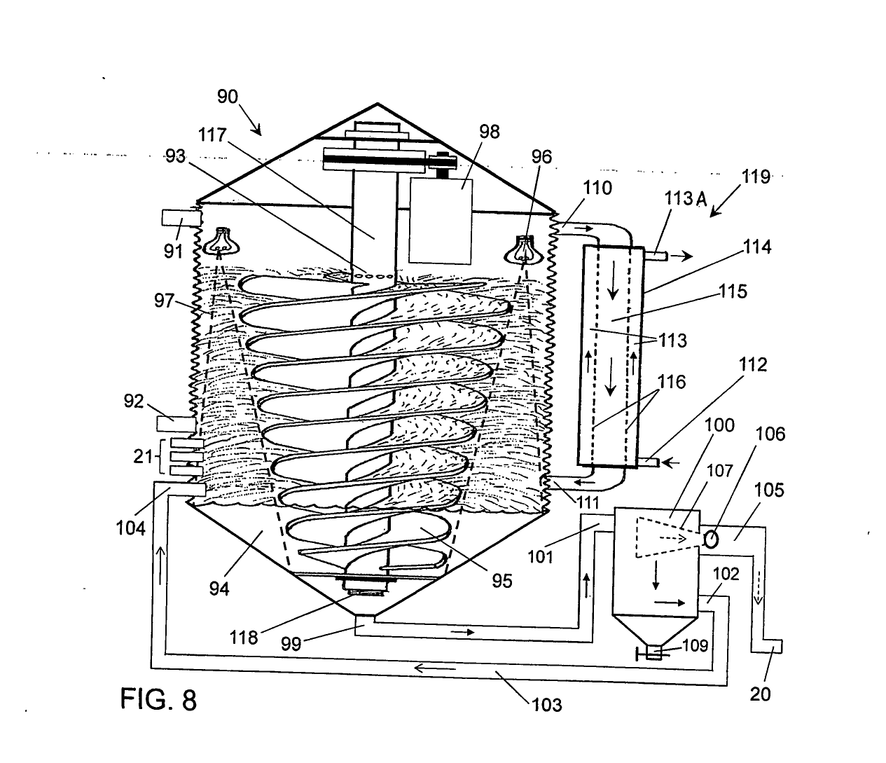

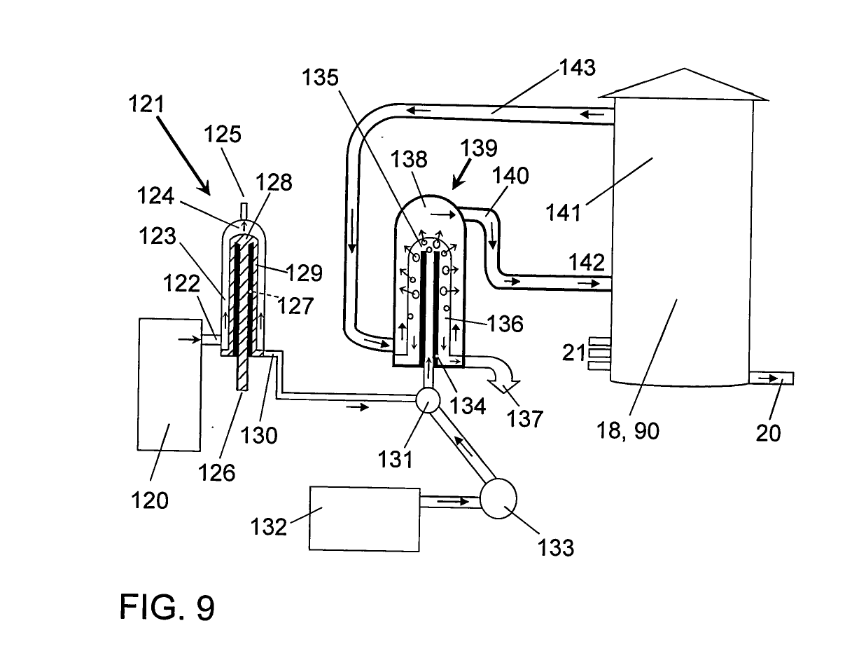

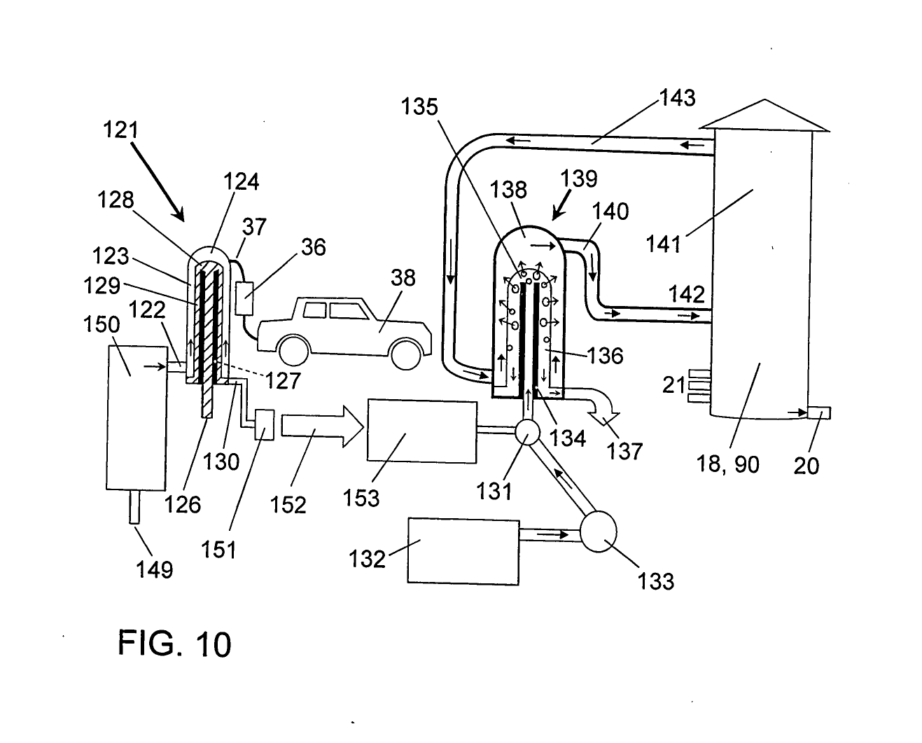

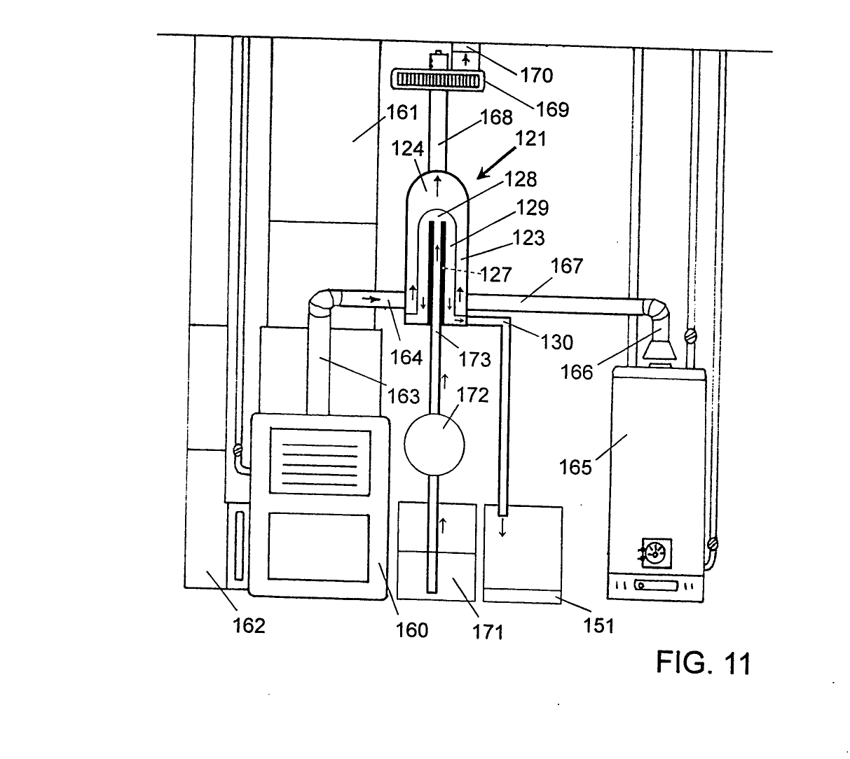

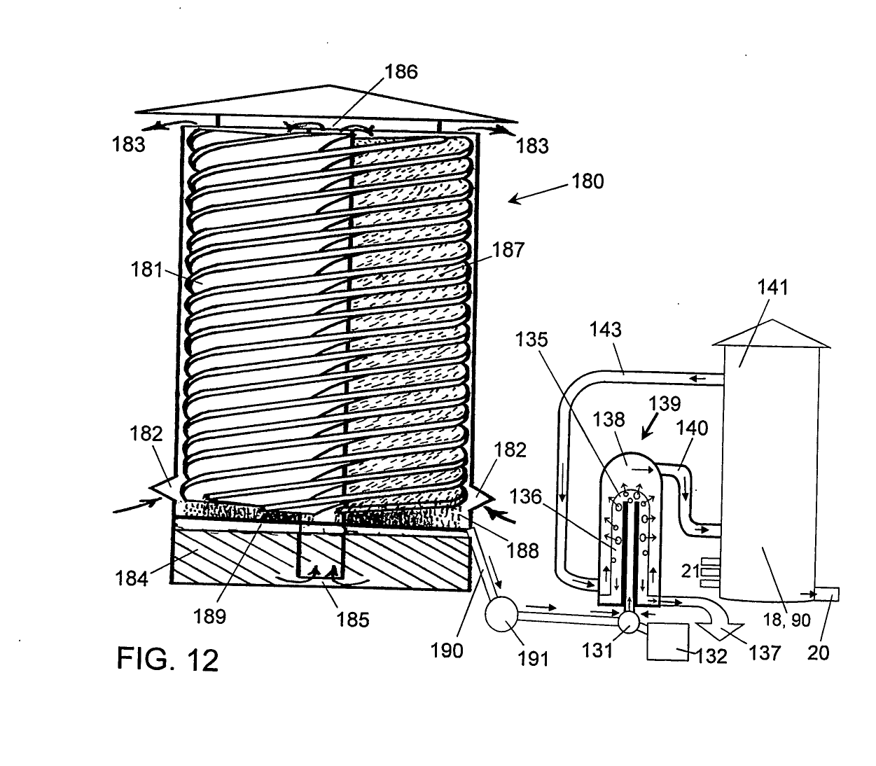

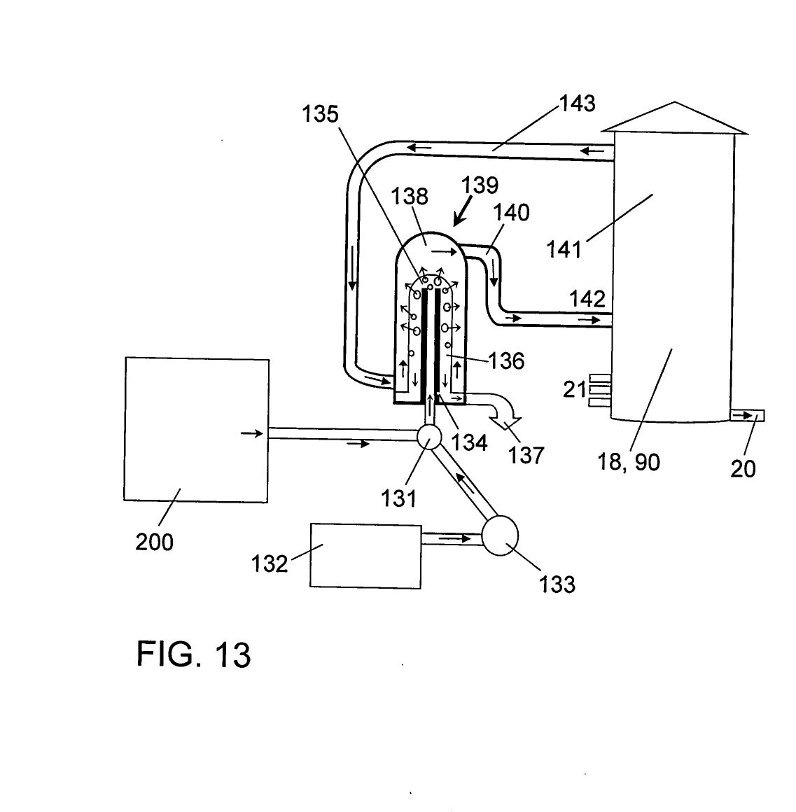

The atmospheric CO₂ accumulation impact curve (6) is computed from the difference between the CO₂ impact capture curve (3) and the CO₂ emissions curve (1), expressing the differential in ppm and applying that differential to offsetting the accumulation of previous years. FIG. 1 is a graphical illustration of the desired goal of reversing CO₂ warming by 2075 (9), plus the levels of annual emissions control (1) and invention CO₂ capture capacity (2, 3) required to achieve that goal.

Curves 1-3 have units of GtC/yr and are to be read from the “Change” axis. Reference line 5 (twin tipping points), items 8 and 9, and all points on curve 6 are to be read from the PPM CO₂ axis.

FIG. 2 is a global gray-shaded contour map of average 2008 ocean chlorophyll activity in mg/m³, from NASA satellite photos, using NASA's GIOVANNI public access plotting software with a chlorophyll—a digital filter applied. This indicates the extent of global algal blooming. The existence of vast “ocean deserts” (in which little or no algal blooming is observed for 2008) is apparent in the open seas south of Seattle, Spain, and Japan. Photic-zone nutrients are depleted and nearly no blooming occurs on average in these vast ocean deserts. Warm stratified seas south of Seattle, Spain, and Japan currently prevent the upwelling of nutrient-rich deeper waters to replenish depleted photic-zone nutrients, and an overabundance of Antarctic krill and zooplankton grazers devour what little seed algae is left.

From the vast ocean deserts indicated in this figure, it is clear that the power and full global capacity of ocean algal blooming currently lies dormant. In order to stimulate rapid, prodigious algal blooming and up to 14 GtC/yr of global CO₂ capture at sea, new triggers (other than ice-age triggers) such as this invention system provides would have to be activated.

FIG. 3 is a diagram of a Type #1 (SCF-CO₂ path) stage-1 invention configuration initially involving a prior-art CC (carbon capture) coal-fired or gas-fired electric power plant (10). FIG. 3 is a diagram of a Type #1 stage-1 invention system used as a prelude to the invention stage-2, 15× amplified ocean capture of FIGS. 6, 7. Using whole-earth carbon accounting, the two stage invention (FIGS. 3, 6) can impart a substantial (700%) negative carbon footprint to coal-fired or gas-fired CC electric power plants. The figure includes both prior-art and invention elements. Items 10, 11, 12, and 22 comprise a modern prior-art CC coal-fired or CC gas-fired electric power plant which is capable of capturing at least 50% of its carbon dioxide emissions as supercritical fluid carbon dioxide, SCF—CO₂(11). The other 50% still escapes (22) to atmosphere, but in prior-art pilot systems, the first 50% is normally pumped underground (12) into subterranean porous rock structures for storage. The FIG. 3 invention (13-20) eliminates the prior-art burial pipe (12) and redirects the SCF-CO₂ to an invention system holding tank (13). From there, SCF-CO₂ is invention-decompressed (14-16) from high supercritical fluid pressure, into an invention system medium-pressure gas holding chamber (16). From there, stage-1 of the invention system involves the medium pressure CO₂ being further decompressed (17) and injected into an invention algae conversion silo (18). The invention stage-1 silo has been pre-seeded with high-density (heavier than water) marine algae seed suspended in salt water or sea water. Nutrient and pH buffer are also provided (21). As the algae seed blooms further, injected CO₂ is consumed by photosynthesis and/or coccolithogenesis (calcification), thereby producing additional algae of the same type at the harvest output (20). An invention harvest auger (20) removes excess algae from the silo as fast as it blooms for FIG. 6 transport to seaports where FIGS. 6, 7 stage-2 invention ocean amplification begins. In stage-2 (see FIGS. 6,7), the harvest silo seed (20) may bloom another factor of 15× at sea. With nominally 50% CO₂ lost (to atmosphere) via FIG. 3 exhaust stacks (22), the overall 2-stage invention (FIGS. 3, 6, 7) amplification factor is reduced to about 8×, but this still means that, for every 1 ton of CO₂ produced in prior-art CC coal or CC gas combustion, nominally 7 more tons of CO₂ will be captured at sea. This imparts nominally a 700% net negative carbon footprint to electric power production by CC coal-fired or CC gas-fired power plants, using whole-earth carbon accounting. By this means the invention system enables CC clean-coal and CC gas-fired electric power plants to become primary engines for global atmospheric CO₂ reduction. Invention-enhanced CC clean-coal and CC gas-fired power plants will become drivers of (net) carbon sinking instead of carbon sourcing and contribute substantially to the 17 GtC/yr amplified contingency capture requirement (curve 2) of FIG. 1 and also the 10 GtC/yr impact capture requirement (curve 3) of FIG. 1.

FIG. 4 diagrams an invention system for imparting a substantial negative carbon footprint to transportation. It's the same as FIG. 3, except the concentrated CO₂ source in FIG. 4 is a prior-art natural gas (methane, CH₄) reformation system for making hydrogen (H₂) as a transportation fuel, instead of a CC gas- or CC coal-fired electric power plant. FIG. 4 essentially diagrams a second embodiment of Type #1 stage-1 invention system used as a prelude to the invention stage-2 15× amplified ocean capture of FIGS. 6, 7. Using whole-earth carbon accounting, the 2^{nd} two-stage invention embodiment (FIGS. 4, 6) is capable of imparting a substantial (1400%) negative carbon footprint to transportation.

The figure includes both prior-art and invention elements. Items 30-37 comprise a prior art methane reformation system in which natural gas (methane-30) is injected into steam (33, 34) which (in two stages) cracks off the carbon in the prior-art reformation process, leaving a 2^{nd} stage prior-art mixture of CO₂ and H₂. Separation stages (35) isolate the hydrogen for compression (36) and use as a transportation fuel (37) for hydrogen powered vehicles (38) which are illustrated as an automobile in this nonlimiting example. At this point, prior art ends. The invention begins with isolating CO₂ as a compressed gas, liquid, or super-critical fluid (SCF-CO₂, 40).

Invention stage (39) isolates CO₂ as a byproduct of methane reformation, and removes it (40) in the form of compressed CO₂ (not illustrated), liquid CO₂, or supercritical fluid (SCF-CO₂, illustrated—40) in an invention separation stage (39) into purified components H₂ (37) and CO₂ (40). The hydrogen (H₂) may be used to fuel transportation (37, 38) and the CO₂ may be compressed and/or liquefied as super critical fluid (40, SCF-CO₂). The SCF-CO₂ may be stored (13), decompressed (14-17), and converted to salt water algae (18), and continuously harvested (20) for distribution to the next stage (stage-2, operations at sea), exactly as in FIGS. 3, 6, and 7. In stage-2, (FIGS. 6, 7) the harvested silo seed may bloom another factor of 15× at sea. This means that for every 1 ton of CO₂ produced in stage-1 natural gas reformation (to make hydrogen), about 14 more tons of atmospheric CO₂ will be captured by stage-2 at sea. This imparts nominally a 1400% net negative carbon footprint to hydrogen-fueled transportation, using whole-earth carbon accounting. That's important, because hydrogen-fueled transportation would otherwise have a positive carbon footprint (from the CO₂ released by natural gas reformation to initially produce the hydrogen). The dual-stage invention will enable transportation to become a primary engine for global atmospheric CO₂ reduction. Transportation will thereby become a driver of net carbon sinking instead of carbon sourcing and contribute substantially to the 17 GtC/yr amplified fair-weather contingency capture requirement (curves 2 and 81) of FIGS. 1 and 7, as well as the 10 GtC/yr impact capture requirement (curve 3) of FIG. 1.

FIG. 5 is a diagram of stage-1 land-based invention systems including coal-fired CC power plants (50), gas-fired CC power plants (52), hydrogen production systems (54, 37) including natural gas reformation, oil gasification, and coal gasification, plus a variety of other anthropogenic, land-based CO₂ sources (57) including cement plants, kilns, blast-furnaces, refineries, factories, incinerators, crematoriums, home and building heating flues, and other sources can all converge their concentrated captured CO₂ (51, 53, 58, 59, 60, 61) into holding reservoirs and/or decompression systems (62) that supply (63, 64) arrays of algae conversion silos (65).

FIG. 6 is a diagram of how invention system stage-1 (70) couples to invention system stage-2 (71), the final ocean-amplified CO₂ capture process. In this two-stage process, fast-sinking (heavier-than-water) marine algae harvested from FIGS. 3-5, 9, 10, 12, and 13 land-based algae conversion silos (18, 65, 90) will be put into FIG. 6 stage-2 invention stasis-supporting cargo containers which will be transported to seaports (73), where they'll be loaded onto cargo ships for distributing to floating seed repositories (74) on the open seas. From there, the invention stasis-supporting cargo containers will be transferred to seed boats (75) which fan out to seed 1-3 GtC/yr of algae+nutrient into 70% of Earth's ocean surfaces (76) under exceptional invention system conditions which favor prodigious ocean blooming (and corresponding capture of carbon dioxide (77) from the atmosphere) to the light penetration (opacity) limit within approximately two weeks. This is selective invention-induced stage-2 ocean blooming (71) which is dominated by the invention high-density fast-sinking algae seeded from the invention stasis-supporting cargo containers (73) filled from invention land-based invention stage-1 algae silos (65). Stage-2 ocean starter seed levels (75) will be so high (3 GtC/yr at first, with frequent reseeding) that ocean grazers will only consume a maximum of ⅔ of the invention-produced starter seed (2 GtC/yr estimated global grazer appetites) before it has a chance to bloom. At least ⅓ of the starter seed (˜1 GtC/yr) will remain un-eaten and will be available to seed stage-2 amplified ocean blooming to the opacity limit within two weeks. At this point the invention-supplied nutrient doses are calculated to run out, and the algae bloom will die and rapidly sink (owing to its heavy calcium carbonate exoskeleton). The fast-sinking property will enable the dead algae bloom to clear the photic zone by the end of each month. This key invention-enabled feature will prepare the photic zone for reseeding at the beginning of the next month and it will uniquely enable twelve large blooms per year, instead of just one. By this means, stage-2 invention-amplified ocean algal blooming (71) can capture up to 14 GtC/yr of carbon dioxide which combines with the stage-1 invention land capture rate of up to 3 GtC/yr to create the 17 GtC/yr total invention-enabled carbon capture capacity required earlier by curve 2 of FIG. 1. At the end of each bloom cycle in FIG. 6, stage-2 invention aerator boats may fan out from the seed repositories, to bubble compressed air or oxygen to within 5 meters of the sea floor in shallow coastal waters. This will prevent post-bloom anoxia from secondary bacterial blooming in coastal waters. In the open seas, rapid sinking should carry the dead algae quickly to the deep sea floor, where frigid water temperatures (between zero and 4 degrees C.) will likely preserve them until they get buried by sedimentation at the rate of about 1 mm/year of marine “snow”. This should prevent post-bloom anoxia from developing.

FIG. 7 is a graphical projection of results expected from two-stage invention system amplification. It is a graph of anticipated invention seed & capture rates in GtC/yr (giga-tons carbon per year, or billion tons carbon per year, as CO₂ (carbon measure)) versus time. Dashed curve (80, 82) is the anticipated ocean seeding rate, in terms of high-density, fast-sinking starter algae seed, which the invention system will selectively enable. This is nominally 1 GtC/yr (82) from 2023-2075. A front end seeding “bump” (80) of nominally 3 GtC/yr is recommended from 2020-2023, in order to offset ocean grazer feeding appetites. Grazers are anticipated to (globally) eat approximately 2 GtC/yr of the seed, before it has a chance to bloom. Seeding 3 GtC/yr (80) will satiate grazer appetites and leave 1 GtC/yr uneaten to serve as the net amount of available starter seed. By 2023, sufficient ocean blooming is anticipated to allow seed levels to diminish to 1 GtC/yr (82), and by then grazers should be feeding from the amplified bloom (81), rather than the starter seed. (Note: At that time, the extra 2 GtC/yr of available land-harvested seed production capacity may be diverted to other algae applications such as silage, animal feed, fish farming feed, fertilizer, biofuels, and/or inland lake/river revitalization (algal cleansing of agricultural runoff).) Such high level ocean seeding will be invention-system enabled by the land-based algae bioreactors which produce up to 3 GtC/yr of seed from concentrated land-sources.

Curve (81) is the anticipated stage-2 15×-amplified ocean CO₂ capture response enabled by 1 GtC/yr invention ocean seeding (82). Essentially, 14 GtC/yr of amplified natural ocean capture (CO₂) is expected from 1 GtC/yr of invention seeding. Additional accounting for anticipated land-based capture of 3 GtC/yr raises the curve (81) total land-and-sea capture rate to 17 GtC/yr, as required earlier by FIG. 1. This represents the awakening of nature's “green giant” with oceans doing the “heavy lifting” (81) in response to a relatively small invention-enabled seed level (82). A series of sharp spikes on the rising edge of the capture curve (81) represents anticipated transient fluctuations in the amplified capture rate as overpopulated zooplankton grazers devour invention starter seed early in the seed program, and as decimated populations of predators return (re-proliferate) to eat the grazers. As grazer and predator population ratios fluctuate in response to the seeding curve, a series of spikes are expected until the natural balance of grazer and predator is finally restored. (The situation is currently unbalanced with over-populated grazers (copepods, krill, etc.), due to commercial overfishing of their predators.) Once natural balance has been restored (reproliferating decimated and endangered species of marine life and restoring their numbers to burgeoning populations last seen in the 18^{th} and mid-19^{th} centuries), then the capture curve (81) can finally rise to its 17 GtC/yr (land and sea) maximum and be sustained at that level, as long as the seeding program (82) continues. Restoration of marine life populations to mid-19^{th} century levels (or earlier) will be a significant side-benefit of this invention.

FIG. 8 is a diagram of internal workings of FIGS. 3-6, 9, 10, 12, and 13 invention stage-1 algae conversion silo (18, 65, 90). Concentrated CO₂ enters the silo headspace at inlet 91 or 92 of FIG. 8. For the invention Type #2 (NaOH starter path) and Type #3 (NaHCO₃ starter path) stage-1 embodiments of FIGS. 9-13, port (91) of FIG. 8 is a recirculation port from which headspace (8) gases are withdrawn (out-flow) by fan (not shown), cycled through the gas-liquid separators (139) of FIGS. 9-13 where they pick up released CO₂ (138) and then the gases (with added CO₂) are returned to the silo headspace at port (92). The algae silo headspace is thus “common” to the gas-liquid separator headspaces (138) of FIGS. 9-13.

Referring to FIG. 8, the lower extent of rotating auger (95) is immersed in a high-density marine algae suspension (94) which is continuously lifted from the suspension pool (94) by auger (95) which (at 50 rpm in a non-limiting example) slings suspended algae off the edges of the auger blade in thin watery helical fountain sheets throughout most of the silo. Illuminators (96) shining down through the thin helical fountain sheets expose algae to light energy for driving photosynthesis. Light-activated algae seed blooms on exposure to headspace CO₂ which is consumed in the blooming process. The activated helical fountain sheets fall back into pool 94, either falling directly or running down the sides of the silo. Auger 95 then recirculates the suspended algae back through the helical fountain, over and over again, enabling repeated exposure to headspace CO₂. The resulting algal blooming is continuous, occurring at an exceptionally high rate. A smaller auger (not shown) transfers algae out of pool 94 via port 99 as fast as it blooms and injects (101) it into an adjacent separation tank (100).

The separation tank (100) is relatively large diameter to cause a significant reduction in flow velocity at the same flow rate as 101. This velocity reduction is important, because it suddenly offers the tiny algae (e.g. 2 μm in diameter and having flagella for motility in a nonlimiting E. huxleyi example) an opportunity to swim against the current, if they so desire. What is needed next is a reason for the algae to swim against the current so that they will concentrate in the upper end of the separation tank. That impetus is provided by tank (100) and its main downward flow path being dark and essentially devoid of both CO₂ and nutrient, whereas an attractant light beam (beacon 106, 107) is positioned within the mouth of a harvest exit tee (105) located near the upper extent of tank (100). With the main separation tank volume (100) and path (101→102) being essentially devoid of light, and with the flow velocity significantly reduced at large tank diameter, the algae may swim against downward current (101→102)—swimming upward instead toward the attractant beacon (107) and illuminator globe (106) supplied at the mouth of the harvest exit tee (105). The exit tee and harvest exit path (105→20) are smaller in diameter again and, even though the exit path (105→20) flow rate is low, this diameter reduction raises flow velocity (relative to path 101→102) enough that any algae which appear at the mouth of the exit tee (106, 105) will be sucked into harvest exit flow path (105). Marine algae may be continuously harvested as ocean seed at the harvest output of the silo (20). The bioreactor is continuous, self-concentrating, and will promote prodigious algal blooming at output (20). About 85% of the algal bloom will continuously exit via the harvest path (15) in a nonlimiting example, with about 15% recirculating via path (102-104). Any dead algae will sink and may be periodically removed at (109).

A pH buffer (e.g., phosphate buffer, in a nonlimiting example) added (21) to the algae pool (94), buffers the pool against acidification (carbonation) from high level headspace CO₂. Buffering the pH at nominally 8.32 will maximize coccolithophore algae blooming and prevent softening or acidic dissolution of the coccolithophore exoskeleton (CaCO₃). As algae is continuously harvested (20) as a concentrated suspension, replenishment sea water or salt water, nutrient, and pH buffer are provided at the replenishment inputs (21) to the silo algae pool (94).

Oxygen produced during photosynthesis is continuously removed by an oxygen removal system (119, 110-116) based on at least one oxygen-permeable membrane (116), which is tubular in the nonlimiting FIG. 8 embodiment, and a far-side exhaust sweep gas (113), such as nitrogen (112) in a non-limiting example. A tubular membrane (116) and far-side annular sweep gas space (113) are depicted in this non-limiting example. Only one oxygen removal system (119) is depicted, but multiple units (of 119) mounted on the same silo would also be within the scope of the invention. In this oxygen removal system (119), a fraction of the silo headspace gas would be drawn by fan (not shown) into the removal system at 110 and down through the removal system center (115).

Oxygen in the mixture would selectively permeate membranes (116) into a nitrogen sweep gas (113) introduced at 112. The nitrogen sweep gas (113) would remove all of the permeating oxygen and exhaust it at 113A. CO₂ in the mixture would continue down the center (115) and wouldn't permeate the tubular membrane. It would simply rejoin the silo headspace at 111, just above pool 94.

This stage-1 invention bioreactor system (90) may be considered a pseudo-anaerobic bioreactor since oxygen is removed (119) as fast as it is produced by photosynthesis. Algal blooming will therefore proceed under pseudo-anaerobic conditions which will enhance bloom rates, because oxygen otherwise acts as a photosynthetic inhibitor (above a certain point), and its continuous removal (119) will accelerate blooming.

FIG. 9 is a diagram of Type #2 (NaOH starter path) of a stage-1 invention configuration involving land-based invention continuous algal conversion of CO₂ from a generic (either invention or prior art) CO₂-laden gas mixture source (120) to high density marine algae as a prelude to the stage-2 15× invention-amplified ocean capture of FIG. 6. In FIG. 9, Type #2 includes a CO₂-laden gas mixture (10), lye capture path (122-130) with a thin film reactor (121, lye scrubber), sodium bicarbonate as a capture product (130), acidification (131-133), re-release of CO₂ from a bubbling film of salt water (136) as it overflows (135) a standpipe (134) within a gas-liquid separator (139) in which released CO₂ in the separator headspace (135) is swept away to inject a high-efficiency, high-capacity stage-1 bioreactor (algae conversion silo (18, 90)) with elevated CO₂ levels. Algae harvested at stage-1 bioreactor output (20) may then seed the stage-2 15× amplified ocean capture of additional CO₂ in FIGS. 6, 7.

FIG. 10 is a diagram of a Type #2 invention system for imparting a substantial negative carbon footprint to transportation. It is the same as FIG. 9, except that the CO₂-laden gas mixture source in FIG. 10 is a prior-art natural gas (methane, CH₄) reformation system for making hydrogen (H₂) as a transportation fuel. FIG. 10 is a diagram of a Type #2 stage-1 invention system to be used as a prelude to the invention stage-2 15× amplified ocean capture of FIG. 6. Using whole-earth carbon accounting, the two-stage invention (FIGS. 10, 6) is capable of imparting a substantial (1400%) negative carbon footprint to transportation, with refueling at a home hydrogen production station or a public hydrogen filling station, both of which employ lye capture of reformation process CO₂. FIG. 10 includes both prior-art and invention elements. Item 150 comprises a prior art methane reformation system in which methane (149) is injected into steam (150) which cracks off the carbon in a two-stage prior-art reformation process, leaving a final mixture of CO₂ and H₂. At this point (122), prior art ends and the invention begins with a thin film lye reactor (121, 122-130) for isolating hydrogen (H₂) (37) and compressing it (36) for use as an ultra-clean transportation fuel for hydrogen-powered vehicles (38). A hydrogen powered car is depicted, but that could equally be a van, truck, bus, train, boat, or even an aircraft. FIG. 10 isolates CO₂ as a sodium bicarbonate (NaHCO₃) drain solution (130) collecting in a local pickup vessel (151). The pickup vessel (151) may be periodically transported (52) and emptied into a regional or district NaHCO₃ receiving station (153) where the NaHCO₃ is acidified (131-133) to re-release CO₂ from a bubbling film of salt water (136) as it overflows (135) a standpipe (134) within a gas-liquid separator (139) in which released CO₂ in the separator headspace (138) is swept away for injection into an adjacent high-efficiency, high-capacity stage-1 bioreactor (algae conversion silo (18, 90)) with elevated CO₂ levels with the CO₂ being converted to marine algae, and continuously harvested (20) for distribution to the next stage (stage-2, operations at sea), exactly as in FIGS. 9 and 6. In stage-2, (FIGS. 6, 7) the harvest silo seed may bloom another factor of 15× at sea. This means that for every 1 ton of CO₂ produced in FIG. 10 stage-1 natural gas reformation (to make hydrogen), about 14 more tons of CO₂ will be captured by stage-2 at sea. This imparts nominally a 1400% net negative carbon footprint to hydrogen-fueled transportation, using whole-earth carbon accounting. That's significant, because hydrogen-fueled transportation would otherwise have a positive carbon footprint (from the CO₂ released by natural gas reformation to initially produce the hydrogen). The two-stage invention system will enable transportation to become a primary engine for global atmospheric CO₂ reduction. Transportation will thereby become a driver of net carbon sinking instead of carbon sourcing and contribute substantially to the 17 GtC/yr amplified contingency capture requirement (curves 2, 81) of FIGS. 1, 7, as well as the 10 GtC/yr impact capture requirement (curve 3, FIG. 1).

FIG. 11 diagrams another Type #2 stage-1 lye capture path invention embodiment involving a lye scrubber for home and building flues. Hot exhaust flue gases (163, 166) may optionally be cooled by adding auxiliary cooling air (not shown) prior to tangentially entering (164, 167) a thin film reactor (121) which functions as a lye scrubber. Lye solution (171, 173) is pumped (172) to overflow (128) a standpipe (127) within the reactor (121) so it flows continuously down the outside of the standpipe as a thin film of lye (129) which readily absorbs CO₂ from a rising vortex counter-flow (123) of flue gases encircling the standpipe in the annular space (123) of the reactor (121). The lye film (129) is thereby converted to sodium bicarbonate (NaHCO₃) solution before it reaches the bottom of the reactor and exits via the NaHCO₃ solution drain (130) to collect in pickup vessel 151. Upon filling, this vessel may be transported (152) to the district NaHCO₃ receiving station (153) of FIG. 10 for subsequent algae conversion (20) and FIGS. 6, 7 stage-2 invention amplified ocean capture of 15× more CO₂ than the original FIG. 11 home and building flues produced. By this means home and building furnaces (160), water heaters (165), etc. may gain a 1400% net negative carbon footprint (whole-earth carbon accounting) and contribute substantially to the 17 GtC/yr amplified contingency capture requirement (curves 2, 81) of FIGS. 1, 7 as well as the 10 GtC/yr impact capture requirement (curve 3, FIG. 1). Crematorium and incinerators (not shown) may also use a FIGS. 10, 11 lye scrubber for CO₂ capture, and transport (152) of the NaHCO₃ pickup vessel (151) to the district NaHCO₃ receiving station (153) of FIG. 10, stage-1 algae conversion (18, 90), and FIGS. 6, 7 stage-2 15× amplified ocean capture of additional CO₂.

FIG. 12 diagrams an outdoor air embodiment for Type #2 stage-1 CO₂ capture with a large lye (NaOH) fountain bin (180) producing NaHCO₃ (190) as the capture product. This is once again a prelude to the 15× invention-amplified stage-2 ocean capture of FIGS. 6, 7. In FIG. 12 (Type #2 stage-1 invention Outdoor Air Embodiment) and algae conversion silo (18, 90). The lye fountain bin (180) houses a large, slow-rotating (e.g., 9 rpm in a non-limiting example) air auger (181) which produces a CO₂-laden air-draw at base perimeter inlets (182) and pushes stripped air out via exits (183). The air auger has a hollow drive shaft with its lower extent (185) protruding through a sealed false bottom (189) and immersing in a lye solution (sodium hydroxide, NaOH solution reservoir (184)). The hollow auger driveshaft houses a smaller, higher speed auger (not shown) which uptakes lye (185) and lifts it up through the hollow main air auger shaft, spilling lye out at an outflow (186) at the top of the air auger, spilling over the air auger blades, wetting them and causing a falling film of lye (187) to run continuously, spiraling down the large auger blades. The downward flowing lye film absorbs (scrubs) CO₂ from the rising air column and the resulting NaHCO₃ capture solution spills off the bottom (188) of the auger blades onto the sloping false bottom (189) where it enters the NaHCO₃ drain (190) and proceeds to acidification (131, 132) for re-releasing its CO₂ (138) with subsequent injection (140) into the algae conversion silo (18, 90) as before, for conversion to marine algae for seeding stage-2 ocean amplified capture (FIGS. 6, 7).

FIG. 13 is a diagram of Type #3 (NaHCO₃ starter path) of a stage-1 invention configuration involving land-based invention continuous algal conversion of carbonate or bicarbonate solution from a generic (either invention or prior art) source (200) of bicarbonate or carbonate solution (or a mixture of bicarbonate and carbonate) to high density marine algae as a prelude to the stage-2 15× invention-amplified ocean capture of FIG. 6. FIG. 13 is the same as the 2^{nd} and 3^{rd} sections of FIG. 12 beginning with acidification (131-133) of the NaHCO₃ solution to re-release CO₂ from a bubbling film of salt water as it overflows a standpipe within a gas-liquid separator in which released CO₂ in the separator headspace is swept away to inject an adjacent high-efficiency, high-capacity stage-1 bioreactor (algae conversion silo) with elevated CO₂ levels, to photosynthetically produce an algae harvest output, as before. Algae harvested at the stage-1 bioreactor output (20) may then seed the stage-2 15× amplified ocean capture of additional CO₂ in FIG. 6.

### DETAILED DESCRIPTION OF THE INVENTION

This is a multi-stage invention system comprising a multiplicity of individual stage-1 inventions or an initial prior-art concentrated carbon dioxide source combined with at least one of the individual stage-1 land-based invention capture and algae conversion systems and stage-2 invention process-enhanced ocean-amplified capture, in which all stages (and the FIGS. 3-13 multiple embodiments) comprise multiple, globally-distributed copies of the invention systems to collectively achieve a capture capacity of 17 GtC/yr, accumulating to ˜0.45 tera-ton ((carbon measure) or ˜1.65 tera-tons actual CO₂) of total CO₂ capture and safe storage from 2027-2072, restoring atmospheric CO₂ to its pre-industrial level (280 ppm) by 2075. The multi-stage system is presented here in a single patent specification in order to demonStrate how a total capture and storage capacity of 17 GtC/yr (contingency) or 10 GtC/yr (impact) may be collectively achieved by a combination of multiple invention systems to gradually reverse that portion of global warming which is attributable to CO₂. Multiple individual inventions within the multi-stage system are described in individual claims, which are in addition to the multi-stage combination systems and process claims.

Note: In order for multiple, globally-distributed copies of the multi-stage CO₂ capture and storage system to restore the atmosphere to 280 ppm CO₂ by 2075, global emissions need to be capped at 12 GtC/yr by 2023 (FIG. 1, curve 1) and gradually reduced to 6 GtC/yr by 2050, 3 GtC/yr by 2062, and 1 GtC/yr by 2078, in addition to multi-stage system contingency capture of 17 GtC/yr CO₂ and 10 GtC/yr impact capture continuously (FIG. 1, curves 2, 3) each year from 2027-2072, and permanent safe storage of the accumulated capture form (˜0.45 tera-tons, carbon measure which is ˜1.65 tera-tons CO₂—converted to marine algae which gets eaten and/or sinks to the bottom of the ocean and gets buried by ocean sedimentation). This global emissions cap and reduction schedule will be achieved, in part, from more diligent and widespread application of certain prior-art technologies and practices such as clean-coal (CC) and nuclear energy, with smaller contributions from wind and solar energy, energy efficiency and conservation, and in part from re-forestation and sweeping changes in agriculture (especially 3^{rd} world agriculture), agricultural product usage, and the western diet, transportation (e.g. fuel efficient and/or electric cars), travel (increased teleconferencing and reduced business air travel), and commuting practices (living closer to work, increased carpooling, and greater use of mass transit). Items listed in the preceding sentence are all prior-art, with more diligent and widespread application required to contribute substantially to the FIG. 1 global emissions cap and reduction schedule (1). FIG. 1, curve 1 targets will also be achieved, in substantial part, by converting a major fraction of transportation to hydrogen (H₂) fueling by about 2050. Hydrogen-powered vehicles already exist in prior-art, such as the Honda FCX-C₁₋arity (a fuel-cell car operating on hydrogen). What doesn't exist in prior art is a significant source of hydrogen fuel (or means of making it), enough to fuel a substantial fraction of all transportation by 2050 without releasing CO₂ in hydrogen production. Prior-art solar energy systems may be used to generate hydrogen by electrolyzing water, but solar energy is only viable where abundant sunshine exists and that excludes most of the industrial world. Prior-art natural-gas (methane) reformation is the primary means of today's hydrogen production, but methane reformation releases CO₂ as a major prior-art byproduct.

In our multi-stage invention, the concentrated CO₂ byproduct of hydrogen production by natural-gas reformation, oil gasification, and/or coal gasification will be converted to high density marine algae in stage-1 invention silos (FIGS. 4 and 10) and that will seed the stage-2 invention system ocean capture and storage (FIGS. 6, 7) of much larger (15× amplified) amounts of atmospheric CO₂—as that is also consumed by prodigious ocean algal blooming stimulated by the invention systems. Hydrogen production which is upstream enabled by invention systems (FIGS. 4, 10, and 6) will therefore serve double duty in 1.) contributing significantly to amplified multi-stage CO₂ ocean capture and storage (FIGS. 1, 7, curves 2, 3, 81), plus 2.) reducing CO₂ emissions on the above-listed (and FIG. 1, curve 1) schedule (as invention system enabled hydrogen production makes it possible for hydrogen to replace fossil-fuel burning in transportation).

Note: In some embodiments, portions of the multi-stage invention system may be borrowed from prior-art and then incorporated into a new larger invention system. Prior-art items are not separately claimed, and invention claims only involve them as components of a larger invention system and/or of a globally-distributed multi-stage invention combination system, which larger invention system and/or multi-stage combination system is (at once) novel, non-obvious, and desperately needed for avoiding impending near term 450 ppm CO₂ tipping points, for restoring 280 ppm CO₂ by 2075, setting the stage for subsequent global warming reversal and the elimination of ocean acidification. In addition, some portions of the larger invention and/or the multi-stage combination involve device claims and other portions involve process claims. This mixture of device and process claims is required in a single patent application in order to present the case and demonstrate the potential for an overall 17 GtC/yr CO₂ contingency capture (FIG. 1, curve 2) and 10 GtC/yr impact capture (FIG. 1, curve 3), which are both required to offset global emissions anticipated to reach 12 GtC/yr by 2023 (FIG. 1, curve 1), thereby enabling the stage to be set for gradual reversal (via FIG. 1, curve 6) of global warming.

Stage-1 is land-based capture of 1-3 GtC/yr CO₂ (FIGS. 3-5 and 9-13) and its conversion to high density marine algae. If global warming is to be reversed before 450 Ppm CO₂ tipping points are reached, it must be recognized that it won't be possible to capture 1-3 GtC/yr of CO₂ by any single means. And yet 3 GtC/yr is the initial capture rate required to effectively begin the meeting the targets of FIG. 1. The multi-stage invention therefore encompasses a multiplicity of CO₂ initial capture systems in stage-1, including both prior-art and invention stage-1 initial capture systems (FIGS. 3-5 and 9-13), in which captured and concentrated CO₂ from the multiplicity of CO₂ stage-1 initial capture systems is combined (e.g., as in FIG. 5), and the combined total of captured, concentrated CO₂ adds up to the required 3 GtC/yr initial land-based capture to seed the stage-2 ocean amplification that will be required to enable warming reversal. We estimate that 3 GtC/yr also represents the maximum stage-1 CO₂ (land-based) capture which realistically can be mustered from combined global sources and globally scaled and deployed invention CO₂ capture and algal conversion systems prior to amplification.

These multi-stage invention systems relate to global climate change, ocean acidification geo-engineering, and more specifically to global climate restoration, ocean revitalization, and fueling ultra-clean transportation with hydrogen (H₂). Climate restoration would be achieved by capturing the greenhouse gas carbon dioxide (CO₂) from Earth's atmosphere significantly faster than it is produced, and doing that over an extended period, e.g. from 2027-2075. The recommended collective capture rate by globally distributed copies of our multi-stage invention is 17 GtC of CO₂ per year contingency (FIGS. 1, 7, curves 2, 81) and 10 GtC/yr impact (FIG. 1, curve 3) each year from 2027-2072, in order to reduce Earth's atmospheric accumulation (FIG. 1, curve 6) of CO₂ to the ideal (pre-industrial) level of 280 ppm (parts-per-million) (9) by 2075.

(Note: The system 17 GtC/yr contingency capture target (curve 2), 10 GtC/yr impact 0.5 target (curve 3) and accumulation impact curve (6) assume global CO₂ emissions would be capped at 12 GtC/yr by 2023 and then reduced to 6 GtC/yr by 2050, 3 GtC/yr by 2062, and 1 GtC/yr by 2075 according to emissions curve 1.)

Total multi-stage capture of CO₂ for the period 2027-2072 would amount to approximately 0.45 tera-tons (450 billion tons, carbon measure), which is 1.65 tera-tons (actual CO₂ measure), and permanent safe storage for that much captured CO₂ is a further requirement for safely reducing Earth's atmospheric accumulation to 280 ppm CO₂ in the 21^{st} century.

The multi-stage invention systems relate more specifically yet to selectively amplified ocean algal blooming for large scale (14 GtC/yr) photosynthetic and/or coccolithophore calcification capture of CO₂ by accelerated ocean algal blooming (FIGS. 6, 7), and they relate even more specifically yet to circumventing barriers which otherwise block prior-art systems from successful global acceleration of ocean algal blooming and would prevent ocean capture of more than 1-4 GtC/yr of CO₂. Only the oceans are large enough and powerful enough to capture CO₂ on a scale matching or exceeding current and 2023-projected CO₂ emissions rates (10 GtC/yr and 12 GtC/yr, respectively). It is clear that having ocean algal blooming stalled-out at only 1-4 GtC/yr (or less) won't be a satisfactory capture rate. If climate stabilization and ocean revitalization are to be successful, capture must substantially exceed emissions. There remains a need for circumventing the existing barriers to accelerated ocean algal blooming, thereby allowing stage-2 ocean blooming to capture approximately 14 GtC/yr of CO₂ in addition to stage-1 initial land-based capture of 3 GtC/yr of CO₂, such that the total (land and sea) capture rate can reach 17 GtC/yr of CO₂ (fair-weather contingency basis) or 10 GtC/yr (average global impact basis).

Turning now to the drawings, FIG. 3 illustrates a Type #1 invention stage-1, based on an initial supercritical fluid carbon dioxide (SCF-CO₂) capture path and continuous, land-based algae silo conversion of captured SCF-CO₂ to high density, fast sinking marine algae as a prelude to FIG. 6 invention stage-2 15× amplified ocean capture of 1400% more CO₂ (at sea) than was originally input to stage-1. In a preferred embodiment of the FIG. 3 Type #1 invention stage-1 system relating to prior-art clean-coal-fired (CC, or carbon capture) electric power-plants (10) that already capture a significant or majority fraction of their CO₂ emissions as super-critical-fluid (SCF) carbon dioxide (SCF-CO₂, 11) which is piped underground (12) into porous rock structures for storage. The FIG. 1 Type #1, stage-1 system also relates to prior-art CC gas-fired or combination (CC coal-and-gas-fired) power plants (10) which capture a significant or majority fraction of their CO₂ emissions as SCF-CO₂ (11) which is piped underground (12) into porous rock structures for storage. Our FIG. 3 invention stage-1 will consider the prior-art-captured power-plant SCF-CO₂ (or liquid CO₂) as a major Type #1 prior-art contributor to stage-1 of an invention multi-stage system, in which the SCF-CO₂ (11) or the liquid CO₂ captured from the CC coal-fired and/or CC gas-fired electric power plants (10) is diverted from prior-art underground porous rock storage (12) to an above-ground invention stage-1 series (19) of multiple invention bioreactors (18), where the SCF-CO₂ or the liquid CO₂ is decompressed (14-17) and rapidly converted by invention-accelerated photosynthesis in bioreactors (18) to a particular form of high-density, heavier-than-water, fast-sinking marine seed algae at the collective (globally distributed) invention stage-1 rate of up to 3 GtC/yr of land-harvested salt-water algae (20). Details of one nonlimiting embodiment of the bioreactor which is an algae conversion silo (18) are given in FIG. 8. Examples of the high-density, fast-sinking marine seed algae produced (20) by the stage-1 invention bioreactors (18) would be coccolithophore (e.g., Emiliania huxleyi) or siliceous diatoms which are types of algae that are heavier-than-water, owing to a calcium carbonate or siliceous exoskeleton imparting specific gravity exceeding water to the algae. FIG. 6 illustrates that the up to 3 GtC/yr of stage-1 invention bioreactor seed algae (land harvest (FIGS. 3-5) of coccolithophore or siliceous diatom algae) may then be transported (FIG. 6) to sea-ports and widely dispersed (with micronutrients) at sea in stage-2 of our invention system to seed accelerated (much larger) ocean algal blooms of 14 GtC/yr, thereby imparting a substantially negative carbon footprint to stage-1 CC coal-fired, CC gas-fired, and/or combination CC coal-and-gas-fired power-plants (FIG. 3, items 10), using whole-earth carbon accounting. When combined with the up to 3 GtC/yr of land-harvested invention bioreactor (18, 65) seed (20), the FIGS. 6, 7 stage-2, 14 GtC/yr amplified ocean algal blooming will bring the total (land and sea) algal blooming rate to 17 GtC/yr (FIG. 7, curve 81), with that much CO₂ being captured as the combined algae bloom in the FIGS. 3-5 and 9-13 stage-1 invention bioreactors (18, 65, 90) and at sea (FIGS. 6, 7). The large negative carbon footprint arises in that up to 14 GtC/yr of CO₂ capture by the FIGS. 6, 7 stage-2 amplified ocean algal blooming was seeded by a fraction of the 1-3 GtC/yr of stage-1 land harvested seed algae (20, 82) produced, in part (FIG. 3), from the stage-1 CO₂ captured from the CC coal-fired and/or CC gas-fired power-plants (10). Triggered with stage-1 invention seed (1-3 GtC/yr) under invention-optimized conditions, nature will provide stage-2 ocean amplification and do the heavy lifting (14 GtC/yr) of extra CO₂ capture indicated in FIGS. 6, 7.

Further yet, FIG. 4 illustrates another embodiment of the Type #1 invention stage-1 which is an embodiment for making hydrogen (H₂) transportation fuel. FIG. 4 relates to prior-art natural-gas reformation conversion (33-37) of methane (30, CH₄) to H₂ (37), suitable for fueling hydrogen-powered vehicles (automobiles (38), vans, buses, trucks, planes, trains, boats, ships, etc.), in which an optimized combination natural-gas reformation process for hydrogen production (37) involves invention capture (39) of process byproduct CO₂ as SCF-CO₂ (40) or liquid CO₂ in a second Type #1 stage-1 invention embodiment (30-40) and imparts a substantial 1400% negative carbon footprint to natural-gas reformation hydrogen production by transferring the captured second Type #1 embodiment stage-1 natural-gas reformation process byproduct CO₂ to at least one (or multiple) invention bioreactors (18) where the reformation process byproduct CO₂ is rapidly converted by bioreactor (18) accelerated photosynthesis and/or coccolithogenesis (calcification) to the desired form of high-density marine seed algae (20) at a rate contributing substantially to the stage-1 land-harvest (20)—up to 3 GtC/yr total, the substantially (e.g., 1400% in a non-limiting example) negative carbon footprint being imparted to the natural-gas production of hydrogen (37) by the up to 3 GtC/yr of the stage-2 bioreactor seed algae being transported to sea-ports (FIG. 6) and widely dispersed (with micronutrients) at sea (FIG. 6) to seed the stage-2 accelerated ocean algal blooms of 14 GtC/yr (17 GtC/yr total land and sea CO₂ capture). The 1400% negative carbon footprint (whole-earth carbon accounting) arises in that up to 14 GtC/yr of CO₂ capture by the stage-2 amplified ocean algal blooming (FIG. 6) was seeded by a fraction of the 1-3 GtC/yr of land-harvested seed algae (FIG. 7, item 82) produced (in part) from the stage-1 natural-gas reformation process byproduct CO₂ (40).

Further yet, the multi-stage system relates to cement production in which an optimized Type #1 stage-1 invention captures cement production byproduct CO₂ as SCF-CO₂ or liquid CO₂ in a third embodiment (not shown) and imparts a negative carbon footprint to the cement production by transferring captured cement production byproduct CO₂ to the multiple invention bioreactors (18) where it is rapidly converted by the bioreactor accelerated photosynthesis and/or coccolithogenesis to the desired form of marine seed algae at a rate contributing substantially to the stage-1 land-harvest (up to 3 GtC/yr total), the substantially negative carbon footprint being imparted to the cement production by the up to 3 GtC/yr of the stage-1 invention bioreactor seed algae being transported to sea-ports (FIG. 6) and widely dispersed (with micronutrients) at sea to seed the FIG. 6 stage-2 accelerated ocean algal blooms of 14 GtC/yr. The negative carbon footprint (whole-earth carbon accounting) arises in that up to 14 GtC/yr of CO₂ capture by the stage-2 amplified ocean algal blooming was seeded by a fraction of the 1-3 GtC/yr stage-1 land harvest seed algae (FIG. 7, item 82) produced (in part) from cement production byproduct CO₂.

The multi-stage invention system further relates to capture of CO₂ from outdoor air, building flues, incinerators, crematoriums, kilns, blast-furnaces, refineries, factories, cement plants, power plants, natural-gas reformation systems, oil gasification systems and/or coal gasification systems in which additional invention Type #2 stage-1 embodiments are based on sodium hydroxide (NaOH, caustic soda, lye) capture of CO₂ from CO₂-laden gas mixtures as in FIGS. 9-12 or in which Type #3 stage-1 embodiments are based on an alkali bicarbonate or alkali carbonate or alkaline-earth carbonate solution starting point as in FIG. 13, the initial invention Type #2 or Type #3 embodiment stage-1 sodium bicarbonate, carbonate, or other alkali bicarbonate, carbonate, or alkaline earth carbonate solution being transferred to invention enclosed acidification chambers where CO₂ is released or re-released to one or more invention bioreactors (18, 65, 90) where it (CO₂) is rapidly converted by invention-accelerated photosynthesis and/or coccolithogenesis (calcification) to the desired form of high-density marine seed algae at a rate contributing substantially to the stage-1 land-harvest (up to 3 GtC/yr total), and in which a substantially negative carbon footprint is imparted to the outdoor air, building flue, incinerator, crematorium, kiln, blast-furnace, refinery, factory, cement plant, power plant, natural gas reformation system, oil gasification system, or coal gasification system by the up to 3 GtC/yr of the stage-1 invention bioreactor seed algae being transported (FIG. 6) to sea-ports and widely dispersed (with micronutrients) at sea to seed the FIGS. 6, 7 stage-2 accelerated (much larger) ocean algal blooms of 14 GtC/yr. The negative carbon footprint (whole-earth carbon accounting) arises in that up to 14 GtC/yr of CO₂ capture by the stage-2 amplified ocean algal blooming was seeded by a fraction of the 1-3 GtC/yr of the stage-1 land harvest seed algae produced (in part) from the Type #2 or Type #3 additional embodiment invention system stage-1 CO₂ captured from the outdoor air, building flues, incinerators, crematoriums, kilns, blast-furnaces, refineries, factories, cement plants, power plants, natural-gas reformation systems, oil gasification systems, or coal gasification systems.

In Type #2 embodiments of the multi-stage naturally amplified global scale carbon dioxide capture system, FIG. 9 illustrates that carbon dioxide separation and concentration may be achieved by invention reaction of CO₂-laden gas mixtures (120, 122) with sodium hydroxide (NaOH, caustic soda, lye (126-129)) in a thin film reactor (121) which functions as a lye scrubber, so that the CO₂ is captured by the downward flowing lye film (129) as sodium bicarbonate solution (130) which is then drained (130) and the CO₂ re-released by subsequent invention closed-system (139) acidification (131-133) of the bicarbonate solution (130) and inection of the re-released carbon dioxide (138, 140, 142) into the invention stage-1 bioreactors (algae conversion silos (18, 90)) where it feeds algal blooming to produce the stage-1 seed (20) for stage 2 ocean-amplified blooming (FIGS. 6, 7). One preferred embodiment of the FIG. 9 Type #2 land-based algal conversion—lye capture path for CO₂ is illustrated in FIG. 10 which is a home or filling station embodiment of hydrogen production (37) by methane reformation. This preferred embodiment captures CO₂ from the methane reformation process (150) in a thin film reactor (121) exposing the reformation gas mixture (122, 123) to a downward flowing lye film (129), capturing the spent reaction product bicarbonate solution (130), and storing it in a pickup vessel (151) for later transport to a district receiving station (162) which feeds the same acidification (131-133) and closed-system CO₂ re-release chamber (139) and land-based algae conversion silo (18, 90) as before. This embodiment also couples its silo algae output (20) to stage-2 (FIGS. 6, 7) for 15× ocean amplification as before. By this means, the FIGS. 10, 6 multi-stage invention imparts home or filling station hydrogen fueling of transportation with a 1400% negative carbon footprint, using whole earth carbon accounting. As in the case of FIG. 9, the FIG. 10 embodiment (with FIG. 6 ocean amplification) will contribute to the amplified CO₂ capture curves (2, 3, 81) of FIGS. 1, 7, but the invention boost to globalization of hydrogen-powered transportation will also lower emissions, contributing strongly to emissions reduction curve 1 (FIG. 1).

Other preferred embodiments of the FIGS. 9 and 10 land-based algal conversion type #2 (lye capture path) invention are illustrated in FIG. 11, which is a lye scrubber for home and building flues. It would work equally well for incinerators and crematoriums (not shown). It is once again based on exposing CO₂-laden flue gases (163, 166) in a rising vortex counter-flow (123) to a downward flowing lye film (129) produced by lye overflowing (128) a standpipe (127) contained within in a thin film reactor (121). If needed, auxiliary cooling air may optionally be mixed in with the hot flue gases (163, 166) prior to tangentially entering the thin film reactor (164, 167). The lye film (129) flowing down the outside of the standpipe (127) absorbs CO₂ from the rising vortex counter-flow of flue gases (123), converting the CO₂ to bicarbonate solution which then drains out of the reactor at 130. Stripped air (124) exits the thin film reactor at 168 and continues in the flue exhaust (170). If needed, flue gases may be pulled through the thin film reactor (121) with an exhaust fan (169) pulling on the stripped air (168) outlet. The bicarbonate collection vessel (151) of FIG. 11 may be considered a district pickup vessel like the pickup vessel (151) in FIG. 10 to be delivered to the district acidification system (131-140) and algae conversion silos (18, 90) of FIG. 10, and the silo output (20) may be further amplified by stage-2 operations at sea (FIGS. 6, 7). This system will impart 15× ocean amplification to land-based CO₂ capture from home and building flues, incinerators, and crematoriums, along with a 1400% negative carbon footprint, using whole earth carbon accounting. That amplified CO₂ capture will contribute strongly to capture curves 2, 3, and 81 of FIGS. 1 and 7, respectively, but there is also a flue-based emission reduction to be credited, which in turn contributes strongly to emission reduction curve 1 (FIG. 1).

One preferred embodiment of Type #2 land-based algal conversion is illustrated in FIG. 12 which is an outdoor air embodiment of Type #2 invention system CO₂ capture. It features a large scale invention bin (180) which houses a lye fountain through which large amounts of outdoor air are drawn. Air enters the lye fountain bin (180) through perimeter air intakes (182) around the base of the bin. The lye fountain is actually a flowing lye film (187) which absorbs CO₂ from the air to form sodium bicarbonate solution which exits spill-off drain (190), and enters the remainder of the Type #2 stage-1 invention system as in FIG. 9, 10, followed by substantial stage-2 capture amplification at sea (FIGS. 6, 7).

FIG. 12 shows one algae conversion silo (18, 90), but a cluster (not shown) may be envisioned in which each lye fountain bin (180) is surrounded by four algae conversion silos (18, 90) in a non-limiting example. Remediation parks containing, e.g. 48 of these clusters may be envisioned in a non-limiting example of high capacity outdoor air capture. Global proliferation of such remediation parks, perhaps as many as 20,000-200,000 parks in a non-limiting example and coupling of these parks to stage-2 invention ocean amplification (FIGS. 6, 7) will contribute to the FIGS. 1, 7 CO₂ capture goal (curves 2, 3, 81) of 17 GtC/yr contingency capture (curves 2, 81-FIGS. 1, 7) and 10 GtC/yr impact capture (curve 3-FIG. 1).

In FIG. 12, the lye fountain bin (180) houses a large, slow-rotating (e.g. ˜9 rpm in a non-limiting example—overhead motor not shown) air auger (181) which draws CO₂-laden air into the bin at perimeter intakes (182) located around the base of the bin. The auger (181) pushes air spirally up through the bin where it exhausts at the stripped-air exits (183). The air auger (181) drive shaft is hollow in a preferred embodiment. In one preferred non-limiting embodiment, the hollow shaft houses a smaller, higher speed auger (not shown) which draws lye solution from reservoir (184) into the hollow shaft (185) and propels it internally to the top where it spills out onto the upper extent of the large slow-moving air-auger blades (186). The lye solution spreads over the auger blades covering them with a lye film (187) of high surface area which runs down the blades in a film, flowing counter to the rising air column being pushed upward by the blades. Gravity draws the lye film (187) downward over the blades as blade rotation pushes the air upward. This is an efficient, high surface area film reactor in which the rising spiral flow of air interacts with the downward spiral (film) counter-flow (187) of lye solution. The downward flowing lye film (187) absorbs CO₂ from the air as it passes spirally upward through the bin and the lye film may be quantitatively converted to sodium bicarbonate solution which spills off the bottom of the auger blades at 188, hits a sloping false bottom (189) in the bin, and exits via the indicated sodium bicarbonate (NaHCO₃) drain (190). From there, the sodium bicarbonate enters the remainder of the stage-1 invention algal conversion system as in FIGS. 9, 10, followed by substantial stage-2 capture amplification at sea (FIGS. 6, 7).

In Type #3 (NaHCO₃ starter) embodiments of the multi-stage naturally amplified global scale carbon dioxide invention capture system, FIG. 13 illustrates that any generic source of carbonate or bicarbonate solution resulting from CO₂ capture may be processed by subsequent invention closed-system acidification of the bicarbonate solution and infusion of the re-released carbon dioxide into the headspace of invention stage-1 bioreactors (algae conversion silos) where it feeds algal blooming to produce the stage-1 seed for stage-2 ocean-amplified blooming (FIGS. 6, 7).

Further yet, the multi-stage invention system relates to capture of CO₂ from outdoor air, building flues, incinerators, crematoriums, kilns, blast-furnaces, refineries, factories, cement plants, power plants, natural-gas reformation systems, oil gasification systems, or coal gasification systems, in which a final group of invention stage-1 embodiments are based on any means of CO₂ capture (including prior-art stage-1 capture means with invention diversion of captured CO₂ to invention stage-1 holding stations or reservoirs or invention stage-1 processing stations) in which the any means of CO₂ capture yields relatively concentrated CO₂ as a gas, liquid, super-critical fluid, carbonate solution, or bicarbonate solution, and in which the final-group invention multi-stage embodiments impart a negative carbon footprint to the outdoor air, building flue, incinerator, crematorium, kilns, blast-furnaces, refineries, factories, cement plants, power plants, natural-gas reformation systems, oil gasification systems, or coal gasification systems by transferring the captured final-group embodiment stage-1 outdoor air, building flue, incinerator, crematorium, kiln, blast-furnace, refinery, factory, cement plant, power plant, natural-gas reformation system, oil gasification system, or coal gasification system, relatively concentrated CO₂ to the multiple invention acidification sections and/or bioreactors (18, 65, 90) of FIGS. 3-5 and FIGS. 8-13 where the transferred CO₂ is rapidly converted by the invention accelerated photosynthesis and/or coccolithogenesis (calcification) to the desired form of high-density marine seed algae at a rate contributing substantially to the (e.g., FIG. 5) stage-1 land-harvest (up to 3 GtC/yr total), and a substantially negative carbon footprint being imparted to the outdoor air, building flues, incinerators, crematoriums, kilns, blast-furnaces, refineries, factories, cement plants, power plants, natural-gas reformation systems, oil gasification systems, or coal gasification systems by the up to 3 GtC/yr of the stage-1 invention bioreactor seed algae being transported to sea-ports (FIG. 6) and widely dispersed (with micronutrients) at sea to seed the stage-2 accelerated (much larger) ocean algal blooms of 14 GtC/yr. The negative carbon footprint (whole-earth carbon accounting) arises in that up to 14 GtC/yr of CO₂ capture by the stage-2 amplified ocean algal blooming was seeded by a fraction of the stage-1 land harvest seed algae produced in part from the final-group embodiment stage-1 CO₂ captured from the outdoor air, building flues, incinerators, crematoriums, kilns, blast-furnaces, refineries, factories, cement plants, power plants, natural-gas reformation systems, oil gasification systems, or coal gasification systems.

Stage-1 land-based capture includes arrays of at least one high capacity invention algae bioreactor (FIGS. 3-5 and FIGS. 9, 10, 12, 13, items (18, 65, 90)) to continuously convert relatively concentrated CO₂ from prior-art and/or invention preliminary capture system(s) to high density, fast-sinking, marine algae on land, essentially as fast as the preliminary capture systems capture CO₂. This will require acceleration of photosynthesis and/or coccolithogenesis (calcification) in the at least one high capacity invention algae bioreactor (18, 65, 90).

Referring to FIG. 8 in a nonlimiting example, the acceleration of photosynthesis in the at least one high capacity invention algae bioreactor (90) will be due in part to the high concentration of CO₂ introduced (91, 92) into the stage-1 bioreactor headspace. In comparison to today's ambient CO₂ level of 400 ppm (0.04%), the stage-1 bioreactor (algae conversion silo) headspace will be infused with sufficient CO₂ to maximize algal blooming rates. This could be up to 100% CO₂ in a non-limiting example, but the optimal amount will likely be lower than that, and in any case it will be easily adjustable to optimized intermediate levels (e.g. 1%-50% CO₂ in non-limiting examples) to maximize the algal blooming rate at any selected seed, nutrient, light level, and illumination wavelength at a given bioreactor operating temperature, while minimizing acidification (carbonation) of the algae pool (94). To prevent or substantially offset carbonation by the high concentration of headspace CO₂ acidifying the algae pool (94, dissolving or softening coccolithophore calcareous exoskeletal coccolith plates), the pool will be buffered at approximately pH 8.32 in a non-limiting example. In this non-limiting example, pH buffering at pH 8.32 will achieved by adding a solution mixture of disodium phosphate and monosodium phosphate in a mole ratio of approximately thirteen-to-one, respectively, and in which the phosphate buffering components also double as photosynthesis micronutrients to support algal blooming. If phosphate depleted nutrients are desired to alleviate phosphate supply shortages and/or to further enhance species-selective bloom dominance in stage-2 ocean blooming, then buffer mixtures other than phosphate salts (e.g., a borate buffer system, in a nonlimiting example) would have to be substituted.

Photosynthetic and/or coccolithogenic (calcification) acceleration (accelerated algal blooming) will be due in further part to exceptionally high seed levels of the coccolithophore or siliceous diatom algae introduced into the invention bioreactor algae pool (94), the seed levels for constant blooming in the invention bioreactor being unusually high—up to 15% solids (by weight) in a non-limiting example, and this will radically accelerate blooming by continuously operating the bioreactor exceptionally high on the (upward-bending) nonlinear growth curve. Normally, this solids level would exceed optical opacity limits and photosynthesis could not proceed, owing to lack of light penetration, however a novel invention optical thinning effect (see below) will circumvent prior-art opacity limits.

In FIG. 8, unusually high seed levels in the algae pool (94) will be enabled by an invention optical thinning effect produced by the vertical rotary auger (95) which lifts algae suspension continuously out of the pool and slings it off the edges of the auger blades continuously throughout most of the height of the bioreactor, creating an inter-twined helical sheet fountain of algae suspension. The sheets of algae suspension slinging continuously off the exposed (non-submerged) edges of the auger blade (95) will be thin fountain sheets and will produce an optical thinning effect which allows overhead light (96) penetration to a degree far exceeding that of the concentrated algae pool (94) below. Light penetration through the optically thinned fountain sheets will activate photosynthesis in the seed algae, activating it as it falls back into the pool or hits the side wall of the reactor and runs down into the pool, where the auger lifts it and slings it in sheets, over and over again. With the optically-thinned fountain sheets, exposed surface area of the seed suspension is exceptionally high and light penetration into (and through) the thin fountain sheets will be exceptionally good, driving prodigious algal bloom rates continuously and permitting much higher % solids levels to develop, well beyond that otherwise permitted by the opacity of the pool (94) below. This will allow much higher seed levels and also much higher harvest bloom levels than could otherwise be achieved in a pool reactor (94) alone. 15% seed levels will become feasible in this invention. That is very high on the nonlinear growth curve and it will drive prodigious blooming as a result. Mechanical shear from the auger blades will prevent colonization from occurring and it will keep the algae suspension free-flowing (non-agglomerated), despite the high solids level (15% in a non-limiting example) in the suspension and despite prodigious bloom rates.

A second smaller transfer auger (not shown) will be turned on and operated to continuously remove algae suspension from the bioreactor as fast as it blooms (in excess of 15% solids). In one non-limiting embodiment, the funnel shaped silo floor would enable excess bloom removal at outlet 99. The concept here is that high seed levels (15% solids) drive very high bloom rates, but outlet 99 removal of excess bloom from the bioreactor occurs as fast as it develops, leaving a constant seed level of 15% solids behind in the reactor. This is a continuous reactor which doesn't require reseeding, once the solids level reaches 15% and the transfer auger (not shown) is turned on to keep it from going higher by continuously removing excess bloom at 99. As excess bloom is removed from the bioreactor (99), water, buffer, and nutrient are continuously replenished (21), but no new algae seed is required—enough seed remains behind from the bloom, if the transfer auger removal rate (99) is balanced exactly at the bloom level and it isn't turned on until the bloom level first reaches 15%. The transfer auger then removes excess bloom continuously (as fast as it develops), without diminishing the 15% solids level, which then becomes the continuous seed level.

The transfer auger removes 15% algae suspension to an adjacent separation tank (100). The separation tank (100) is relatively large diameter to cause a significant reduction in flow velocity at the same flow rate as 101. This velocity reduction is important, because it suddenly offers the tiny algae (e.g. 2 μm in diameter and having flagella for motility in a nonlimiting E. huxleyi example) an opportunity to swim against the current, if they so desire. What is needed next is a reason for the algae to swim against the current so that they will concentrate in the upper end of the separation tank. That impetus is provided by tank (100) and its main downward flow path being dark and essentially devoid of both CO₂ and nutrient, whereas an attractant light beam (beacon 106, 107) is positioned within the mouth of a harvest exit tee (105) located near the upper extent of tank (100).

With the main separation tank volume (100) and path (101→102) being essentially devoid of light, and with the flow velocity significantly reduced at large tank diameter, the algae may swim against downward current (101→102)—swimming upward instead toward the attractant beacon (107) and illuminator globe (106) supplied at the mouth of the harvest exit tee (105). The exit tee and harvest exit path (105→20) are smaller in diameter again and, even though the exit path (105→20) flow rate is low, this diameter reduction raises flow velocity (relative to path 101→102) enough that any algae which appear at the mouth of the exit tee (106, 105) will be sucked into harvest exit flow path (105). Marine algae may be continuously harvested as ocean seed at the harvest output of the silo (20). The bioreactor is continuous, self-concentrating, and will promote prodigious algal blooming at output (20). About 85% of the algal bloom will continuously exit via the harvest path (105) in a nonlimiting example, with about 15% recirculating via path (102-104). Any dead algae will sink and may be periodically removed at (109).

In an alternate embodiment, heavier-than-water algae from the bioreactor may proceed to an adjacent settling tank after blooming, in which the settling tank replaces the aforementioned separation tank; and in which settling tank conditions are maintained that do not encourage algae to swim against a current, and in which the heavier-than-water algae instead sink toward a funnel shaped harvest exit port at the bottom of the settling tank, and in which optional recirculation of clarified liquid near the top of the settling tank is provided back to the main bioreactor, with top-water clarification occurring as the algae sink to the funnel shaped bottom, and in which a concentrating effect is achieved via sedimentation of the sinking algae prior to their exit at the harvest exit port.

In both embodiments, a pH buffer (e.g., phosphate buffer, in a nonlimiting example) added (21) to the algae pool (94), buffers the pool against acidification (carbonation) from high level headspace CO₂. Buffering the pH at nominally 8.32 will maximize coccolithophore algae blooming and prevent softening or acidic dissolution of the coccolithophore exoskeleton (CaCO₃). As algae is continuously harvested (20) as a concentrated suspension, replenishment sea water or salt water, nutrient, and pH buffer are provided at the replenishment inputs (21) to the silo algae pool (94).

Oxygen produced during photosynthesis is continuously removed by an oxygen removal system (119, 110-116) based on at least one oxygen-permeable membrane (116), which is tubular in the nonlimiting FIG. 8 embodiment, and a far-side exhaust sweep gas (113), such as nitrogen (112) in a non-limiting example. A tubular membrane (116) and far-side annular sweep gas space (113) are depicted in this non-limiting example. Only one oxygen removal system (119) is depicted, but multiple units (of 119) mounted on the same silo would also be within the scope of the invention. In this oxygen removal system (119), a fraction of the silo headspace gas would be drawn by fan (not shown) into the removal system at 110 and down through the removal system center (115). Oxygen in the mixture would selectively permeate membranes (116) into a nitrogen sweep gas (113) introduced at 112. The nitrogen sweep gas (113) would remove all of the permeating oxygen and exhaust it at 113A. CO₂ in the mixture would continue down the center (115) and wouldn't permeate the tubular membrane. It would simply rejoin the silo headspace at 111, just above pool 94.

This stage-1 invention bioreactor system (90) may be considered a pseudo-anaerobic bioreactor since oxygen is removed (119) as fast as it is produced by photosynthesis. Algal blooming will therefore proceed under pseudo-anaerobic conditions which will enhance bloom rates, because oxygen otherwise acts as a photosynthetic inhibitor (above a certain point), and its continuous removal (119) will accelerate blooming.

If sufficient numbers of these FIG. 8 stage-1 algae bioreactors are globally proliferated for processing concentrated CO₂ in the invention embodiments of FIGS. 3-5 and FIGS. 9, 10, 12 and 13, the collective harvest rate of high-density, marine seed algae shipping to sea-ports for transfer to invention stage-2 (FIG. 6, operations-at-sea) can reach 3 GtC/yr.

Stage-2 of the multistage capture system involves FIG. 6, operations-at-sea. The stage-2 invention concept is to use stage-1 land-harvested high-density, fast-sinking marine algae (70, 72) (e.g., coccolithophore or siliceous diatom algae in two non-limiting examples) to selectively seed stage-2 (71), 15× amplified blooms of the same algae at sea, yielding 14 GtC/yr ocean blooms, and capturing that much atmospheric CO₂ (at sea) in the process. If stage-1 (70) captures 3 GtC/yr of CO₂ in all of its various FIG. 3-5 and FIG. 9-13 embodiments and the stage-1 bioreactors (18, 65, 90) convert that to high-density, marine algae (e.g., coccolithophore or siliceous diatoms in two non-limiting examples), and that is widely dispersed in invention stage-2 across 70% of Earth's oceans (FIG. 6), 2 GtC/yr of the land-harvested seed will satiate ocean grazer (e.g. copepods and krill) appetites leaving 1 GtC/yr uneaten to seed stage-2 15× amplified ocean blooming to yield 14 GtC/yr of ocean bloom, capturing 14 GtC/yr of atmospheric CO₂ as it blooms, then the total annual capture rate (land and sea) will be 17 GtC/yr CO₂ (FIG. 7) which satisfies the original combination invention capture targets (curves 2, 3, FIG. 1).

To accomplish all of that, FIG. 6 illustrates that up to 3 GtC/yr of high-density salt-water algae may be transported from land-based stage-1 bioreactors in specially designed stasis-supporting cargo containers (73) by flat-bed truck, rail, and barge to seaports where the containers would be loaded onto ocean-going freighters for wide distribution to floating repositories (74) in the open sea. From the floating repositories, the containers would be loaded onto fleets of smaller seed boats (75) which fan out from the repositories and dispense the seed (and micro-nutrient) directly from the containers into alternating “seed lanes” stretching across 70% of the oceans (76).

The invention cargo containers (73) would be stasis-supporting. In a non-limiting example, they would have a power source, built-in chillers to lower temperature to a stasis-inducing level in hot climates (or heaters in cold climates), enough nutrient (and just enough light) to keep the seed alive in stasis, and a slowly churning auger to prevent the seed from colonizing (agglomerating). The containers may be transferred by crane from flat-bed trucks to inland docks, from inland docks to flat-rail cars or barges, from rail-cars or barges to seaport docks, from seaport docks to ocean freighter decks and holds, from ocean freighter decks and holds to floating repository decks, and from floating repository decks to individual seed boat decks. Each of the aforementioned transfers can easily be made by large fork lifts, dock cranes, or deck cranes and the containers will maintain stasis-support at all stages of shipment and transfer, until the seed is dispensed into the ocean sea-lanes for enhanced stage-2 blooming.

Dispensing of seed and nutrient into sea-lanes from the seed boats will be at a measured rate while the boat is moving. In a non-limiting example, seed levels would be at least 20 mg/m³ in alternating sea lanes which are nominally 60 feet wide and 10 meters deep, which FIG. 2 suggests would be higher than the average natural algae levels occurring across most of the oceans south of Spain, Japan, and Seattle. This will give our high-density fast sinking seed algae a competitive advantage (among natural algae species) regarding nutrient, and ocean blooming will be dominated by the desired high-density, fast-sinking marine algae of stage-1 silo harvests (20, 72—FIGS. 5, 6), which is being seeded into the ocean in stage-2.

In one embodiment of stage-2 operations-at-sea, alternating sea lanes will be temporarily deaerated to a depth of 10 meters (in a non-limiting example) by bubbling N₂ behind the seed boat as the seed and nutrient are dispensed. This will temporarily displace dissolved oxygen (but not dissolved CO₂ (normal level maintained by the excess bicarbonate content of the sea)) to a depth of 10 meters (only) and a pseudo-anaerobic condition will be temporarily created in each localized sea-lane being seeded. The pseudo-anaerobic condition may accelerate blooming, especially if the algae seed are nitrogen-fixing. Adjacent lanes will be seeded two weeks out of phase with one another, so that the pseudo-anaerobic condition is both transient and localized (beneficial, rather than harmful).

Micro-nutrient will be dispensed in metered doses to support only about a 2 week bloom in each sea-lane. With the high seed level (e.g., at least 20 mg/m³) inherent with invention stage-2 seeding “algae +micro-nutrient” (in contrast to prior-art systems which dose “micro-nutrient-alone” and start their bloom from a much lower point (e.g., 0.1 mg/m³, per FIG. 2)), prodigious invention stage-2 bloom rates will occur, reaching the light penetration limit (−400 mg/m³ in a nonlimiting example) within about 2 weeks in alternating lanes.

Grazers may eat up to ⅔ of the seed before it blooms, but that is the reported limit of their appetites at this seed level, so ⅓ should remain to bloom to the light penetration limit within 2 weeks. At this point the metered micro-nutrient doses are calculated to run out and the bloom will die. The important point is that the invention bloom is dominated by high-density algae which will lose motility (post mortem), sink, and easily clear the photic zone in time for next month's reseeding. Thus, the invention stage-2 operations-at-sea will enable 12 large ocean blooms per year, instead of just one or two blooms which is the limit of prior art systems which dose nutrient-only, start at a much lower point on the growth curve, are subject to getting eaten out (before blooming) by grazers, and even if prior-art systems could get past the grazers (which they can't), they'd bloom up buoyant strains of algae that don't sink (post mortem) or clear the photic zone at the end of a bloom cycle. A persistent floating light-block would prevent a second bloom from occurring with prior-art ocean fertilization, which will generally bloom buoyant strains of algae rather than (preferred) high-density, fast-sinking strains. Prior-art ocean fertilization systems (dosing micro-nutrient-only) would, under the most favorable of conditions (where grazers don't interfere—but not much chance of that happening) yield 1 or 2 blooms/year, capturing about 1.5-3 GtC/yr CO₂ at best.

(Note: Even natural ocean blooming during the ice-ages would have been limited by grazers and the light penetration limits imposed by buoyant natural strains, but stage-2 invention ocean blooming won't be subject to these limits.)

In contrast, the multi-stage invention system which starts higher on the nonlinear ocean algae growth curve (by seeding algae+micro-nutrient), pre-satiates grazer appetites (2 GtC/yr) so there will remain 1 GtC/yr of (net) uneaten seed remaining to bloom (after grazer feasting), and which selectively blooms only the high-density, fast-sinking strains of coccolithophore or siliceous diatom algae (seed selectively pre-grown in stage-1 bioreactors) at sea will capture a total of 17 GtC/yr to meet the Curves 2, 3, and 81 targets of FIGS. 1, 7, respectively, while unsuccessful prior-art ocean fertilization attempts continue to languish at the mercy of grazers, slow bloom rates, and persistent floating light blocks which will limit their capture capacity to a maximum of about 1.5-3 GtC/yr, and often much less than that as grazers devour what little natural seed they have (e.g., PolarStern, 2009). Note that 1.5-3 GtC/yr blooming is substantially less than current and projected global emissions of 10-12 GtC/yr, so “nutrient-only” fertilization cannot offset emissions or avert 450 ppm CO₂ tipping points, or meet the targets of FIG. 1.

The above-listed invention system enhancements are anticipated to accelerate stage-2 ocean blooming significantly beyond the ice-age blooming rates. We project acceleration will be enough to enable 12 blooms/yr and meet the performance required by curves 2, 3, and 81 of FIGS. 1 and 7.

In one embodiment of an invention stage-2 ocean capture process, aerator boats will bubble compressed air or oxygen to within 5 meters of the sea floor in coastal waters to reaerate the lanes at the end of each monthly bloom cycle and prevent proximal post-bloom anoxia (which would otherwise greatly harm coastal marine life and raise legal objections with prior-art ocean fertilization attempts). Anoxia is typically a coastal water phenomenon which isn't prevalent in the open sea, where most of our stage-2 seeding will be done. In the open sea, re-aeration shouldn't be necessary, species-selective bloom dominance and use of heavier-than-water stage-1 algae seed will enable rapid sinking each month, sinking the dead algae quickly below the deep ocean thermocline and all the way to the cold deep sea floor, before anoxia has any chance of developing. Low deep ocean floor temperatures approaching zero degrees centrigrade and heavy coccolith plates should further delay the onset of bacterial action that could otherwise induce post-bloom anoxia. Delay may occur until sedimentation burial eliminates any further chance of developing anoxia. The localized, transient nature of invention system induced algal blooming and marine life feeding on the dead algae on the way down or at the sea floor may further suppress anoxic development.

If the 17 GtC/yr total multi-stage CO₂ contingency capture rate and 10 GtC/yr impact capture (FIGS. 1, 7, curves 2, 3, and 81, respectively) are collectively achieved by the FIGS. 3-13 invention embodiments and the invention-system-enhanced emissions cap and reduction curve 1 (FIG. 1) is concurrently achieved, then the final atmospheric accumulation impact curve 6 of FIG. 1 will successfully avoid the impending, near-term 450 ppm CO₂ tipping points and subsequently restore the pre-industrial level of 280 ppm CO₂ (9) by 2075. That will eliminate ocean acidification and set the stage for subsequent warming reversal (following a thermal lag delay), which is the goal of this multi-stage, multi-faceted invention system.

In addition to the invention bioreactors contributing significantly to climate restoration and ocean revitalization, other applications will include high capacity algal production for silage, animal feed, feed supplements, fertilizer, biofuels, agricultural runoff control, food for fish and seafood farming involving fish or mollusks which directly feed on algae, and bottom-rung food for fish farming involving predator fish (as seafood) such as compano and cobia which feed on lower marine life (e.g, brine shrimp). In the latter case, invention high capacity algal production will feed the brine shrimp in adjacent tanks, raising shrimp for secondary feeding to predator fish.

In these other applications, the algae silos (18, 65, 90) would be used seed species optimized for silage, animal feed (or supplement), fertilizer, biofuel, agricultural runoff control, or food for fish and seafood farming and the bioreactor output (20) would be directed to those applications which end with stage-1 without sending algae for stage-2 (FIG. 6). If desired the bioreactor output (20) may be additionally filtered and/or dried to remove the suspension water and excess nutrients before transferring algae to the land-based feed applications.

Using invention bioreactors along inland lake shores and rivers, invention fresh-water algal production can further aid in revitalization of inland lakes and rivers by removal of nitrogen and phosphorus compounds added by agricultural runoff. This would be accomplished by diverting the bioreactor output (20) directly into the lake or river. In this case, it would be desirable for the bioreactor algae to be a high density, fast sinking variety of fresh water algae. The algae bloom need not be supplemented with nutrient as it is dosed into the lake or river. As the algae bloom proceeds in lakes and rivers, it will consume nutrient provided by agricultural runoff, and in doing so, it will clear the river of these agricultural pollutants. As the algae blooms die and settle to the lake or river bottom, some periodic dredging may be required to keep the main channels open and an aerator boat may need to patrol up and down the rivers and on the lakes to restore dissolved oxygen levels to prevent post-bloom anoxia as algae blooms die and sink. With re-aeration, inland freshwater algae blooms will be beneficial as they will feed the lake and river food chain and increase fresh-water fish populations which will also flourish (and be healthier for fresh-water fishermen to catch and eat) as agricultural runoff chemicals are removed.

Lake and river bacteria levels will also drop sharply as another benefit of this program. This will improve the health of fish, water birds, and essentially all creatures and humans living in or along the lakes and rivers. This includes impacting water-borne disease, the eradication or minimization of which will benefit 3^{rd} world countries.

Clearing major rivers of agricultural runoff and bacteria will improve public health and will further stop coastal water harmful algae blooms (HAB's) such as the notorious “red tide” in Florida, which are otherwise fed from agricultural runoff at major river delta outflows. This will be accomplished by the invention high density fresh water algae having cleared the rivers of agricultural phosphorus and nitrogen compounds upstream from the delta outflow. The coastal water HAB's will simply die as their food supply will have been cut off upstream in the rivers which normally supply them with agricultural runoff. By clearing up the agricultural runoff, downstream HAB's in the gulf won't survive. By these invention means, lakes, rivers, and coastal waters will be revitalized. Even the tourism industry around lakes, rivers, and coastal waters will benefit as a result of better fishing everywhere with larger populations of bigger, healthier fish which are safer to eat as a result of growing in the cleaner, less polluted water.

The specification figures and description are of non-limiting examples and the invention systems and processes may be envisioned beyond the scope of specific embodiments, settings, and regions described herein, and the scope of the invention must therefore be considered to be limited only by the claims. While the invention system and processes have been described in terms of preferred embodiments, those skilled in the art will recognize that the invention can be practiced with modification within the spirit and scope of the appended claims.

1. A system for production of algae, the system comprising:
a CO₂ source; and
a bioreactor supplied with concentrated CO₂ from the CO₂ source, the bioreactor configured to encourage accelerated growth and reproduction of algae as well as to enable development of a more concentrated final algal bloom;
in which optical opacity limits on seed level and bloom concentration are circumvented by an optical thinning effect which enables greater light penetration into more concentrated algae suspensions;
wherein the greater light penetration enables higher level initial seeding or inoculation of the bioreactor bloom space;
wherein the higher level of initial seed accelerates blooming as a result of starting higher on an upward-bending nonlinear algal growth curve;
and in which a normally inaccessible upper section of the nonlinear algal growth curve is conventionally inaccessible owing to optical opacity of concentrated algal suspensions;
and in which the normally inaccessible upper section of the nonlinear growth curve is rendered accessible by the optical thinning effect which enables light penetration into optically thinned suspensions of concentrated algae.

2. The system of claim 1, wherein the optical thinning effect is produced by slinging an algae suspension as thin watery sheets off the perimeter edges of a rotating auger blade which lifts algae suspension out of a pool, elevates the lifted suspension, and slings it outward by centrifugal force to form optically thin watery sheets,
and wherein optical thinness of the slinging sheets enables improved optical penetration by rays from a light source shining through the slinging sheets.

3. The system of claim 1, in which the algae suspension from the bioreactor proceeds to a flow-through separation tank after blooming,
wherein the flow velocity of algae suspension through the separation tank is reduced, at constant flow rate, by means of enlarged tank diameter,
and wherein the reduced flow velocity is low enough to permit algae that have flagella or other motility means to swim effectively against the flow current when presented with an upstream or side-stream attractant,
and wherein the direction of algal swimming is toward the attractant,
and wherein algal swimming toward the attractant produces a concentrating effect on the algal suspension,
and wherein the concentration of algae proximal to the attractant is made higher by the concentrating effect than the concentration of algae at points located progressively downstream from the attractant and still within the main flow of the flow-through separation tank.

4. The system of claim 3, wherein the separation tank contains a main flow exit port and a secondary exit port which is designated as a harvest exit tee,
wherein the attractant is located at a position proximal to the mouth of the harvest exit tee,
and wherein the mouth of the harvest exit tee is sufficiently narrow to raise the harvest exit flow velocity to exceed the capacity for algae to swim against the harvest exit current,
wherein algae swimming toward the attractant from the main separation tank are sucked into the harvest exit tee upon reaching the attractant,
wherein the harvest exit tee outflow leads to an algal harvest output port,
wherein the concentration of algae harvested at the harvest output port is higher than the concentration of algae entering the separation tank,
and wherein the main flow of the flow through exit tank at points downstream of the attractant and having bypassed the harvest exit tee contains a reduced concentration of algae, relative to the concentration of algae entering the separation tank,
and wherein the main flow of the flow through exit tank having bypassed the harvest exit tee exits the separation tank through the main flow exit port,
and wherein flow exiting the main flow exit port is recirculated to the original bioreactor.

5. The system of claim 4, in which the attractant is one or more attractants selected from among a group of attractants consisting of a light source, a nutrient source, a carbon dioxide source, an attractive water temperature, and an attractive water pH,
and wherein the rest of the separation tank is dark and relatively devoid of the chosen attractant or combination of attractants.

6. A system for production of algae, the system comprising:
a hydrocarbon cracking reactor configured to generate a stream of concentrated CO₂ byproduct; and
a bioreactor configured to produce heavier-than-water algae, the bioreactor supplied, at least in part, with CO₂ from the stream of concentrated CO₂ byproduct;
wherein the hydrocarbon cracking reactor produces H₂ as its main product.

7. The system of claim 6, wherein the hydrocarbon cracking reactor is a two-stage steam reactor operating with steam stages at two different temperatures, optimized for cracking methane as the principal component of natural-gas.

8. The system of claim 1 wherein the CO₂ source is a CC (carbon-capture) clean-coal-fired power plant, the CC power plant producing electricity as a public utility and concentrated CO₂ byproduct as the CO₂ source in the form of a supercritical fluid (SCF-CO₂).

9. The system of claim 8, wherein the SCF-CO₂ is decompressed to concentrated CO₂ gas and introduced into the bioreactor.

10. The system of claim 1 wherein the CO₂ source is a CC (carbon-capture) gas-fired power plant, the CC power plant producing electricity as public utility and concentrated CO₂ byproduct as the CO₂ source in the form of a supercritical fluid (SCF-CO₂).

11. The system of claim 10, wherein the SCF-CO₂ is decompressed to concentrated CO₂ gas and introduced into the bioreactor.

12. A process of ocean-amplified CO₂ capture,
wherein algae plus nutrient are seeded into the ocean instead of nutrient-alone; the process comprising:
land-based capture of concentrated CO₂ from a land-based CO₂ source;
land-based conversion of captured CO₂ to heavier-than-water marine algae in at least one bioreactor configured to encourage the rapid growth and reproduction of the heavier-than-water marine algae as ocean seed;
transport of the heavier-than-water marine algae as ocean seed to seaports for ocean distribution and dispersal with added micro-nutrients in order to seed ocean-amplified blooming (further growth and rapid reproduction at sea—essentially secondary blooming on a vast ocean scale);
wherein the ocean-amplified blooming occurs essentially selectively for the heavier-than-water species of marine algae by virtue of the heavier-than-water marine algae being distributed, dispersed, and seeded into the ocean water at higher levels than existing natural buoyant ocean algae, the higher levels selectively accelerating ocean blooming rates of the heavier-than-water marine algae by virtue of seeding the ocean with marine algae seed harvested from the at least one land-based bioreactor,
wherein ocean seeding occurs higher than normal on a nonlinear algal growth curve and produces a species-selective dominance of the ocean algal bloom,
wherein the higher that the ocean blooming starts on the growth curve, the faster it proceeds, if sufficient nutrient is present or provided.

13. The process of claim 12 in which the species-selective ocean algal bloom dominance is further enhanced by nutrient selection,
and in which nutrient selection for E. huxleyi coccolithophorid marine algae blooming includes nutrients which are deficient in phosphate,
wherein phosphate deficiency, while other nutrients are concurrently provided in abundance, promotes prodigious E. huxleyi growth at sea, essentially to the exclusion of blooming by other species of marine algae, including buoyant algae, in the seeded ocean area.

14. The process of claim 12, wherein transport to seaport of the heavier-than-water marine algae seed occurs by flat-bed truck, flat rail car, or barge;
wherein the flat-bed truck, flat rail car, or barge carry the marine algae seed in stasis-supporting cargo containers which are transferrable by crane or other lifting means from one flat-bed transportation means to another, and
wherein the cargo containers are designed to maintain conditions in support of a healthy stasis condition for the heavier-than-water marine algae seed.

15. The process of claim 14, wherein the stasis-supporting cargo containers may be loaded onto ocean freighters docked at seaports, the ocean freighters then distributing the stasis-supporting cargo containers to floating seed repositories at sea;
wherefrom the stasis-supporting cargo containers may be transferred to seed dispersal boats which fan out from the floating seed repositories to disperse and dispense the heavier-than-water marine algae seed (plus micronutrients) into the ocean for ocean-amplified blooming to proceed, along with ocean-amplified CO₂ capture as the heavier-than-water marine algae bloom prodigiously at sea.

16. The process of claim 15, wherein the micro-nutrient doses are metered to support heavier-than-water ocean-amplified algal blooming up to the light penetration (algal opacity) limit and then run out.

17. The process of claim 16, wherein the ocean-amplified bloom dies after the metered micro-nutrient doses run out;
and wherein the dead heavier-than-water amplified bloom loses motility and sinks rapidly, clearing the ocean photic zone before the end of each month and enabling restored light penetration into the photic zone to support another amplified bloom following a next month's seeding.

18. The process of claim 17 in which up to 12 blooms/year may be seeded and achieved, with each ocean-amplified bloom reaching the light penetration (algal opacity) limit before it dies and sinks,
and in which accumulated amplified ocean blooming yields up to 14 GtC/yr of heavier-than-water algae (correspondingly capturing 14 GtC/yr of atmospheric CO₂) globally for each 1-3 GtC/yr of seeding with land-based heavier-than-water algae seed produced by the land-based bioreactors,
wherein the predominant heavier-than-water ocean algal bloom species are determined by the species of land-based bioreactor seed algae harvested from the bioreactor,
and wherein the bioreactor seed algae are dominated by initially preseeding the bioreactor with a purified culture of the desired marine algae species,
and wherein the desired marine algae species are selected from a group consisting of coccolithophore (e.g., E. huxleyi) and siliceous diatoms.

19. The process of claim 17, wherein the seeding of amplified ocean blooming is restricted to the vast open ocean that is further out from shore, well beyond the realm of coastal waters and beyond the shallow coastal-shelf sea floor, out in the open seas where much deeper water prevails,
wherein species-selective bloom dominance and rapid sinking quickly carries the dead heavier-than-water algae below the ocean thermocline of the open seas and all the way to the deep-sea floor,
wherein deep ocean temperatures at the deep-sea floor are quite low—near to zero degrees centrigade,
and wherein low deep-sea temperatures preserve the dead algae and slow and/or suppress the onset of secondary bacterial action, algal decay, eutrophication, and post-bloom anoxia which would otherwise deplete ocean-dissolved oxygen,
and wherein the slowing or suppression of bacterial action at low temperature at the deep-sea floor delays the onset of eutrophication and post bloom anoxia to an extent enabling ocean sedimentation, often referred to as marine “snow”, to essentially bury the dead algae before significant post-bloom anoxia or eutrophication can develop.

20. The process of claim 18, wherein approximately 1 GtC/yr of seed triggers amplified ocean blooming of up to 14 GtC/yr of heavier-than-water algae;
but wherein approximately another 2 GtC/yr of seed are needed to satiate marine grazer appetites so that they leave the approximately 1 GtC/yr of seed uneaten so that it remains to trigger the amplified ocean blooming of the up to 14 GtC/yr of heavier-than-water algae and corresponding photosynthetic and/or coccolithogenic (calcification) capture of up to 14 GtC/yr of atmospheric CO₂,
and in which ocean seeding with approximately 3 GtC/yr of algal seed produced by land-based bioreactors provides both the 2 GtC/yr of algae to satiate the grazer appetites and the remaining 1 GtC/yr of uneaten seed that remain to trigger the amplified ocean blooming of the up to 14 GtC/yr of heavier-than-water algae.
